(19) United States
(12) Patent Application Publication
(10) Pub. No.: US 2009/0215725 A1
(43) Pub. Date: Aug. 27, 2009

(54) Substituted 4-aminocyclohexane derivatives

(75) Inventors: Stefan SCHUNK, Aachen (DE); Saskia ZEMOLKA, Aachen (DE); Klaus LINZ, Wachtberg (DE); Werner ENGLBERGER, Stolberg (DE); Fitz THEIL, Berlin (DE)

Correspondence Address: NORRIS, MCLAUGHLIN & MARCUS, PA, 875 THIRD AVENUE, 18TH FLOOR, NEW YORK, NY, 10022, US

(73) Assignee: GRUNENTHAL GMBH, Aachen (DE)

(21) Appl. No.: 12/388,725

(22) Filed: Feb. 19, 2009

(30) Foreign Application Priority Data
Feb. 26, 2008 (EP) 08003442.4

(51) Int. Cl. A61K 31/695; C07F 7/10; A61K 31/4045; C07D 209/04; A61P 25/00

(52) U.S. Cl. 514/63; 548/406; 514/415; 548/469; 546/14

(57) ABSTRACT

Substituted 4-aminocyclohexane derivatives having the formula I:

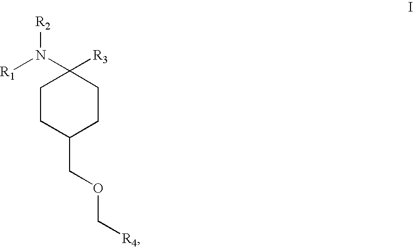

wherein R¹-R⁴ are as described herein, methods for their preparation, medicinal products and pharmaceutical compositions containing these compounds and the use of substituted 4-aminocyclohexane derivatives for treating pain.

This application claims priority of European Patent Application No. 08003442.4, filed on Feb. 26, 2008.

The present application relates to substituted 4-aminocyclohexane derivatives, methods for their preparation, medicinal products containing these compounds and the use of substituted 4-aminocyclohexane derivatives for the preparation of medicinal products.

The heptadecapeptide nociceptin is an endogenous ligand of the ORL1 (opioid receptor-like) receptor (Meunier et al., Nature 377, 1995, p. 532-535), which belongs to the family of opioid receptors, is to be found in many regions of the brain and spinal cord, and has a high affinity for the ORL1 receptor. The ORL1 receptor is homologous to the μ, κ and δ opioid receptors and the amino acid sequence of the nociceptin peptide displays a strong similarity to those of the known opioid peptides. The activation of the receptor induced by nociceptin leads via the coupling with G_{i/o}, proteins to an inhibition of the adenylate cyclase (Meunier et al., Nature 377, 1995, p. 532-535).

After intercerebroventicular application, the nociceptin peptide exhibits pronociceptive and hyperalgesic activity in various animal models (Reinscheid et al., Science 270, 1995, p. 792-794). These findings can be explained as an inhibition of stress-induced analgesia (Mogil et al., Neuroscience 75, 1996, p. 333-337). Anxiolytic activity of the nociceptin could also be demonstrated in this connection (Jenck et al., Proc. Natl. Acad. Sci. USA 94, 1997, 14854-14858).

On the other hand, an antinociceptive effect of nociceptin could also be demonstrated in various animal models, in particular after intrathaecal application. Nociceptin has an antinociceptive effect in various pain models, for example in the tail flick test in mice (King et al., Neurosci. Lett., 223, 1997, 113-116). In models of neuropathic pain, an antinociceptive effect of nociceptin could likewise be detected and was particularly beneficial since the effectiveness of nociceptin increases after axotomy of spinal nerves. This contrasts with conventional opioids, the effectiveness of which decreases under these conditions (Abdulla and Smith, J. Neurosci., 18, 1998, p. 9685-9694).

The ORL1 receptor is also involved in the regulation of further physiological and pathophysiological processes. These include inter alia learning and memory (Manabe et al., Nature, 394, 1997, p. 577-581), hearing capacity (Nishi et al., EMBO J., 16, 1997, p. 1858-1864) and numerous further processes. A synopsis by Calo et al. (Br. J. Pharmacol., 129, 2000, 1261-1283) gives an overview of the indications or biological processes in which the ORL1 receptor plays a part or could very probably play a part. Mentioned inter alia are: analgesics, stimulation and regulation of nutrient absorption, effect on μ-agonists such as morphine, treatment of withdrawal symptoms, reduction of the addiction potential of opioids, anxiolysis, modulation of motor activity, memory disorders, epilepsy; modulation of neurotransmitter release, in particular of glutamate, serotonin and dopamine, and therefore neurodegenerative diseases; influencing the cardiovascular system, triggering an erection, diuresis, anti-natriuresis, electrolyte balance, arterial blood pressure, water retention disorders, intestinal motility (diarrhoea), relaxation of the respiratory tract, micturation reflex (urinary incontinence). The use of agonists and antagonists as anorectics, analgesics (also when coadministered with opioids) or nootropics is also discussed.

The possible applications of compounds that bind to the ORL1 receptor and activate or inhibit it are correspondingly diverse. In addition, however, opioid receptors such as the μ-receptor, but also the other subtypes of these opioid receptors, namely δ and κ, play an important part in the field of pain therapy and also other of the aforementioned indications. It is accordingly beneficial if the compound also has an effect on these opioid receptors.

In the treatment of neuropathic pain with opioids, the opioids conventionally have to be administered in higher doses than would be necessary for the treatment of acute pain. The disadvantage for patients lies in the fact that they generally suffer from relatively severe characteristic opioid side-effects. It would therefore be advantageous to find compounds which allow if at all possible a lower dose, but at least no higher dose, than is necessary for the treatment of acute pain to be administered for the treatment of neuropathic pain.

WO2004043902 discloses 4-hydroxymethyl-1-arylcyclohexylamine compounds. Although indole derivatives are also mentioned inter alia, such examples are not disclosed, however, and moreover no further details are given of the way in which the indole radical is linked to the lead structure. Receptor binding data is described for four compounds, in which R⁵ denotes phenyl or 4-fluorophenyl, and effectiveness in the tail flick test, an acute pain model, is described for two compounds. In comparison to chronic or neuropathic pain, however, acute pain is often readily treated, such that there is a need in particular for new medicinal products for the treatment of chronic and neuropathic pain.

There is also a need for compounds having at most a low affinity to the hERG ion channel, to the L-type calcium ion channel (phenyl alkylamine, benzothiazepine, dihydropyridine binding sites) or to the sodium channel in the BTX assay (batrachotoxin), since otherwise this can be interpreted as an indication of cardiovascular side-effects. Furthermore, the solubility in aqueous media should be adequate, since otherwise this can have a negative effect inter alia on bioavailability. The chemical stability of the compounds should also be adequate. If the compounds do not have adequate pH, UV or oxidation stability, this can have a negative effect inter alia on storage stability and also on oral bioavailability. A favourable PK/PD (pharmacokinetic/pharmacodynamic) profile is also desirable so that, for example, the period of action is not too long. The metabolic stability, which can have an effect on an increased bioavailability, should also not be too low. A weak or non-existent interaction with transporter molecules, which are involved in the uptake and excretion of medicinal products, can also be taken as an indication of improved bioavailability and at most low medicinal product interaction. Furthermore, interactions with the enzymes involved in the breakdown and excretion of medicinal products should also be as low as possible, since such test results likewise indicate that at most low or even no medicinal product interactions whatsoever are to be anticipated.

The object underlying the invention is to provide compounds which are suitable for pharmaceutical purposes and which offer advantages over the prior art compounds. The compounds should be suitable in particular for the treatment of chronic and neuropathic pain.

This object is achieved by the subject matter described hereinbelow.

### SUMMARY OF THE INVENTION

Surprisingly it has been found that substituted 4-aminocyclohexane derivatives having the general formula I are particularly well suited to the treatment of chronic and neuropathic pain.

The invention provides substituted 4-aminocyclohexane derivatives having the general formula I,

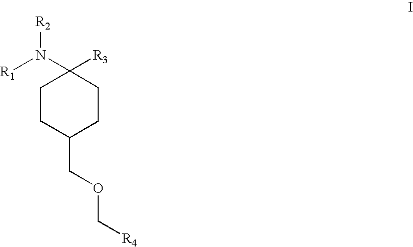

wherein
R¹ and R² mutually independently denote C₁₋₃ alkyl or H or the radicals R¹ and R² form a ring with inclusion of the N atom and together denote (CH₂)₃ or (CH₂)₄;
R³ optionally denotes aryl or heteroaryl bound by a C₁₋₃ alkyl chain, each unsubstituted or mono- or polysubstituted; or C₁₋₆ alkyl, unsubstituted or mono- or polysubstituted;
R⁴ denotes indolyl, pyrrolo[2,3-b]pyridinyl, pyrrolo[2,3-c]pyridinyl, pyrrolo[3,2-c]pyridinyl or pyrrolo[3,2-b]pyridinyl, each unsubstituted or mono- or polysubstituted;
in the form of the racemate; the enantiomers, diastereomers, mixtures of enantiomers or diastereomers or a single enantiomer or diastereomer; the bases and/or salts of physiologically compatible acids or cations.

### BRIEF DESCRIPTION OF THE DRAWING

The invention will now be described in greater detail with reference to the drawing, wherein:

FIG. 1 is a graph depicting the analysis of Example 10 hereinbelow in the Chung model.

### DETAILED DESCRIPTION OF THE INVENTION

Within the meaning of this invention the expressions “C₁₋₆ alkyl” and “C₁₋₃ alkyl” include acyclic saturated or unsaturated hydrocarbon radicals, which can be branched or straight-chain and unsubstituted or mono- or polysubstituted, having respectively 1, 2, 3, 4, 5 or 6 C atoms or 1, 2 or 3 C atoms, i.e. C₁₋₅ alkanyls, C₂₋₅ alkenyls and C₂₋₅ alkynyls or C₁₋₃ alkanyls, C₂₋₃ alkenyls and C₂₋₃ alkynyls. Alkenyls have at least one C—C double bond and alkynyls have at least one C—C triple bond. Alkyl is advantageously selected from the group comprising methyl, ethyl, n-propyl, 2-propyl, n-butyl, isobutyl, sec-butyl, tert-butyl, n-pentyl, isopentyl, neopentyl, n-hexyl, 2-hexyl; ethylenyl (vinyl), ethynyl, propenyl (—CH₂CH═CH₂, —CH═CH—CH₃, —C(═CH₂)—CH₃), propynyl (—CH—C≡CH, —C≡C—CH₃), 1,1-dimethylethyl, 1,1-dimethylpropyl, butenyl, butynyl, pentenyl, pentynyl, hexyl, hexenyl or hexynyl. Methyl and ethyl are particularly preferred within the meaning of this invention.

For the purposes of this invention the expression “cycloalkyl” denotes cyclic hydrocarbons having 3, 4, 5, 6, 7 or 8 carbon atoms, wherein the hydrocarbons can be saturated or unsaturated (but not aromatic), unsubstituted or mono- or polysubstituted. Cycloalkyl is advantageously selected from the group including cyclopropyl, cyclobutyl, cyclopentyl, cyclohexyl, cycloheptyl, cyclooctyl, cyclopentenyl, cyclohexenyl, cycloheptenyl and cyclooctenyl. Cyclobutyl, cyclopentyl and cyclohexyl are particularly preferred within the meaning of this invention.

The term (CH₂)₃₋₆ is understood to mean —CH₂—CH₂—CH₂—, —CH₂—CH₂—CH₂—CH₂—, —CH₂—CH₂—CH₂—CH₂—CH₂— and CH₂—CH₂—CH₂—CH₂—CH₂—CH₂—.

Within the meaning of this invention the expression “aryl” denotes carbocyclic ring systems having up to 14 ring members with at least one aromatic ring, but without heteroatoms in only one of the rings, inter alia phenyls, naphthyls and phenanthrenyls. The aryl radicals can also be fused to other saturated, (partially) unsaturated or aromatic ring systems. Each aryl radical can be present in unsubstituted or mono- or polysubstituted form, wherein the aryl substituents can be identical or different and can be at any desired and possible position of the aryl. Phenyl or naphthyl radicals are particularly advantageous.

The expression “heteroaryl” stands for a 5-, 6- or 7-membered cyclic aromatic radical containing at least 1, optionally also 2, 3, 4 or 5 heteroatoms, wherein the heteroatoms can be identical or different and the heterocyclic compound can be unsubstituted or mono- or polysubstituted; if the heterocyclic compound is substituted, the substituents can be identical or different and can be at any desired and possible position of the heteroaryl. The heterocyclic compound can also be part of a bicyclic or polycyclic system having up to 14 ring members. Preferred heteroatoms are nitrogen, oxygen and sulfur. It is preferable for the heteroaryl radical to be selected from the group including pyrrolyl, indolyl, furyl (furanyl), benzofuranyl, thienyl (thiophenyl), benzothienyl, benzothiadiazolyl, benzothiazolyl, benzotriazolyl, benzodioxolanyl, benzodioxanyl, phthalazinyl, pyrazolyl, imidazolyl, thiazolyl, oxazolyl, isoxazolyl, pyridinyl, pyridazinyl, pyrimidinyl, pyrazinyl, pyranyl, indazolyl, purinyl, indolizinyl, quinolinyl, isoquinolinyl, quinazolinyl, carbazolyl, phenazinyl, phenothiazinyl or oxadiazolyl, wherein the binding to the compounds having the general structure I can be made via any desired and possible ring member of the heteroaryl radical.

In connection with definitions of substituents, “alkyl” denotes “C₁₋₆ alkyl” unless otherwise specified.

In connection with “alkyl”, the term “substituted” within the meaning of this invention is understood to mean the substitution of one or more hydrogen radicals with F, Cl, Br, I, —CN, NH₂, NH-alkyl, NH-aryl, NH-heteroaryl, NH-cycloalkyl, NH-alkyl-aryl, NH-alkyl-heteroaryl, NH-alkyl-OH, N(alkyl)₂, N(alkyl-aryl)₂, N(alkyl-heteroaryl)₂, N(cycloalkyl)₂, N(alkyl-OH)₂, NO₂, SH, S-alkyl, S-aryl, S-heteroaryl, S-alkyl-aryl, S-alkyl-heteroaryl, S-cycloalkyl, S-alkyl-OH, S-alkyl-SH, OH, O-alkyl, O-aryl, O-heteroaryl, O-alkyl-aryl, O-alkyl-heteroaryl, O-cycloalkyl, O-alkyl-OH, CHO, C(═O)C₁₋₆ alkyl, C(═S)C₁₋₆ alkyl, C(═O)aryl, C(═S)aryl, C(═O)C₁₋₆ alkyl-aryl, C(═S)C₁₋₆ alkyl-aryl, C(═O)-heteroaryl, C(═S)-heteroaryl, C(═O)-cycloalkyl, C(═S)-cycloalkyl, CO₂H, CO₂ alkyl, CO₂ alkyl-aryl, C(═O)NH₂, C(═O)NH-alkyl, C(═O)NH-aryl, C(═O)NH-cycloalkyl, C(═O)N(alkyl)₂, C(═O)N(alkyl-aryl)₂, C(═O)N(alkyl-heteroaryl)₂, C(═O)N(cycloalkyl)₂, SO-alkyl, SO₂-alkyl, SO₂NH₂, SO₃H, PO(O—C₁₋₆ alkyl)₂═O, ═S, wherein polysubstituted radicals are understood to mean radicals which are either substituted multiple times, e.g. twice or three times, at different or the same atoms, for example three times at the same C atom, as in the case of CF₃ or —CH₂CF₃, or at different sites, as in the case of —CH(OH)—CH═CH—CHCl₂. The polysubstitution can take place with identical or with different substituents. A substituent can also optionally itself be substituted, so —O alkyl also includes inter alia —O—CH₂—CH₂—O—CH₂—CH₂—OH. It is preferable within the meaning of this invention for alkyl to be substituted with F, Cl, Br, I, CN, CH₃, C₂H₅, NH₂, NO₂, SH, CF₃, OH, OCH₃, cyclopentyl, cyclohexyl, OC₂H₅ or N(CH₃)₂, preferably F, Cl, Br, I, CN, CH₃, C₂H₅, NH₂, NO₂, SH, CF₃, OH, OCH₃, OC₂H₅ or N(CH₃)₂. It is most particularly preferable for alkyl to be substituted with OH, OCH₃ or OC₂H₅.

In connection with “aryl”, “indole”, “pyrrolo[2,3-b]pyridine”, “pyrrolo[2,3-c]pyridine”, “pyrrolo[3,2-c]pyridine”, “pyrrolo[3,2-b]pyridine” or “heteroaryl”, “mono- or polysubstituted” within the meaning of this invention is understood to mean the single or multiple, e.g. two, three, four or five times, substitution of one or more hydrogen atoms in the ring system with F, Cl, Br, I, CN, NH₂, NH-alkyl, NH-aryl, NH-heteroaryl, NH-alkyl-aryl, NH-alkyl-heteroaryl, NH-cycloalkyl, NH-alkyl-OH, N(alkyl)₂, N(alkyl-aryl)₂, N(alkyl-heteroaryl)₂, N(cycloalkyl)₂, N(alkyl-OH)₂, NO₂, SH, S-alkyl, S-cycloalkyl, S-aryl, S-heteroaryl, S-alkyl-aryl, S-alkyl-heteroaryl, S-cycloalkyl, S-alkyl-OH, S-alkyl-SH, OH, O-alkyl, O-cycloalkyl, O-aryl, O-heteroaryl, O-alkyl-aryl, O-alkyl-heteroaryl, O-cycloalkyl, O-alkyl-OH, CHO, C(═O)C₁₋₆ alkyl, C(═S)C₁₋₆ alkyl, C(═O)aryl, C(═S)aryl, C(═O)—C₁₋₆ alkyl-aryl, C(═S)C₁₋₆ alkyl-aryl, C(═O)-heteroaryl, C(═S)-heteroaryl, C(═O)-cycloalkyl, C(═S)-cycloalkyl, CO₂H, CO₂-alkyl, CO₂-alkyl-aryl, C(═O)NH₂, C(═O)NH-alkyl, C(═O)NH-aryl, C(═O)NH-cycloalkyl, C(═O)N(alkyl)₂, C(═O)N(alkyl-aryl)₂, C(═O)N(alkyl-heteroaryl)₂, C(═O)N(cycloalkyl)₂, S(O)-alkyl, S(O)-aryl, SO₂-alkyl, SO₂-aryl, SO₂NH₂, SO₃H, Si(alkyl)₃, CF₃; alkyl, cycloalkyl, aryl and/or heteroaryl; at one or optionally different atoms (wherein a substituent can optionally itself be substituted). The polysubstitution is performed with identical or with different substituents. If “aryl”, “indole”, “pyrrolo[2,3-b]pyridine”, “pyrrolo[2,3-c]pyridine”, “pyrrolo[3,2-c]pyridine”, “pyrrolo[3,2-b]pyridine” or “heteroaryl” is itself substituted with an aryl or heteroaryl radical optionally bound via a bridge, this substituent is preferably itself unsubstituted or mono- or polysubstituted with F, Cl, Br, I, CN, CH₃, C₂H₅, NH₂, NO₂, SH, CF₃, OH, OCH₃, OC₂H₅ or N(CH₃)₂.

It is particularly preferred within the meaning of this invention for “aryl”, “indole”, “pyrrolo[2,3-b]pyridine”, “pyrrolo[2,3-c]pyridine”, “pyrrolo[3,2-c]pyridine”, “pyrrolo[3,2-b]pyridine” or “heteroaryl” to be substituted with F, Cl, Br, I, CN, CH₃, C₂H₅, NH₂, tert-butyl, Si(ethyl)₃, Si(methyl)₂(tert-butyl), SO₂CH₃, C(O)CH₃, NO₂, SH, CF₃, OH, OCH₃, OC₂H₅ or N(CH₃)₂.

The term salt is understood to mean any form of the active ingredient according to the invention in which it assumes an ionic form or is charged and is coupled to a counterion (a cation or anion) or is in solution. Also included here are complexes of the active ingredient with other molecules and ions, in particular complexes which are complexed by means of ionic interactions. It means in particular (and this is also a preferred embodiment of this invention) physiologically compatible salts, in particular physiologically compatible salts with cations or bases and physiologically compatible salts with anions or acids or also a salt formed with a physiologically compatible acid or a physiologically compatible cation.

Within the meaning of this invention the term “physiologically compatible salt with anions or acids” is understood to mean salts of at least one of the compounds according to the invention—mostly protonated, for example on nitrogen—as cation with at least one anion, which are physiologically—particularly when used in humans and/or mammals—compatible. Within the meaning of this invention this is particularly understood to mean the salt formed with a physiologically compatible acid, namely salts of the individual active ingredient with inorganic or organic acids which are physiologically—particularly when used in humans and/or mammals—compatible. Examples of physiologically compatible salts of certain acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, saccharinic acid, monomethyl sebacic acid, 5-oxoproline, hexane-1-sulfonic acid, nicotinic acid, 2-, 3- or 4-aminobenzoic acid, 2,4,6-trimethylbenzoic acid, α-lipoic acid, acetylglycine, acetyl salicylic acid, hippuric acid and/or aspartic acid. The hydrochloride salt, the citrate and the hemicitrate are particularly preferred.

Within the meaning of this invention the term “salt formed with a physiologically compatible acid” is understood to mean salts of the individual active ingredient with inorganic or organic acids which are physiologically—particularly when used in humans and/or mammals—compatible. The hydrochloride and the citrate are particularly preferred. Examples of physiologically compatible acids are: hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid, saccharinic acid, monomethyl sebacic acid, 5-oxoproline, hexane-1-sulfonic acid, nicotinic acid, 2-, 3- or 4-aminobenzoic acid, 2,4,6-trimethylbenzoic acid, α-lipoic acid, acetylglycine, acetyl salicylic acid, hippuric acid and/or aspartic acid.

Within the meaning of this invention the term “physiologically compatible salt with cations or bases” is understood to mean salts of at least one of the compounds according to the invention—mostly a (deprotonated) acid—as anion with at least one, preferably inorganic, cation, which are physiologically—particularly when used in humans and/or mammals—compatible. Particularly preferred are the salts of the alkali and alkaline-earth metals, but also ammonium salts, but in particular (mono) or (di)sodium, (mono) or (di)potassium, magnesium or calcium salts.

Within the meaning of this invention the term “salt formed with a physiologically compatible cation” is understood to mean salts of at least one of the compounds as anion with at least one inorganic cation, which is physiologically—particularly when used in humans and/or mammals—compatible. Particularly preferred are the salts of the alkali and alkaline-earth metals, but also ammonium salts, but in particular (mono) or (di)sodium, (mono) or (di)potassium, magnesium or calcium salts.

Preferred within the meaning of this invention are substituted 4-aminocyclohexane derivatives wherein

“alkyl substituted” stands for the substitution of a hydrogen radical with F, Cl, Br, I, —CN, NH₂, NH—C₁₋₆ alkyl, NH—C₁₋₆ alkyl-OH, C₁₋₆ alkyl, N(C₁₋₆ alkyl)₂, N(C₁₋₆ alkyl-OH)₂, NO₂, SH, S—C₁₋₆ alkyl, S-benzyl, O—C₁₋₆ alkyl, OH, O—C₁₋₆ alkyl-OH, ═O, O-benzyl, C(═O)C₁₋₆ alkyl, C(═O)OC₁₋₆ alkyl, phenyl or benzyl,
and “aryl-substituted”, “indole-substituted”, “pyrrolo[2,3-b]pyridine-substituted”, “pyrrolo[2,3-c]pyridine-substituted”, “pyrrolo[3,2-c]pyridine-substituted”, “pyrrolo[3,2-b]pyridine-substituted” or “heteroaryl-substituted” stands for the single or multiple, e.g. two, three or four times, substitution of one or more hydrogen atoms in the ring system with F, Cl, Br, I, CN, NH₂, NH—C₁₋₆ alkyl, NH—C₁₋₆ alkyl-OH, N(C₁₋₆ alkyl)₂, N(C₁₋₆ alkyl-OH)₂, NO₂, SH, S—C₁₋₆ alkyl, OH, O—C₁₋₆ alkyl, O—C₁₋₆ alkyl-OH, C(═O)-aryl; C(═O)C₁₋₆ alkyl, C(═O)NHC₁₋₆ alkyl; C(═O)—N-morpholine; C(═O)-piperidine; (C═O)-pyrrolidine; (C═O)-piperazine; NHSO₂C₁₋₆ alkyl, NHCOC₁₋₆ alkyl, CO₂H, CH₂SO₂ phenyl, SO₂ alkyl, Si(alkyl)₃, CO₂—C₁₋₆ alkyl, OCF₃, CF₃,

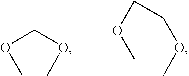

C₁₋₆ alkyl, benzyloxy, phenoxy, phenyl, pyridyl, alkylaryl, thienyl or furyl, wherein aryl and heteroaryl substituents can themselves be substituted with F, Cl, Br, I, CN, CH₃, C₂H₅, NH₂, NO₂, SH, CF₃, OH, OCH₃, OC₂H₅ or N(CH₃)₂,
in the form of the racemate; the enantiomers, diastereomers, mixtures of enantiomers or diastereomers or a single enantiomer or diastereomer; the bases and/or salts of physiologically compatible acids or cations.

For a preferred embodiment of the substituted 4-aminocyclohexane derivatives according to the invention,

R¹ and R² mutually independently denote methyl or H or the radicals R¹ and R² form a ring with inclusion of the N atom and denote (CH₂)₃ or (CH₂)₄.

Substituted 4-aminocyclohexane derivatives are most particularly preferred wherein R¹ and R² mutually independently denote methyl or H, preferably methyl.

Also preferred are substituted 4-aminocyclohexane derivatives wherein R³ stands for phenyl, benzyl or phenethyl, each unsubstituted or mono- or polysubstituted at the ring; C₁₋₆ alkyl, unsubstituted or mono- or polysubstituted; pyridyl, thienyl, thiazolyl, imidazolyl, 1,2,4-triazolyl or benzimidazolyl, unsubstituted or mono- or polysubstituted.

Particularly preferred are substituted indole derivatives having the general formula I, wherein R³ stands for phenyl, benzyl, phenethyl, thienyl, pyridyl, thiazolyl, imidazolyl, 1,2,4-triazolyl, benzimidazolyl or benzyl, unsubstituted or mono- or polysubstituted with F, Cl, Br, CN, CH₃, C₂H₅, NH₂, NO₂, SH, CF₃, OH, OCH₃, OC₂H₅ or N(CH₃)₂; ethyl, n-propyl, 2-propyl, allyl, n-butyl, isobutyl, sec-butyl, tert-butyl, n-pentyl, isopentyl, neopentyl, n-hexyl, cyclopentyl or cyclohexyl, each unsubstituted or mono- or polysubstituted with OH, OCH₃ or OC₂H₅,

wherein thienyl, pyridyl, thiazolyl, imidazolyl, 1,2,4-triazolyl and benzimidazolyl are preferably unsubstituted;
in particular
phenyl, unsubstituted or monosubstituted with F, Cl, CN, CH₃; thienyl; or n-butyl, unsubstituted or mono- or polysubstituted with OCH₃, OH or OC₂H₅, in particular with OCH₃.

It is preferable for R⁴ to denote indole, pyrrolo[2,3-b]pyridine, pyrrolo[2,3-c]pyridine, pyrrolo[3,2-c]pyridine or pyrrolo[3,2-b]pyridine, each unsubstituted or mono- or polysubstituted with F, Cl, Br, I, CN, CH₃, C₂H₅, NH₂, tert-butyl, Si(ethyl)₃, Si(methyl)₂(tert-butyl), SO₂CH₃, C(O)CH₃, NO₂, SH, CF₃, OH, OCH₃, OC₂H₅ or N(CH₃)₂, in particular Si(ethyl)₃, Si(methyl)₂(tert-butyl), CN, CF₃, F, C(O)CH₃, SO₂CH₃ or CH₃.

Also preferred are substituted 4-aminocyclohexane derivatives wherein R⁴ stands for

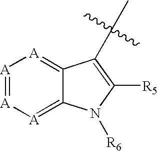

wherein
A stands for N or CR⁷⁻¹⁰, wherein A denotes at most one N;
R⁵, R⁷, R⁸, R⁹ and R¹⁰ mutually independently stand for H, F, Cl, Br, CN, CH₃, C₂H₅, NH₂, tert-butyl, Si(ethyl)₃, Si(methyl)₂(tert-butyl), SO₂CH₃, C(O)CH₃, NO₂, SH, CF₃, OH, OCH₃, OC₂H₅ or N(CH₃)₂, in particular Si(ethyl)₃, Si(methyl)₂(tert-butyl), CN, CF₃, F, C(O)CH₃, SO₂CH₃ or CH₃, and
R⁶ stands for H, CH₃ or C(O)CH₃,
in particular
R⁴ denotes one of the structures L, M or N

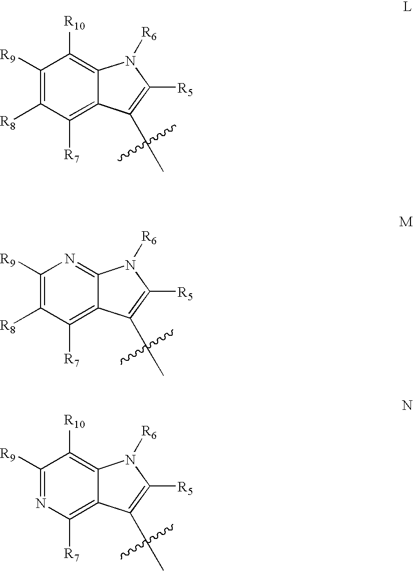

wherein the radicals R⁵, R⁷, R⁸, R⁹ and R¹⁰ mutually independently stand for H, F, Cl, Br, CN, CH₃, C₂H₅, NH₂, tert-butyl, Si(ethyl)₃, Si(methyl)₂(tert-butyl), SO₂CH₃, C(O)CH₃, NO₂, SH, CF₃, OH, OCH₃, OC₂H₅ or N(CH₃)₂, in particular Si(ethyl)₃, Si(methyl)₂(tert-butyl), CN, CF₃, F, C(O)CH₃, SO₂CH₃ or CH₃, and
R⁶ stands for H, CH₃ or C(O)CH₃,
it being preferable for R⁴ to stand for the substructure having the general formula L.

In the 4-aminocyclohexane derivatives according to the invention R⁴ is preferably bound by one of its C atoms to the general formula I.

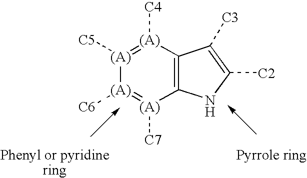

In a preferred embodiment R⁴ is bound to the general formula I by its C2 or C3 atom in the pyrrole ring. In another preferred embodiment R⁴ is bound to the general formula I by its C4, C5, C6 or C7 atom, if present, in the phenyl or pyridine ring.

Also preferred are substituted 4-aminocyclohexane derivatives wherein R⁴ stands for

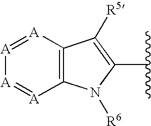

wherein
A stands for N or CR⁷⁻¹⁰, wherein A denotes at most one N;
R^{5′}, R⁷, R⁸, R⁹ and R¹⁰ mutually independently stand for H, F, Cl, Br, CN, CH₃, C₂H₅, NH₂, tert-butyl, Si(ethyl)₃, Si(methyl)₂(tert-butyl), SO₂CH₃, SO₂C₆H₅, SO₂C₆H₄—CH₃, C(O)CH₃, NO₂, SH, CF₃, OCF₃, OH, OCH₃, OC₂H₅ or N(CH₃)₂, and
R⁶ stands for H, CH₃ or C(O)CH₃;
in particular for one of the structures O₂, P₂, Q₂, R₂ or S₂

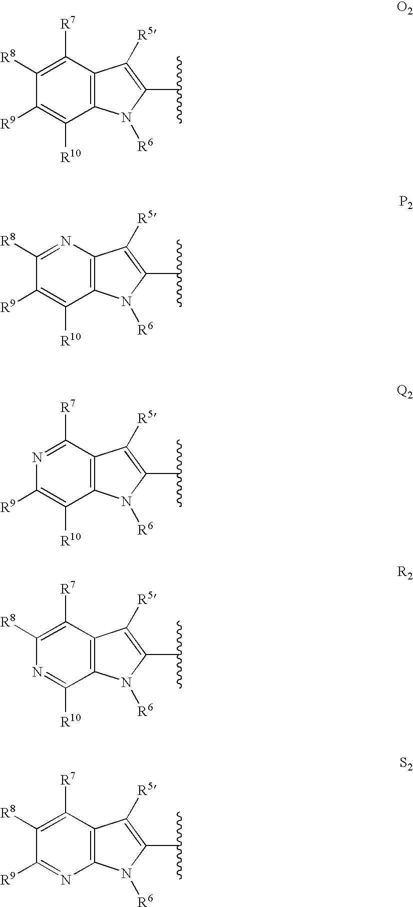

Also preferred are substituted 4-aminocyclohexane derivatives wherein R⁴ stands for

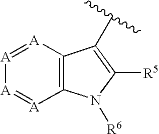

wherein
A stands for N or CR⁷⁻¹⁰, wherein A denotes at most one N;
R⁵, R⁷, R⁸, R⁹ and R¹⁰ mutually independently stand for H, F, Cl, Br, CN, CH₃, C₂H₅, NH₂, tert-butyl, Si(ethyl)₃, Si(methyl)₂(tert-butyl), SO₂CH₃, SO₂C₆H₅, SO₂C₆H₄—CH₃, C(O)CH₃, NO₂, SH, CF₃, OCF₃, OH, OCH₃, OC₂H₅ or N(CH₃)₂, and
R⁶ stands for H, CH₃ or C(O)CH₃;
in particular for one of the structures O₃, P₃, Q₃, R₃ or S₃

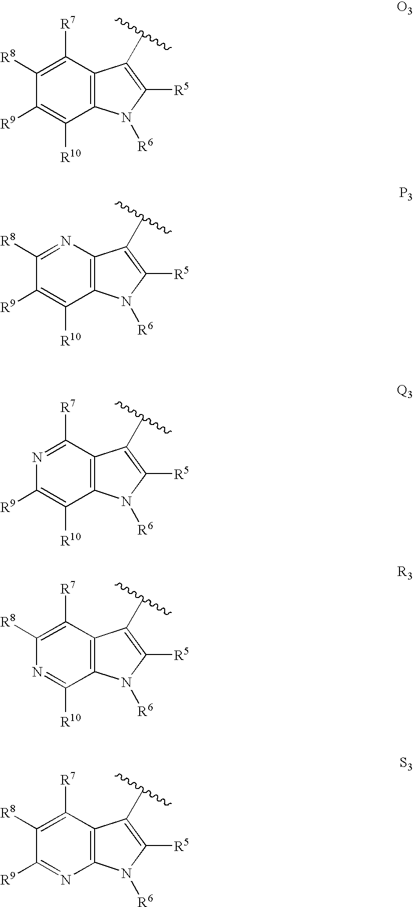

Also preferred are substituted 4-aminocyclohexane derivatives wherein R⁴ stands for

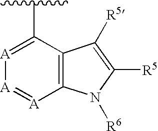

wherein
A stands for N or CR⁸⁻¹⁰, wherein A denotes at most one N;
R⁵, R^{5′}, R⁸, R⁹ and R¹⁰ mutually independently stand for H, F, Cl, Br, CN, CH₃, C₂H₅, NH₂, tert-butyl, Si(ethyl)₃, Si(methyl)₂(tert-butyl), SO₂CH₃, SO₂C₆H₅, SO₂C₆H₄—CH₃, C(O)CH₃, NO₂, SH, CF₃, OCF₃, OH, OCH₃, OC₂H₅ or N(CH₃)₂, and
R⁶ stands for H, CH₃ or C(O)CH₃;
in particular for one of the structures O₄, Q₄, R₄ or S₄

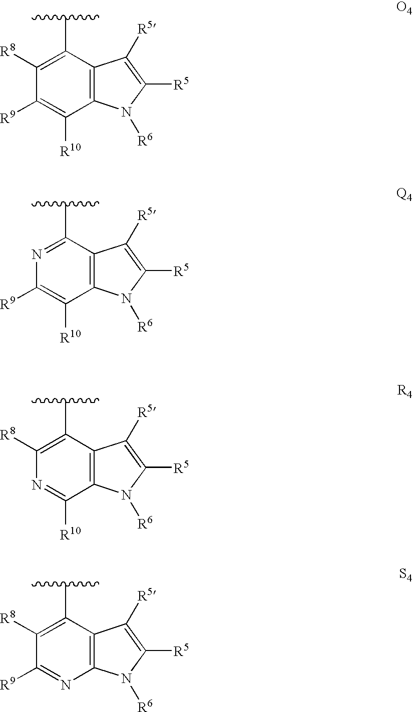

Also preferred are substituted 4-aminocyclohexane derivatives wherein R⁴ stands for

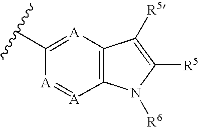

wherein
A stands for N or CR^{7,9,10}, wherein A denotes at most one N;
R⁵, R^{5′}, R⁷, R⁹ and R¹⁰ mutually independently stand for H, F, Cl, Br, CN, CH₃, C₂H₅, NH₂, tert-butyl, Si(ethyl)₃, Si(methyl)₂(tert-butyl), SO₂CH₃, SO₂C₆H₅, SO₂C₆H₄—CH₃, C(O)CH₃, NO₂, SH, CF₃, OCF₃, OH, OCH₃, OC₂H₅ or N(CH₃)₂, and
R⁶ stands for H, CH₃ or C(O)CH₃;
in particular for one of the structures O₅, P₅, R₅ or S₅

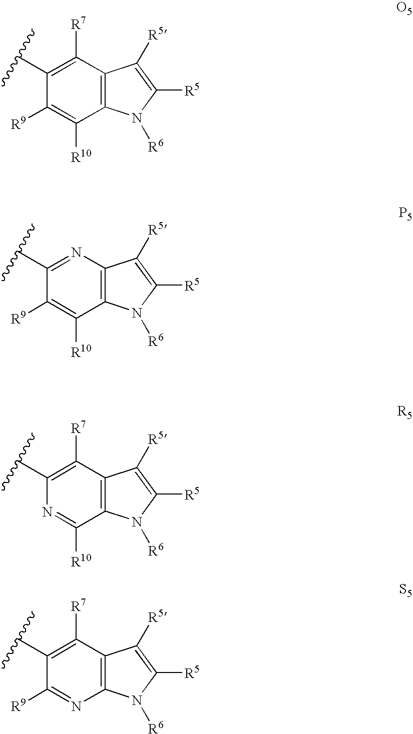

Also preferred are substituted 4-aminocyclohexane derivatives wherein R⁴ stands for

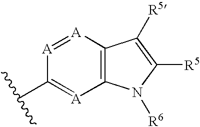

wherein
A stands for N or CR^{7,8,10}, wherein A denotes at most one N;
R⁵, R^{5′}, R⁷, R⁸ and R¹⁰ mutually independently stand for H, F, Cl, Br, CN, CH₃, C₂H₅, NH₂, tert-butyl, Si(ethyl)₃, Si(methyl)₂(tert-butyl), SO₂CH₃, SO₂C₆H₅, SO₂C₆H₄—CH₃, C(O)CH₃, NO₂, SH, CF₃, OCF₃, OH, OCH₃, OC₂H₅ or N(CH₃)₂, and
R⁶ stands for H, CH₃ or C(O)CH₃;
in particular for one of the structures O₆, P₆, Q₆ or S₆

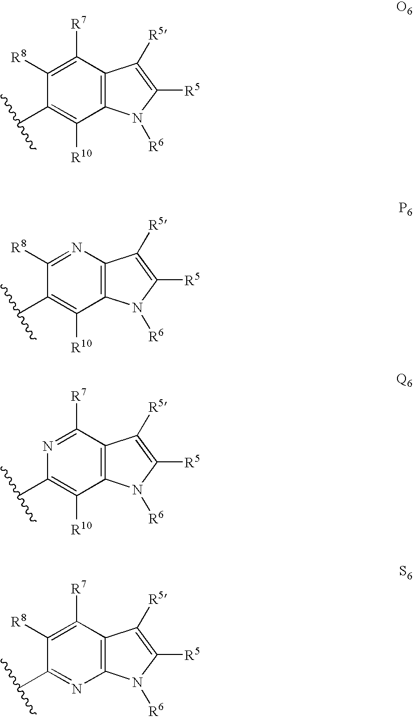

Also preferred are substituted 4-aminocyclohexane derivatives wherein R⁴ stands for

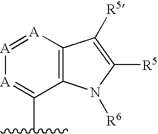

wherein
A stands for N or CR⁷⁻⁹, wherein A denotes at most one N;
R⁵, R^{5′}, R⁷, R⁸ and R⁹ mutually independently stand for H, F, Cl, Br, CN, CH₃, C₂H₅, NH₂, tert-butyl, Si(ethyl)₃, Si(methyl)₂(tert-butyl), SO₂CH₃, SO₂C₆H₅, SO₂C₆H₄—CH₃, C(O)CH₃, NO₂, SH, CF₃, OCF₃, OH, OCH₃, OC₂H₅ or N(CH₃)₂, and
R⁶ stands for H, CH₃ or C(O)CH₃;
in particular for one of the structures O₇, P₇, Q₇ or R₇

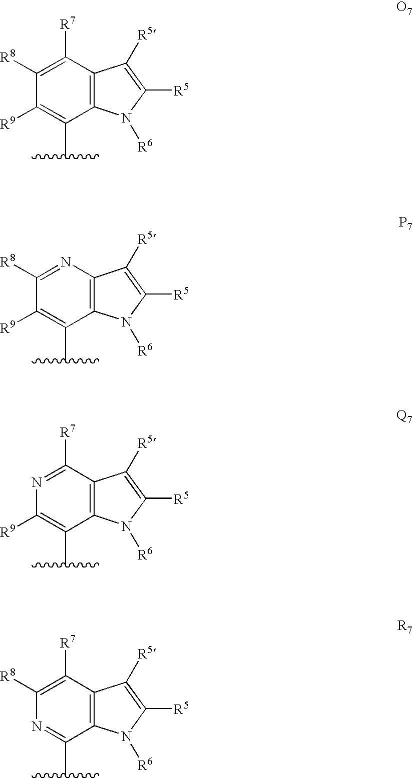

Most particularly preferred are substituted 4-aminocyclohexane derivatives from the group comprising:
1 [1-Phenyl-4-(2-triethylsilanyl-1H-indol-3-ylmethoxymethyl)cyclohexyl]dimethylamine (optionally one of two possible diastereomers)
2 [1-Phenyl-4-(1H-indol-3-ylmethoxymethyl)cyclohexyl]dimethylamine (optionally one of two possible diastereomers)
3 1-Phenyl-4-(((2-(tert-butyldimethylsilyl)-5-cyano-1H-indol-3-yl)methoxy)methyl)-N,N-dimethylcyclohexanamine (optionally one of two possible diastereomers)
4 1-Phenyl-4-(((5-cyano-2-(triethylsilyl)-1H-indol-3-yl)methoxy)methyl)-N,N-dimethylcyclohexanamine (optionally one of two possible diastereomers)
5 1-Phenyl-4-(((5-cyano-1H-indol-3-yl)methoxy)methyl)-N,N-dimethylcyclohexanamine (optionally one of two possible diastereomers)
6 1-Phenyl-4-(((2-(tert-butyldimethylsilyl)-5-trifluoromethyl-1H-indol-3-yl)methoxy)methyl)-N,N-dimethylcyclohexanamine (optionally one of two possible diastereomers)
7 1-Phenyl-4-(((5-trifluoromethyl-2-(triethylsilyl)-1H-indol-3-yl)methoxy)methyl)-N,N-dimethylcyclohexanamine (optionally one of two possible diastereomers)
8 1-Phenyl-4-(((5-trifluoromethyl-1H-indol-3-yl)methoxy)methyl)-N,N-dimethylcyclohexanamine (optionally one of two possible diastereomers)
9 1-Phenyl-4-(((5-fluoro-2-(triethylsilyl)-1H-indol-3-yl)methoxy)methyl)-N,N-dimethylcyclohexanamine (optionally one of two possible diastereomers)
10 1-Phenyl-4-(((5-fluoro-1H-indol-3-yl)methoxy)methyl)-N,N-dimethylcyclohexanamine (optionally one of two possible diastereomers)
11 1-Phenyl-N,N-dimethyl-4-(((2-(triethylsilyl)-1H-pyrrolo[2,3-b]pyridin-3-yl)methoxy)methyl)cyclohexanamine (optionally one of two possible diastereomers)
12 4-(((1H-Pyrrolo[2,3-b]pyridin-3-yl)methoxy)methyl)-1-phenyl-N,N-dimethylcyclohexanamine (optionally one of two possible diastereomers)
13 1-Phenyl-4-(((2-(tert-butyldimethylsilyl)-1H-pyrrolo[3,2-c]pyridin-3-yl)methoxy)methyl)-N,N-dimethylcyclohexanamine (optionally one of two possible diastereomers)
14 1-Phenyl-N,N-dimethyl-4-(((2-(triethylsilyl)-1H-pyrrolo[3,2-c]pyridin-3-yl)methoxy)methyl)cyclohexanamine (optionally one of two possible diastereomers)
15 4-(((1H-Pyrrolo[3,2-c]pyridin-3-yl)methoxy)methyl)-1-phenyl-N,N-dimethylcyclohexanamine (optionally one of two possible diastereomers)
16 1-(3-(((4-(Dimethylamino)-4-phenylcyclohexyl)methoxy)methyl)-5-fluoro-2-(triethylsilyl)-1H-indol-1-yl)ethanone (optionally one of two possible diastereomers)
17 4-(((5-Fluoro-1-(methylsulfonyl)-2-(triethylsilyl)-1H-indol-3-yl)methoxy)methyl)-N,N-dimethyl-1-phenylcyclohexanamine (optionally one of two possible diastereomers)
18 1-Phenyl-4-[2-(tert-butyldimethylsilanyl)-1H-indol-3-ylmethoxymethyl]cyclohexyldimethylamine (optionally one of two possible diastereomers)
19 1-Phenyl-4-(((2-(tert-butyldimethylsilyl)-5-fluoro-1H-indol-3-yl)methoxy)methyl)-N,N-dimethylcyclohexanamine (optionally one of two possible diastereomers)
20 1-Phenyl-4-(((2-(tert-butyldimethylsilyl)-1H-pyrrolo[2,3-b]pyridin-3-yl)methoxy)methyl)-N,N-dimethylcyclohexanamine (optionally one of two possible diastereomers)
21 4-(((5-Fluoro-3-methyl-1H-indol-2-yl)methoxy)methyl)-N,N-dimethyl-1-phenylcyclohexanamine (optionally one of two possible diastereomers)
22 {1-Benzyl-4-[2-(tert-butyldimethylsilanyl)-1H-indol-3-ylmethoxymethyl]cyclohexyl}dimethylamine (optionally non-polar diastereomer)
23 [1-Benzyl-4-(2-triethylsilanyl-1H-indol-3-ylmethoxymethyl)cyclohexyl] dimethylamine (optionally non-polar diastereomer)
24 [1-Benzyl-4-(1H-indol-3-ylmethoxymethyl)cyclohexyl]dimethylamine (optionally non-polar diastereomer)
25 1-Benzyl-4-(((5-fluoro-2-(triethylsilyl)-1H-indol-3-yl)methoxy)methyl)-N,N-dimethylcyclohexanamine (optionally non-polar diastereomer)
26 1-Benzyl-4-(((2-(tert-butyldimethylsilyl)-5-fluoro-1H-indol-3-yl)methoxy)methyl)-N,N-dimethylcyclohexanamine (optionally non-polar diastereomer)
27 1-Benzyl-4-(((5-fluoro-1H-indol-3-yl)methoxy)methyl)-N,N-dimethylcyclohexanamine (optionally non-polar diastereomer)
28 1-Benzyl-N,N-dimethyl-4-(((2-(triethylsilyl)-1H-pyrrolo[2,3-b]pyridin-3-yl)methoxy)methyl)cyclohexanamine (optionally non-polar diastereomer)
29 1-Benzyl-4-(((2-(tert-butyldimethylsilyl)-1H-pyrrolo[2,3-b]pyridin-3-yl)methoxy)methyl)-N,N-dimethylcyclohexanamine (optionally non-polar diastereomer)
30 4-(((1H-Pyrrolo[2,3-b]pyridin-3-yl)methoxy)methyl)-1-benzyl-N,N-dimethylcyclohexanamine (optionally non-polar diastereomer)
31 1-Benzyl-N,N-dimethyl-4-(((2-(triethylsilyl)-1H-pyrrolo[3,2-c]pyridin-3-yl)methoxy)methyl)cyclohexanamine (optionally non-polar diastereomer)
32 1-Benzyl-4-(((2-(tert-butyldimethylsilyl)-1H-pyrrolo[3,2-c]pyridin-3-yl)methoxy)methyl)-N,N-dimethylcyclohexanamine (optionally non-polar diastereomer)
33 4-(((1H-Pyrrolo[3,2-c]pyridin-3-yl)methoxy)methyl)-1-benzyl-N,N-dimethylcyclohexanamine (optionally non-polar diastereomer)
34 1-Benzyl-N,N-dimethyl-4-(((2-(triethylsilyl)-1H-indol-3-yl)methoxy)methyl)cyclohexanamine (optionally polar diastereomer)
35 1-Benzyl-4-(((2-(tert-butyldimethylsilyl)-1H-indol-3-yl)methoxy)methyl)-N,N-dimethylcyclohexanamine (optionally polar diastereomer)
36 4-(((1H-Indol-3-yl)methoxy)methyl)-1-benzyl-N,N-dimethylcyclohexanamine (optionally polar diastereomer)
37 1-Benzyl-4-(((5-fluoro-2-(triethylsilyl)-1H-indol-3-yl)methoxy)methyl)-N,N-dimethylcyclohexanamine (optionally polar diastereomer)
38 1-Benzyl-4-(((2-(tert-butyldimethylsilyl)-5-fluoro-1H-indol-3-yl)methoxy)methyl)-N,N-dimethylcyclohexanamine (optionally polar diastereomer)
39 1-Benzyl-4-(((5-fluoro-1H-indol-3-yl)methoxy)methyl)-N,N-dimethylcyclohexanamine (optionally polar diastereomer)
40 1-Benzyl-N,N-dimethyl-4-(((2-(triethylsilyl)-1H-pyrrolo[2,3-b]pyridin-3-yl)methoxy)methyl)cyclohexanamine hydrochloride (optionally polar diastereomer)
41 4-(((1H-Pyrrolo[2,3-b]pyridin-3-yl)methoxy)methyl)-1-benzyl-N,N-dimethylcyclohexanamine (optionally polar diastereomer)
42 1-Benzyl-4-(((2-(tert-butyldimethylsilyl)-1H-pyrrolo[3,2-c]pyridin-3-yl)methoxy)methyl)-N,N-dimethylcyclohexanamine (optionally polar diastereomer)
43 1-Butyl-N,N-dimethyl-4-(((2-(triethylsilyl)-1H-indol-3-yl)methoxy)methyl)cyclohexanamine (optionally non-polar diastereomer)
44 4-(((1H-Indol-3-yl)methoxy)methyl)-1-butyl-N,N-dimethylcyclohexanamine (optionally non-polar diastereomer)
45 1-Butyl-4-(((5-fluoro-2-(triethylsilyl)-1H-indol-3-yl)methoxy)methyl)-N,N-dimethylcyclohexanamine (optionally non-polar diastereomer)
46 1-Butyl-4-(((5-fluoro-1H-indol-3-yl)methoxy)methyl)-N,N-dimethylcyclohexanamine (optionally non-polar diastereomer)
47 1-Butyl-N,N-dimethyl-4-(((2-(triethylsilyl)-1H-pyrrolo[2,3-b]pyridin-3-yl)methoxy)methyl)cyclohexanamine (optionally non-polar diastereomer)
48 4-(((1H-Pyrrolo[2,3-b]pyridin-3-yl)methoxy)methyl)-1-butyl-N,N-dimethylcyclohexanamine (optionally non-polar diastereomer)
49 1-Butyl-N,N-dimethyl-4-(((2-(triethylsilyl)-1H-pyrrolo[3,2-c]pyridin-3-yl)methoxy)methyl)cyclohexanamine (optionally non-polar diastereomer)
50 4-(((1H-Pyrrolo[3,2-c]pyridin-3-yl)methoxy)methyl)-1-butyl-N,N-dimethylcyclohexanamine (optionally non-polar diastereomer)
51 1-Butyl-4-(((5-fluoro-2-(triethylsilyl)-1H-indol-3-yl)methoxy)methyl)-N-methylcyclohexanamine (optionally non-polar diastereomer)
52 1-Butyl-N-methyl-4-(((2-(triethylsilyl)-1H-indol-3-yl)methoxy)methyl)cyclohexanamine (optionally non-polar diastereomer)
53 1-Butyl-N-methyl-4-(((2-(triethylsilyl)-5-(trifluoromethyl)-1H-indol-3-yl)methoxy)methyl)cyclohexanamine (optionally non-polar diastereomer)
54 1-Butyl-N-methyl-4-(((5-(trifluoromethyl)-1H-indol-3-yl)methoxy)methyl)cyclohexanamine (optionally non-polar diastereomer)
55 4-(((5-Fluoro-2-(triethylsilyl)-1H-indol-3-yl)methoxy)methyl)-N,N-dimethyl-1-(thiophen-2-yl)cyclohexanamine (optionally one of two possible diastereomers)
56 4-(((5-Fluoro-1H-indol-3-yl)methoxy)methyl)-N,N-dimethyl-1-(thiophen-2-yl)cyclohexanamine (optionally one of two possible diastereomers)
57 3-(((4-(Azetidin-1-yl)-4-phenylcyclohexyl)methoxy)methyl)-5-fluoro-2-(triethylsilyl)-1H-indole (optionally one of two possible diastereomers)
58 3-(((4-(Azetidin-1-yl)-4-phenylcyclohexyl)methoxy)methyl)-5-fluoro-1H-indole (optionally one of two possible diastereomers)
60 4-(((5-Fluoro-1-methyl-2-(triethylsilyl)-1H-indol-3-yl)methoxy)methyl)-N,N-dimethyl-1-phenylcyclohexanamine (optionally one of two possible diastereomers)
61 4-(((5-Fluoro-1-methyl-1H-indol-3-yl)methoxy)methyl)-N,N-dimethyl-1-phenylcyclohexanamine (optionally one of two possible diastereomers)
62 4-(((5-Fluoro-1-(methylsulfonyl)-1H-indol-3-yl)methoxy)methyl)-N,N-dimethyl-1-phenylcyclohexanamine (optionally one of two possible diastereomers)
63 1-(3-(((4-(Dimethylamino)-4-phenylcyclohexyl)methoxy)methyl)-5-fluoro-1H-indol-1-yl)ethanone (optionally one of two possible diastereomers)
64 4-(((2-tert-Butyl-5-fluoro-1H-indol-3-yl)methoxy)methyl)-N,N-dimethyl-1-phenylcyclohexanamine (optionally one of two possible diastereomers)
65 3-(((4-(Azetidin-1-yl)-4-phenylcyclohexyl)methoxy)methyl)-2-(triethylsilyl)-1H-pyrrolo[2,3-b]pyridine (optionally one of two possible diastereomers)
66 3-(((4-(Azetidin-1-yl)-4-phenylcyclohexyl)methoxy)methyl)-1H-pyrrolo[2,3-b]pyridine (optionally one of two possible diastereomers)
67 4-(((2-tert-Butyl-1H-pyrrolo[2,3-b]pyridin-3-yl)methoxy)methyl)-N,N-dimethyl-1-phenylcyclohexanamine (optionally one of two possible diastereomers)
68 N,N-Dimethyl-1-phenyl-4-(((1-(phenylsulfonyl)-1H-indol-5-yl)methoxy)methyl)cyclohexanamine (optionally one of two possible diastereomers)
69 4-(((1H-Indol-5-yl)methoxy)methyl)-N,N-dimethyl-1-phenylcyclohexanamine (optionally one of two possible diastereomers)
70 N,N-Dimethyl-1-phenyl-4-(((1-(phenylsulfonyl)-1H-indol-4-yl)methoxy)methyl)cyclohexanamine (optionally one of two possible diastereomers)
71 4-(((1H-Indol-4-yl)methoxy)methyl)-N,N-dimethyl-1-phenylcyclohexanamine (optionally one of two possible diastereomers)
72 N,N-Dimethyl-1-phenyl-4-(((2-(triethylsilyl)-5-(trifluoromethoxy)-1H-indol-3-yl)methoxy)methyl)cyclohexanamine (optionally one of two possible diastereomers)
73 N,N-Dimethyl-1-phenyl-4-(((5-(trifluoromethoxy)-1H-indol-3-yl)methoxy)methyl)cyclohexanamine (optionally one of two possible diastereomers)
   in the form of the racemate; the enantiomers, diastereomers, mixtures of enantiomers or diastereomers or a single enantiomer or diastereomer; the bases and/or salts of physiologically compatible acids or cations.

The substances according to the invention are particularly suitable for the treatment of neuropathic pain. The invention therefore also provides medicinal products containing at least one substituted 4-aminocyclohexane derivative according to the invention, optionally along with suitable additives and/or auxiliary substances and/or optionally further active ingredients.

The medicinal products according to the invention optionally contain, in addition to at least one substituted 4-aminocyclohexane derivative according to the invention, suitable additives and/or auxiliary substances, including carrier materials, fillers, solvents, diluents, dyes and/or binders, and can be administered as liquid dosage forms in the form of injection solutions, drops or juices, as semi-solid dosage forms in the form of granules, tablets, pellets, patches, capsules, plasters/spray plasters or aerosols. The choice of auxiliary substances, etc., and the amount thereof to use depend on whether the medicinal product is to be administered by oral, peroral, parenteral, intravenous, intraperitoneal, intradermal, intramuscular, intranasal, buccal, rectal or local means, for example on the skin, mucous membranes or in the eyes. Preparations in the form of tablets, pastilles, capsules, granules, drops, juices and syrups are suitable for oral administration; solutions, suspensions, easily reconstitutable dry preparations and sprays are suitable for parenteral, topical and inhalative administration. Substituted 4-aminocyclohexane derivatives according to the invention in a depot formulation, in dissolved form or in a plaster, optionally with addition of agents promoting skin penetration, are suitable preparations for percutaneous administration. Preparation forms suitable for oral or percutaneous administration can deliver the substituted 4-aminocyclohexane derivatives according to the invention on a delayed release basis. The substituted 4-aminocyclohexane derivatives according to the invention can also be used in parenteral long-term depot forms, such as implants or implanted pumps, for example. Other additional active ingredients known to the person skilled in the art can be added in principle to the medicinal products according to the invention.

The amount of active ingredient to be administered to the patient varies according to the weight of the patient, the type of administration, the indication and the severity of the illness. 0.00005 to 50 mg/kg, preferably 0.001 to 0.5 mg/kg, of at least one substituted 4-aminocyclohexane derivative according to the invention are conventionally administered.

A preferred form of the medicinal product contains a substituted 4-aminocyclohexane derivative according to the invention as a pure diastereomer and/or enantiomer, as a racemate or as a non-equimolar or equimolar mixture of diastereomers and/or enantiomers.

The compounds are particularly suitable for the treatment of neuropathic pain, in particular diabetic polyneuropathic pain or pain in postzosteric neuralgia. Substituted 4-aminocyclohexane derivatives according to the invention can accordingly be used for the preparation of a medicinal product for the treatment of pain, but particularly neuropathic or chronic pain, in particular diabetic polyneuropathic pain or pain in postzosteric neuralgia.

The invention therefore also provides the use of a substituted 4-aminocyclohexane derivative according to the invention for the preparation of a medicinal product for the treatment of pain, in particular neuropathic or chronic pain, in particular diabetic polyneuropathic pain or pain in postzosteric neuralgia.

The invention also provides the use of a substituted 4-aminocyclohexane derivative according to the invention to prepare a medicinal product for the treatment of anxiety conditions, stress and stress-related syndromes, depression, epilepsy, Alzheimer's disease, senile dementia, general cognitive dysfunctions, learning and memory disorders (as a nootropic), withdrawal symptoms, alcohol and/or drug and/or prescription drug abuse and/or dependency, sexual dysfunctions, cardiovascular diseases, hypotension, hypertension, tinnitus, pruritus, migraine, hearing impairment, gastrointestinal motility disorders, food intake disorders, anorexia, obesity, locomotive disorders, diarrhoea, cachexia, urinary incontinence, or as a muscle relaxant, anticonvulsant or anaesthetic, or for coadministration in treatment with an opioid analgesic or with an anaesthetic, for diuresis or antinatriuresis, anxiolysis, for the modulation of motor activity, for the modulation of neurotransmitter release and treatment of associated neurodegenerative diseases, for the treatment of withdrawal symptoms and/or for the reduction of the addiction potential of opioids.

In one of the above uses it can be preferable for a substituted 4-aminocyclohexane derivative that is used to be in the form of a pure diastereomer and/or enantiomer, a racemate or a non-equimolar or equimolar mixture of diastereomers and/or enantiomers.

The invention also provides a method for the treatment, in particular in one of the aforementioned indications, of a non-human mammal or human requiring treatment of pain, in particular neuropathic pain, by administration of a therapeutically active dose of a substituted 4-aminocyclohexane derivative according to the invention or of a medicinal product according to the invention.

The present invention also provides a method for preparing the substituted 4-aminocyclohexane compounds according to the invention. The chemicals and reaction components used in the reactions described are available commercially or can be produced by methods known to the person skilled in the art.

### General Method for Preparing Compounds Having the General Formula I

In methods I and II the ketal-protected 4-hydroxymethyl cyclohexanone 2 is used, which can be obtained from the commercially available 4-cyclohexanone carboxylic acid ethyl ester 1 by reaction with ethylene glycol in the presence of Brønsted acids, for example camphor sulfonic acid, p-toluenesulfonic acid or solid acids such as acid ion-exchange resins in relatively high-boiling organic solvents, for example in benzene, toluene or xylenes, at temperatures of between 20 and 130° C., and subsequent reaction with a reducing agent, for example a hydride such as sodium or lithium boron hydride, sodium cyanoboron hydride, sodium triacetoxyboron hydride, diisobutylaluminium hydride, lithium-tri-(sec-butyl)boron hydride (L-Selectride®) or lithiumaluminium hydride, optionally in the presence of Lewis acids, for example ZnCl₂, Ni(OAc)₂ or CoCl₂, in organic solvents, for example ether, diisopropylether, tetrahydrofuran or methyl-t-butyl ether, at temperatures of between 0 and 100° C.

### Method I:

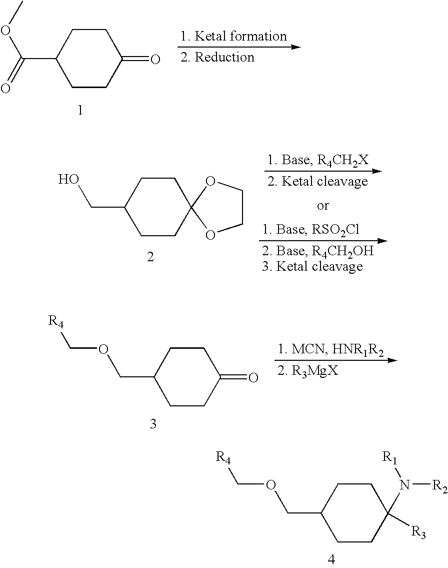

The reaction of the ketal-protected 4-hydroxymethyl cyclohexanone 2 to substituted cyclohexanones having the general formula 3 can take place in organic solvents, for example ether, tetrahydrofuran, dimethyl formamide, methanol, ethanol or dichloromethane, in the presence of an inorganic or organic base, for example potassium tert-butanolate, sodium methanolate or ethanolate, lithium or sodium hydride, potassium or sodium hydroxide, Proton Sponge, butyl lithium or other basic organometallic compounds such as Grignard reagents, for example ethyl magnesium chloride or bromide, optionally in the presence of phase-transfer catalysts, for example tetra-n-butylammonium chloride, tetra-n-butylammonium hydroxide or tetra-n-butylammonium iodide, and with an alkylating agent

R⁴CH₂X where X=Cl, Br

at temperatures of between −10 and 120° C. A ketal cleavage can then take place in an aqueous medium or organic solvents, for example tetrahydrofuran, acetone or butanone, or mixtures of these two media, in the presence of an inorganic acid, for example HCl, HBr, H₂SO₄, HClO₄ or H₃PO₄, or an organic acid, for example trifluoromethanoic acid, methanesulfonic acid, trifluoroacetic acid, camphor sulfonic acid, p-toluenesulfonic acid or pyridinium tosylate, optionally in the presence of PdCl₂, Pd(OAc)₂, PdCl₂(MeCN)₂ or PdCl₂(PPh₃)₂, at temperatures of between 20 and 80° C.

Alternatively the reaction of the ketal-protected 4-hydroxymethyl cyclohexanone 2 to substituted cyclohexanones having the general formula 3 can take place in organic solvents, for example ether, tetrahydrofuran, dimethyl formamide, methanol, ethanol or dichloromethane, in the presence of an organic base, for example Hünig's base, N-methyl piperidine, pyridine, triethylamine or N-methyl morpholine, and a sulfonic acid chloride, for example p-toluenesulfonic acid chloride, methanesulfonic acid chloride or trifluoromethanesulfonic acid chloride, at temperatures of between 20 and 80° C. The intermediate that is obtained can be reacted with a mixture of an alcohol

R⁴CH₂OH

in organic solvents, for example ether, tetrahydrofuran, dimethyl formamide, methanol, ethanol or dichloromethane, in the presence of an inorganic or organic base, for example potassium tert-butanolate, sodium methanolate or ethanolate, lithium or sodium hydride, potassium or sodium hydroxide, Proton Sponge, butyl lithium or other basic organometallic compounds such as Grignard reagents, for example ethyl magnesium chloride or bromide, optionally in the presence of phase-transfer catalysts, for example tetra-n-butylammonium chloride, tetra-n-butylammonium hydroxide or tetra-n-butylammonium iodide, at temperatures of between −10 and 120° C. A ketal cleavage can then take place in an aqueous medium or organic solvents, for example tetrahydrofuran, acetone or butanone, or mixtures of these two media, in the presence of an inorganic acid, for example HCl, HBr, H₂SO₄, HClO₄ or H₃PO₄, or an organic acid, for example trifluoromethanoic acid, methanesulfonic acid, trifluoroacetic acid, camphor sulfonic acid, p-toluenesulfonic acid or pyridinium tosylate, optionally in the presence of PdCl₂, Pd(OAc)₂, PdCl₂(MeCN)₂ or PdCl₂(PPh₃)₂, at temperatures of between 20 and 80° C.

The reaction of the substituted cyclohexanones having the general formula 3 to the monosubstituted 4-hydroxymethyl cyclohexylamine derivatives 4 according to the invention can take place in an aqueous medium or organic solvents, for example methanol, ethanol, n-propanol, tetrahydrofuran, acetonitrile or dichloromethane, or mixtures of these two media, in the presence of an inorganic acid, for example HCl, HBr, H₂SO₄, HClO₄ or H₃PO₄, a cyanide source, for example sodium or potassium cyanide, zinc cyanide, trimethylsilyl cyanide, acetone cyanohydrin, and an amine

HNR¹R²

or its hydrochloride

HCl.HNR¹R²

at temperatures of between 20 and 60° C. The intermediate that is obtained can then be reacted in organic solvents, for example hexane, pentane, toluene, ether, diisopropyl ether, tetrahydrofuran or methyl-t-butyl ether, with a Grignard reagent

MgXR³ where X=Cl, Br, I

at temperatures of between −10° C. and 40° C.

### Method II:

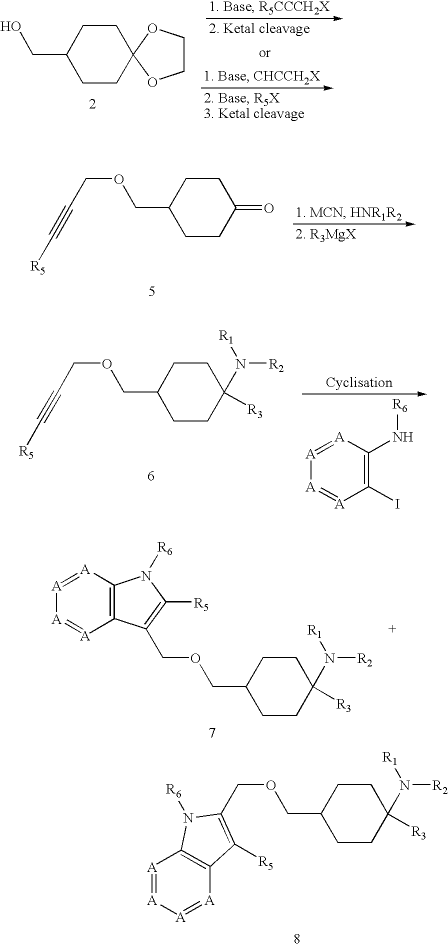

The reaction of the ketal-protected 4-hydroxymethyl cyclohexanone 2 to substituted cyclohexanones having the general formula 5 can take place in organic solvents, for example ether, tetrahydrofuran, dimethyl formamide, methanol, ethanol or dichloromethane, in the presence of an inorganic or organic base, for example potassium tert-butanolate, sodium methanolate or ethanolate, lithium or sodium hydride, potassium or sodium hydroxide, Proton Sponge, butyl lithium or other basic organometallic compounds such as Grignard reagents, for example ethyl magnesium chloride or bromide, optionally in the presence of phase-transfer catalysts, for example tetra-n-butylammonium chloride, tetra-n-butylammonium hydroxide or tetra-n-butylammonium iodide, and with an alkylating agent

R⁵CCCH₂X where X=Cl, Br

at temperatures of between −10 and 120° C. A ketal cleavage can then take place in an aqueous medium or organic solvents, for example tetrahydrofuran, acetone or butanone, or mixtures of these two media, in the presence of an inorganic acid, for example HCl, HBr, H₂SO₄, HClO₄ or H₃PO₄, or an organic acid, for example trifluoromethanoic acid, methanesulfonic acid, trifluoroacetic acid, camphor sulfonic acid, p-toluenesulfonic acid or pyridinium tosylate, optionally in the presence of PdCl₂, Pd(OAc)₂, PdCl₂(MeCN)₂ or PdCl₂(PPh₃)₂, at temperatures of between 20 and 80° C.

Alternatively the reaction of the ketal-protected 4-hydroxymethyl cyclohexanone 2 to substituted cyclohexanones having the general formula 5 can take place in organic solvents, for example ether, tetrahydrofuran, dimethyl formamide, methanol, ethanol or dichloromethane, in the presence of an inorganic or organic base, for example potassium tert-butanolate, sodium methanolate or ethanolate, lithium or sodium hydride, potassium or sodium hydroxide, Proton Sponge, butyl lithium or other basic organometallic compounds such as Grignard reagents, for example ethyl magnesium chloride or bromide, optionally in the presence of phase-transfer catalysts, for example tetra-n-butylammonium chloride, tetra-n-butylammonium hydroxide or tetra-n-butylammonium iodide, and with an alkylating agent

HCCCH₂X where X=Cl, Br, OSO₂CH₃, OSO₂-p-toluene

at temperatures of between −10 and 120° C. The intermediate that is obtained can be reacted in organic solvents, for example hexane, pentane, ether, diisopropyl ether, tetrahydrofuran or methyl-t-butyl ether, in the presence of a base, for example sodium amide, lithium diisopropylamide, butyl lithium, or other basic organometallic compounds, such as Grignard reagents, for example ethyl magnesium chloride or bromide, with electrophiles

R⁵X where X=Cl, Br, I

at temperatures of between −80 and 20° C.

A ketal cleavage can then take place in an aqueous medium or organic solvents, for example tetrahydrofuran, acetone or butanone, or mixtures of these two media, in the presence of an inorganic acid, for example HCl, HBr, H₂SO₄, HClO₄ or H₃PO₄, or an organic acid, for example trifluoromethanoic acid, methanesulfonic acid, trifluoroacetic acid, camphor sulfonic acid, p-toluenesulfonic acid or pyridinium tosylate, optionally in the presence of PdCl₂, Pd(OAc)₂, PdCl₂(MeCN)₂ or PdCl₂(PPh₃)₂, at temperatures of between 20 and 80° C.

The reaction of the substituted cyclohexanones having the general formula 5 to the monosubstituted 4-alkynyloxymethyl cyclohexylamine derivatives having the general formula 6 can take place in an aqueous medium or organic solvents, for example methanol, ethanol, n-propanol, tetrahydrofuran, acetonitrile or dichloromethane, or mixtures of these two media, in the presence of an inorganic acid, for example HCl, HBr, H₂SO₄, HClO₄ or H₃PO₄, a cyanide source, for example sodium or potassium cyanide, zinc cyanide, trimethylsilyl cyanide, acetone cyanohydrin, and an amine

HNR¹R²

or its hydrochloride

HCl.HNR¹R²

at temperatures of between 20 and 60° C. The intermediate that is obtained can then be reacted in organic solvents, for example hexane, pentane, toluene, ether, diisopropyl ether, tetrahydrofuran or methyl-t-butyl ether, with a Grignard reagent

MgXR³ where X=Cl, Br, I

at temperatures of between −10° C. and 40° C.

The reaction of the monosubstituted 4-alkynyloxymethyl cyclohexylamine derivatives having the general formula 6 to the monosubstituted 4-hydroxymethyl cyclohexylamine derivatives according to the invention having the general formulae 7 and 8 can take place in organic solvents, for example tetrahydrofuran, dimethyl formamide, benzene, toluene, xylenes, dimethoxyethane or diethylene glycol dimethyl ether, in the presence of an inorganic base, for example sodium, potassium or caesium carbonate or potassium phosphate, in the presence of PdCl₂, Pd(OAc)₂, PdCl₂(MeCN)₂, PdCl₂(PPh₃)₂ or [1,3-bis-(2,6-diisopropylphenyl)imidazol-2-ylidene]-(3-chloropyridyl)palladium(II) chloride (PEPPSI®), optionally in the presence of additional ligands, for example triphenyl, tri-o-tolyl, tricyclohexyl or tri-t-butyl phosphine, optionally in the presence of phase-transfer catalysts, for example tetra-n-butylammonium chloride, tetra-n-butylammonium hydroxide or tetra-n-butylammonium iodide, at temperatures of between 60° C. and 180° C., also with microwave assistance. Products 7 and 8 can be obtained separately or as mixtures and can optionally be purified by column chromatography.

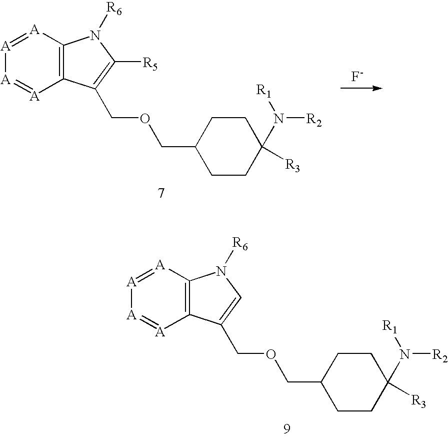

The reaction of the monosubstituted 4-hydroxymethyl cyclohexylamine derivatives having the general formula 7 to the monosubstituted 4-hydroxymethyl cyclohexylamine derivatives according to the invention having the general formula 9 can take place in organic solvents, for example hexane, pentane, ether, diisopropyl ether, tetrahydrofuran, methyl-t-butyl ether, acetonitrile, dimethyl formamide or dichloromethane, in the presence of a fluoride source, for example tetra-n-butylammonium fluoride, tetramethylammonium fluoride, benzyltrimethylammonium fluoride or HF pyridine, optionally in the presence of dehydrating reagents, for example molecular sieve 4 A, at temperatures of between 20° C. and 80° C.

### Method III:

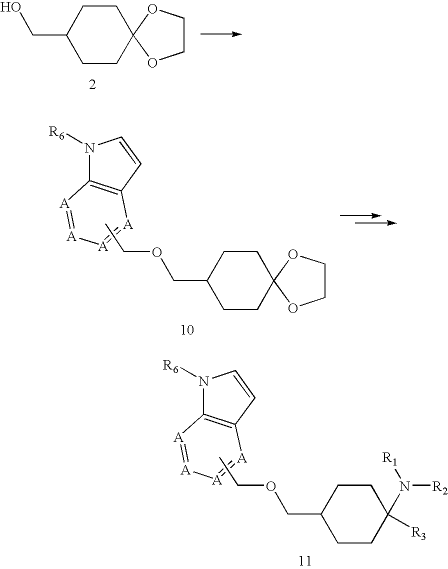

The reaction of the ketal-protected 4-hydroxymethyl cyclohexanone 2 to substituted cyclohexanones having the general formula 10 can take place in organic solvents, for example ether, tetrahydrofuran, dimethyl formamide, methanol, ethanol or dichloromethane, in the presence of an inorganic or organic base, for example potassium tert-butanolate, sodium methanolate or ethanolate, lithium or sodium hydride, potassium or sodium hydroxide, Proton Sponge, butyl lithium or other basic organometallic compounds such as Grignard reagents, for example ethyl magnesium chloride or bromide, optionally in the presence of phase-transfer catalysts, for example tetra-n-butylammonium chloride, tetra-n-butylammonium hydroxide or tetra-n-butylammonium iodide, and with an alkylating agent

R⁴CH₂X where X=Cl, Br

at temperatures of between −10 and 120° C.

The additional steps to obtain the monosubstituted 4-hydroxymethyl cyclohexylamine derivatives according to the invention having the general formula II can then take place in an analogous manner to methods I and II.

The following examples are intended to illustrate the invention without however restricting its scope.

### EXAMPLES

### 1.) Synthesis of the Structural Units

### Nitrile structural unit 1: 4-((3-(tert-Butyldimethylsilyl)prop-2-ynyloxy)methyl)-1-(dimethylamino)cyclohexane carbonitrile (Nit-01)

### Stage 1: 1,4-Dioxaspiro[4.5]decane-8-carboxylic acid ethyl ester

A solution of 4-oxocyclohexane carboxylic acid ethyl ester (27.6 g, 162 mmol), ethylene glycol (35.3 g, 31.7 ml, 569 mmol) and p-toluenesulfonic acid (360 mg, 1.89 mmol) in toluene (80 ml) was stirred for 20 h at room temperature. The reaction solution was then poured into diethyl ether (150 ml) and washed with water, 5% sodium hydrogen carbonate solution and saturated sodium chloride solution (150 ml each). The organic phase was dried with sodium sulfate and concentrated to small volume under vacuum.

Yield: 33 g (95%), colourless oil

¹H-NMR (DMSO-d₆): 1.17 (t, 3H, J=7.3 Hz); 1.41-1.69 (m, 6H); 1.77-1.82 (m, 1H); 2.28-2.40 (m, 2H); 3.84 (s, 4H); 4.04 (dd, 2H, J=6.8, 14.6 Hz).

### Stage 2: (1,4-Dioxaspiro[4.5]dec-8-yl)methanol

A solution of 1,4-dioxaspiro[4.5]decane-8-carboxylic acid ethyl ester (9.72 g, 45.4 mmol) in anhydrous tetrahydrofuran (50 ml) was added dropwise under argon at 65° C. to a suspension of lithium aluminium hydride (3.50 g, 93.3 mmol) in anhydrous tetrahydrofuran (100 ml) and then refluxed whilst stirring for 3 h. After adding water (6 ml) and 4 N sodium hydroxide solution (1.5 ml) the precipitate was filtered off and washed with tetrahydrofuran. The filtrate was dried with sodium sulfate and concentrated to small volume under vacuum.

Yield: 6.53 g (84%), colourless oil

¹H-NMR (DMSO-d₆): 1.03-1.18 (m, 2H); 1.30-1.18 (m, 3H); 1.59-1.70 (m, 4H); 3.21 (t, 2H, J=6.0 Hz); 3.83 (s, 4H); 4.40 (t, 1H, J=5.3 Hz).

¹³C-NMR (DMSO-d₆): 26.4; 33.8; 38.7; 63.4; 63.5; 66.7; 108.3.

### Stage 3: 8-((Prop-2-ynyloxy)methyl)-1,4-dioxaspiro[4.5]decane

A 60% dispersion of sodium hydride (1.40 g, 36.7 mmol) in mineral oil was added to a solution of (1,4-dioxaspiro[4.5]dec-8-yl)methanol (5.24 g, 30.4 mmol) in anhydrous tetrahydrofuran (150 ml) that had been cooled to 0° C., and the mixture was refluxed for 90 minutes. The reaction solution was then cooled to 0° C. and tetra-n-butylammonium iodide (241 mg, 0.63 mmol) and an 80% solution of propargyl bromide in toluene (10.8 g, 8.12 ml, 91.2 mmol) were added in succession, and the mixture was stirred overnight at room temperature. The reaction was then concentrated to small volume under vacuum, water (100 ml) was added to the residue and it was extracted with dichloromethane (3×80 ml). The combined organic phases were dried with sodium sulfate and concentrated to small volume under vacuum. The crude product (6.75 g) was purified by flash chromatography (100 g, 20×4.0 cm) with ethyl acetate/cyclohexane (1:9).

Yield: 4.31 g (67%), oil

¹H-NMR (DMSO-d₆): 1.10-1.24 (m, 2H); 1.42 (dt, 2H, J=12.6, 3.7 Hz); 1.49-1.60 (m, 1H); 1.65 (d, 4H, J=10.1 Hz); 3.26 (d, 2H, J=6.3 Hz); 3.39 (t, 1H, J=2.3 Hz); 3.83 (s, 4H); 4.09 (d, 2H, J=2.4 Hz).

### Stage 4: (3-(1,4-Dioxaspiro[4.5]decan-8-ylmethoxy)prop-1-ynyl)(tert-butyl)dimethylsilane

A 2.5 M solution of n-butyl lithium in n-hexane (8.5 ml, 21.2 mmol) was added dropwise under argon at −75° C. to a solution of 8-((prop-2-ynyloxy)methyl)-1,4-dioxaspiro[4.5]decane (4.30 g, 20.4 mmol) in anhydrous tetrahydrofuran (150 ml). The reaction mixture was heated to 0° C. and stirred at this temperature for 1 hour. After cooling again to −75° C. a solution of tert-butyldimethylchlorosilane (3.16 g, 21 mmol) in anhydrous tetrahydrofuran (50 ml) was added dropwise. The reaction mixture was then heated to room temperature and stirred overnight. After adding saturated ammonium chloride solution (35 ml) the suspension was extracted with diethyl ether (3×100 ml), the combined organic phases were washed with saturated sodium chloride solution (50 ml), dried with sodium sulfate and concentrated to small volume under vacuum.

Yield: 6.08 g (92%), brownish oil

¹H-NMR (DMSO-d₆): 0.09 (s, 6H); 0.91 (s, 9H); 1.10-1.22 (m, 2H); 1.35-1.47 (m, 2H); 1.50-1.60 (m, 1H); 1.64 (d, 4H, J=10.6 Hz); 3.28 (d, 2H, J=6.3 Hz); 3.83 (s, 4H); 4.13 (s, 2H).

### Stage 5: 4-((3-(tert-Butyldimethylsilyl)prop-2-ynyloxy)methyl)cyclohexanone

1 N Hydrochloric acid (15 ml) was added to a solution of (3-(1,4-dioxaspiro[4.5]decan-8-ylmethoxy)prop-1-ynyl)(tert-butyl)dimethylsilane (1.55 g, 4.77 mmol) in acetone (80 ml) and the mixture was stirred for 3 h at room temperature. 1 N sodium hydroxide solution (16 ml) was then added to the reaction mixture and it was extracted with dichloromethane (3×50 ml). The combined organic phases were dried with sodium sulfate and concentrated to small volume under vacuum. The crude product (1.30 g) was purified by flash chromatography (100 g, 20×4.0 cm) with ethyl acetate/cyclohexane (1:9).

Yield: 1.01 g (75%), colourless oil

¹H-NMR (DMSO-d₆): 0.09 (s, 6H); 0.92 (s, 9H); 1.66-1.44 (m, 2H); 1.90-2.10 (m, 3H); 2.14-2.23 (m, 2H); 2.37 (dt, 2H, J=13.9, 5.9 Hz); 3.37 (d, 2H, J=6.3 Hz); 4.17 (s, 2H).

¹³C-NMR (DMSO-d₆): −4.8; 16.0; 25.8; 28.8; 35.1; 39.6; 58.2; 73.0; 88.4; 103.5; 210.6.

### Stage 6: 4-((3-(tert-Butyldimethylsilyl)prop-2-ynyloxy)methyl)-1-(dimethylamino)cyclohexane carbonitrile

40% aqueous dimethylamine solution (11.7 ml, 88.9 mmol) followed by a solution of 4-((3-(tert-butyldimethylsilyl)prop-2-ynyloxy)methyl)cyclohexanone (5.19 g, 18.5 mmol) in methanol (20 ml) were added to a solution of 4 N hydrochloric acid (4.9 ml) and methanol (2.9 ml) that had been cooled to 0° C. Potassium cyanide (2.90 g, 44.4 mmol) was added to this mixture and it was stirred at room temperature for 5 days. After adding water (30 ml) it was extracted with diethyl ether (3×50 ml). The combined organic phases were dried with sodium sulfate and concentrated to small volume under vacuum.

Yield: 5.82 g (94%), brownish oil

¹H-NMR (DMSO-d₆): 0.088 and 0.09 (2 s, 6H); 0.09 and 0.92 (2 s, 9H); 1.05-1.21 (m, 2H); 1.26-1.57 (m, 3H); 1.63-1.83 (m, 2H); 2.20 (s, 2H); 2.24 (s, 6H); 3.27-3.32 (m, 2H); 4.13 and 4.15 (2 s, 2H).

The isomer ratio is approximately 2:3.

### Nitrile structural unit 2: 1-(Dimethylamino)-4-((3-(triethylsilyl)prop-2-ynyloxy)methyl)cyclohexane carbonitrile (Nit-02)

### Stage 1: (3-(1,4-Dioxaspiro[4.5]decan-8-ylmethoxy)prop-1-ynyl)triethylsilane

A 2.5 M solution of n-butyl lithium in n-hexane was added dropwise at −75° C. to a solution of 8-((prop-2-ynyloxy)methyl)-1,4-dioxaspiro[4.5]decane (stage 3, nitrile structural unit 1) (6.1 g, 29.0 mmol) in anhydrous tetrahydrofuran (150 ml). The mixture was stirred for one hour at 0° C. The solution was then cooled to −75° C. and a solution of chlorotriethylsilane (4.5 g, 29.8 mmol) in anhydrous tetrahydrofuran (50 ml) was added dropwise. The mixture was stirred overnight at room temperature, then saturated ammonium chloride solution (50 ml) was added dropwise and it was extracted with diethyl ether (3×70 ml). The combined organic phases were dried with sodium sulfate and concentrated to small volume under vacuum.

Yield: 8.92 g (95%), brown oil

¹H-NMR (DMSO-d₆): 0.57 (q, 6H, J=7.9, 8.1 Hz); 0.95 (t, 9H, J=7.8 Hz); 1.10-1.24 (m, 2H); 1.42 (dd, 2H, J=9.3, 12.5 Hz); 1.52-1.60 (m, 1H); 1.64 (d, 4H, J=10.6 Hz); 3.29 (d, 2H, J=6.3 Hz); 3.83 (s, 4H); 4.13 (s, 2H).

### Stage 2: 4-((3-(Triethylsilyl)prop-2-ynyloxy)methyl)cyclohexanone

A 1 M hydrochloric acid solution (70 ml) was added to a solution of (3-(1,4-dioxaspiro[4.5]decan-8-ylmethoxy)prop-1-ynyl)triethylsilane (8.40 g, 25.9 mmol) in acetone (160 ml) and the mixture was stirred at room temperature for 6 hours. The reaction mixture was then extracted with dichloromethane (3×25 ml). The combined organic phases were dried with sodium sulfate and concentrated to small volume under vacuum. The crude product (7.24 g) was purified by flash chromatography (400 g, 7.6×20 cm) with cyclohexane/ethyl acetate (8.5:1.5).

Yield: 5.10 g (66%), yellowish oil

¹H-NMR (DMSO-d₆): 0.58 (q, 6H, J=7.9, 8.1 Hz); 0.96 (t, 9H, J=7.8 Hz); 1.36 (dq, 2H, J=ca. 4, 13.0 Hz); 1.92-2.10 (m, 3H); 2.19 (br d, 2H, J=14.4 Hz); 2.37 (dt, 2H, J=5.8, 13.5 Hz); 3.38 (d, 2H, J=6.3 Hz); 4.17 (s, 2H).

### Stage 3 (Method 1): 1-(Dimethylamino)-4-((3-(triethylsilyl)prop-2-ynyloxy)methyl)cyclohexane carbonitrile (Nit-02)

40% aqueous dimethylamine solution (26.4 ml, 192 mmol) followed by 4-((3-(triethylsilyl)prop-2-ynyloxy)methyl)cyclohexanone (12.1 g, 43.1 mmol) in methanol (50 ml) were added to a solution of 4 N hydrochloric acid (10.8 ml) and methanol (12 ml) that had been cooled to 0° C. Potassium cyanide (6.81 g, 102 mmol) was added to this mixture and it was stirred at room temperature for 2 hours. After adding water (200 ml) it was extracted with diethyl ether (3×60 ml). The combined organic phases were dried with sodium sulfate and concentrated to small volume under vacuum.

Yield: 12.7 g (88%), yellowish oil

¹H-NMR (DMSO-d₆): 0.57 and 0.78 (2 q, 6H, J=7.8 Hz); 0.95 and 0.96 (2 t, 9H, J=7.8 Hz); 1.04-1.50 (m, 5H); 1.65-1.85 (m, 3H); 2.07 (br d, 1H, J=13.6 Hz); 2.20 and 2.24 (2 s, 6H); 3.29 (d, 1H, J=6.4 Hz); 3.32 (d, 1H, J=6.2 Hz); 4.13 and 4.15 (2 s, 2H).

### Stage 3 (method 2): 1-(Dimethylamino)-4-((3-(triethylsilyl)prop-2-ynyloxy)methyl)cyclohexane carbonitrile (Nit-02)

A solution of 4 N hydrochloric acid (1.1 ml), methanol (0.6 ml) and 40% aqueous dimethylamine solution (2.5 ml, 19.2 mmol) that had been cooled to 0° C. was added to 4-((3-(triethylsilyl)prop-2-ynyloxy)methyl)cyclohexanone (1.12 mg, 4 mmol). Potassium cyanide (624 mg, 9.6 mmol) was added to this mixture and it was stirred at room temperature for 5 days. After adding water (10 ml) it was extracted with diethyl ether (3×10 ml). The combined organic phases were dried with sodium sulfate and concentrated to small volume under vacuum.

Yield: 1.04 g (78%), colourless oil

### Nitrile structural unit 3: 4-But-2-ynyloxymethyl-1-dimethylaminocyclohexane carbonitrile (Nit-03) (mixture of diastereomers)

### Stage 1: 8-But-2-ynyloxymethyl-1,4-dioxaspiro[4.5]decane

A 60% dispersion of sodium hydride in mineral oil (2.1 g, 55 mmol) was added with ice cooling to a solution of (1,4-dioxaspiro[4.5]dec-8-yl)methanol (stage 2, nitrile structural unit 1) (8.6 g, 50 mmol) in anhydrous tetrahydrofuran (200 ml). The mixture was refluxed whilst stirring for 1.5 h then cooled to 0° C. and a solution of 1-bromobut-2-yne (19.2 g, 144 mmol) in anhydrous tetrahydrofuran (40 ml) was added dropwise. The mixture was stirred overnight at room temperature and the tetrahydrofuran then distilled off under vacuum. Water (140 ml) was added to the residue and it was extracted with dichloromethane (4×40 ml). The combined organic phases were dried with sodium sulfate and concentrated to small volume under vacuum. The crude product (15.0 g) was purified by flash chromatography (400 g, 20×7.6 cm) with cyclohexane/ethyl acetate (4:1).

Yield: 4.47 g (40%), yellowish oil

¹H-NMR (DMSO-d₆): 1.10-1.23 (m, 2H); 1.36-1.47 (m, 2H); 1.54 (m, 1H); 1.64 (d, 4H, J=10.2 Hz); 1.81 (t, 3H, J=2.3 Hz); 3.24 (d, 2H, J=6.4 Hz); 3.83 (s, 4H); 4.04 (q, 2H, J=2.3 Hz).

### Stage 2: 4-But-2-ynyloxymethylcyclohexanone

2 M Hydrochloric acid (56 ml) was added to a solution of 8-but-2-ynyloxymethyl-1,4-dioxaspiro[4.5]decane (4.75 g, 21.1 mmol) in acetone (150 ml) and the mixture was stirred at room temperature for 48 h. The acetone was then distilled off under vacuum, the residue made alkaline with 4 M sodium hydroxide solution and extracted with dichloromethane (3×30 ml). The combined organic phases were dried with sodium sulfate and concentrated to small volume under vacuum.

Yield: 3.59 g (94%), yellowish oil

¹H-NMR (DMSO-d₆): 1.26-1.44 (m, 2H); 1.82 (t, 3H, J=2.3 Hz); 1.90-2.02 (m, 3H); 2.14-2.24 (m, 2H); 2.37 (dt, 2H, J=13.6, 5.9 Hz); 3.33 (d, 2H, J=6.1 Hz); 4.08 (q, 2H, J=2.3 Hz).

### Stage 3: 4-But-2-ynyloxymethyl-1-dimethylaminocyclohexane carbonitrile (Nit-03) (mixture of diastereomers)

40% aqueous dimethylamine solution (12.12 ml) followed by a solution of 4-but-2-ynyloxymethylcyclohexanone (3.57 g, 19.8 mmol) in methanol (23.6 ml) were added to a mixture of 4 M hydrochloric acid (4.96 ml) and methanol (5.3 ml) that had been cooled to 0° C. Potassium cyanide (3.12 g, 46.8 mmol) was then added to this mixture and it was stirred overnight at room temperature. After adding water (90 ml) it was extracted with diethyl ether (3×30 ml). The combined organic phases were dried with sodium sulfate and concentrated to small volume under vacuum. The product is a mixture of diastereoisomers in the ratio of approximately 2:1.

Yield: 4.16 g (89%), yellowish oil

¹H-NMR (DMSO-d₆): 1.06-1.20 (m, 1.3H); 1.22-1.40 (m, 2H); 1.42-1.60 (m, 2.3H); 1.62-1.74 (m, 1H); 1.816 and 1.823 (2 t, 3H, J=2.4 Hz); 2.09 (m, 0.7H); 2.18-2.25 (m, 1.7H); 2.21 and 2.24 (2s, 6H); 3.23 (d, 0.7H, J=6.4 Hz); 3.27 (d, 1.3H, J=6.4 Hz); 4.04 (q, 0.7H, J=2.4 Hz); 4.06 (q, 1.3H, J=2.4 Hz).

### Nitrile structural unit 4: 1-(Dimethylamino)-4-((4,4-dimethylpent-2-ynyloxy)methyl)cyclohexane carbonitrile (Nit-04) (mixture of diastereomers)

### Stage 1: 4,4-Dimethylpent-2-yn-1-ol

A 2.5 M solution of n-butyl lithium (19.5 g, 122 ml, 304 mmol) in hexane was slowly added dropwise at 0° C. under argon to a solution of 3,3-dimethyl-1-butyne (25.0 g, 304 mmol) in absolute diethyl ether (125 ml). Absolute tetrahydrofuran (100 ml) was then added dropwise, the reaction solution was heated to room temperature, paraformaldehyde (11.5 g, 383 mmol) was added in portions over a period of 45 min, then the mixture was refluxed for 4 h whilst stirring and then stirred overnight at room temperature. The reaction mixture was poured into water (1 l), the phases were separated and the aqueous phase was extracted with diethyl ether (3×100 ml). The combined organic phases were washed with saturated ammonium chloride solution and saturated sodium chloride solution (100 ml each), dried with sodium sulfate and—owing to the low boiling point of the product (Kₚ=140° C. under normal pressure, 68-69° C. under 17 torr)—concentrated with care to small volume under vacuum.

Yield: 31.6 g (93%), yellow oil

¹H-NMR (DMSO-d₆): 1.17 (s, 9H); 4.01 (d, 2H, J=5.4 Hz); 5.00 (t, 1H, J=5.6 Hz).

### Stage 2: 1-Bromo-4,4-dimethylpent-2-yne

A solution of 4,4-dimethylpent-2-yn-1-ol (25.0 g, 223 mmol) in absolute acetonitrile (100 ml) was added dropwise under argon to a suspension of triphenylphosphine dibromide (107 g, 253 mmol) in absolute acetonitrile (750 ml) and imidazole (17.2 g, 253 mmol). The mixture was then stirred overnight at room temperature. Owing to the low boiling point of the product (Kₚ=60-64° C. under 20 torr), the mixture was concentrated to small volume under vacuum at room temperature. The residue was washed with pentane (1 l) and the filtrate was likewise concentrated to small volume under vacuum at room temperature. The residue still contained some solids and was therefore treated again with pentane (100 ml) in the manner described above. Since the product (14.7 g) was obtained in a very pure form, it was able to be reacted further directly.

Yield: 14.7 g (38%), pale yellow oil

¹H-NMR (DMSO-d₆): 1.18 (s, 9H); 4.19 (s, 2H).

### Stage 3: 8-(4,4-Dimethylpent-2-ynyloxymethyl)-1,4-dioxaspiro[4.5]decane

A solution of (1,4-dioxaspiro[4.5]dec-8-yl)methanol (3.67 g, 21.3 mmol) in absolute tetrahydrofuran (25 ml) was added dropwise with argon flushing to a 60% sodium hydride suspension in mineral oil (1.02 g, 25.5 mmol) in absolute tetrahydrofuran (50 ml) and the mixture was then refluxed for 90 minutes. After cooling to room temperature a solution of 1-bromo-4,4-dimethylpent-2-yne (11.2 g, 64.0 mmol) in absolute tetrahydrofuran (25 ml) was added dropwise and the mixture was then stirred over the weekend. The reaction solution was concentrated to small volume under vacuum, water (100 ml) was added and the suspension was extracted with ethyl acetate (3×50 ml). The combined organic phases were washed with saturated sodium chloride solution (50 ml), dried with sodium sulfate and concentrated to small volume under vacuum. The crude product (11.3 g) was purified by flash chromatography (400 g, 20×7.5 cm) with cyclohexane/ethyl acetate (9:1).

Yield: 1.70 g (30%), pale yellow oil

¹H-NMR (DMSO-d₆): 1.18 (s, 9H); 1.35-1.47 (m, 2H); 1.49-1.58 (m, 1H); 1.59-1.70 (m, 6H); 3.32 (s, 2H, J=6.3 Hz); 3.83 (s, 4H); 4.02 (s, 2H).

### Stage 4: 4-(4,4-Dimethylpent-2-ynyloxymethyl)cyclohexanone

1 N Hydrochloric acid (6 ml) was added to a solution of 8-(4,4-dimethylpent-2-ynyloxymethyl)-1,4-dioxaspiro[4.5]decane (1.70 g, 6.4 mmol) in acetone (30 ml) and the mixture was stirred overnight at room temperature. As some of the starting product could still be detected (LC-MS), the mixture was stirred for a further 24 h. The reaction solution was then adjusted to pH 8 with 1 N sodium hydroxide solution and concentrated to small volume under vacuum. Water (20 ml) was added to the residue and it was then extracted with dichloromethane (3×20 ml). The combined organic phases were washed with saturated sodium chloride solution (20 ml), dried with sodium sulfate and concentrated to small volume under vacuum. According to the ¹H-NMR spectrum the crude product (1.49 g) contained approximately 5% 8-(4,4-dimethylpent-2-ynyloxymethyl)-1,4-dioxaspiro[4.5]decane. It was reacted further without purification.

Yield: 1.49 g (100%), pale yellow oil

¹H-NMR (DMSO-d₆): 1.19 (s, 9H); 1.37-1.43 (m, 2H); 1.92-2.08 (m, 3H); 2.16-2.24 (m, 2H); 2.32-2.41 (m, 2H); 3.33 (d, 2H, J=6.3 Hz); 4.08 (s, 2H).

LC-MS: m/z: [M+1]⁺=223.2, Rₜ 3.4 min.

### Stage 5: 1-(Dimethylamino)-4-((4,4-dimethylpent-2-ynyloxy)methyl)cyclohexane carbonitrile (Nit-04) (mixture of diastereomers)

40% dimethylamine solution (1.64 ml, 26.6 mmol), followed by 4-(4,4-dimethylpent-2-ynyloxymethyl)cyclohexanone (1.49 g, 6.7 mmol) and potassium cyanide (1.05 g, 16.1 mmol) were added to an ice-cooled mixture of 4 N hydrochloric acid (1.68 ml, 6.7 mmol) and methanol (7 ml). The suspension formed was stirred at room temperature for 66 h. Water (50 ml) was added to the suspension and it was then extracted with diethyl ether (3×50 ml). The combined organic phases were dried with sodium sulfate and concentrated to small volume under vacuum. The crude product (1.54 g) was reacted further directly. The product is a mixture of diastereoisomers in the ratio 2:1.

Yield: 1.54 g (83%), pale yellow oil

¹H-NMR (DMSO-d₆): 1.18 and 1.19 (2s, 9H); 1.24-2.12 (m, 8H); 2.16-2.20 (m, 1H); 2.21 and 2.24 (2s, 6H); 3.23 and 3.27 (2d, 2H, J=6.3 Hz); 4.05 and 4.06 (2s, 2H).

### Triazole Structural Element 1

### 1-Dimethylamino-1-[1,2,3]triazol-1-yl-4-(3-triethylsilanylprop-2-ynyloxymethyl)cyclohexane (Tri-01)

A 2 M solution of dimethylamine in tetrahydrofuran (4.4 ml, 8.8 mmol), 1,2,3-triazole (540 mg, 7.8 mmol) and 4 Å molecular sieve (2.70 g) was added to a solution of 4-((3-(triethylsilyl)prop-2-ynyloxy)methyl)cyclohexanone (2.00 g, 7.1 mmol) in anhydrous tetrahydrofuran (50 ml). The mixture was stirred overnight at room temperature, then filtered and immediately reacted further.

Yield: 2.67 g (100%)

¹H-NMR (DMSO-d₆): 0.57 (q, 6H, J=7.7 Hz); 0.95 (t, 9H, J=7.8 Hz); 1.12-1.30 (m 1H); 1.55-1.82 (m, 4H); 1.90-2.25 (m, 4H); 2.47 (s, 6H); 3.27-3.41 (m, 2H); 4.15 (s, 2H); 6.87 (s, 1H); 7.84 (s, 1H).

### Alkyne Structural Unit 1 (Method 1):

### 1-Phenyl-4-((3-(triethylsilanyl)prop-2-ynyloxy)methyl)-N,N-dimethylcyclohexanamine (Ain-01; one of two diastereomers)

A solution of 1-(dimethylamino)-4-((3-(triethylsilyl)prop-2-ynyloxy)methyl)cyclohexane carbonitrile (Nit-02) (4.50 g, 13.4 mmol) in anhydrous tetrahydrofuran (40 ml) was added dropwise under argon and at 0° C. to a 2 M solution of phenyl magnesium chloride (16.8 ml, 33.6 mmol) in tetrahydrofuran and the mixture was then stirred overnight at room temperature. After adding saturated ammonium chloride solution and water (20 ml each) the phases were separated and the aqueous phase was extracted with diethyl ether (3×30 ml). The combined organic phases were dried with sodium sulfate and concentrated to small volume under vacuum. The crude product (5.00 g) was purified by flash chromatography (100 g, 20×4.0 cm) with ethyl acetate/cyclohexane (1:9) and the mixed fractions obtained (2.00 g) were purified by another flash chromatography step (100 g, 20×4.0 cm) with chloroform/isopropanol (300:1).

Yield: 2.67 g (52%), colourless oil

¹H-NMR (DMSO-d₆): 0.58 (q, 6H, J=7.8 Hz); 0.96 (t, 9H, J=7.8 Hz); 1.25-1.65 (m, 7H); 1.91 (s, 6H); 2.57 (br d, 2H, J=11.4 Hz); 3.35 (d, 2H, J=6.1 Hz); 4.15 (s, 2H); 7.21-7.38 (m, 5H).

Only one of the two possible diastereoisomers was isolated.

### Alkyne Structural Unit 1 (Method 2):

### 1-Phenyl-4-((3-(triethylsilanyl)prop-2-ynyloxy)methyl)-N,N-dimethylcyclohexanamine (Ain-01; one of two diastereomers)

A solution of 1-dimethylamino-1-[1,2,3]triazol-1-yl-4-(3-triethylsilanylprop-2-ynyloxymethyl)cyclohexane (Tri-01) (2.67 g, 7.1 mmol) in anhydrous tetrahydrofuran (50 ml) was added dropwise under argon and at 0° C. to a 2 M solution of phenyl magnesium chloride (8.9 ml, 18 mmol) in tetrahydrofuran and then stirred overnight at room temperature. After adding saturated ammonium chloride solution and water (10 ml each) the phases were separated and the aqueous phase was extracted with diethyl ether (3×30 ml). The organic phase was dried with sodium sulfate and concentrated to small volume under vacuum. The crude product (2.60 g) was purified by flash chromatography (100 g, 20×4.0 cm) with ethyl acetate/cyclohexane (1:9) and ethyl acetate/methanol (1:9).

Yield: 779 mg (28%), oil

¹H-NMR (DMSO-d₆): 0.58 (q, 6H, J=7.9 Hz); 0.96 (t, 9H, J=7.9 Hz); 1.30-1.54 (m, 7H); 1.91 (s, 6H); 2.58 (br d, 2H, J=11.3 Hz); 3.35 (d, 2H, J=6.2 Hz); 4.15 (s, 2H); 7.20-7.40 (m, 5H).

### Alkyne Structural Unit 2:

### 1-Phenyl-4-((3-(tert-butyldimethylsilyl)prop-2-ynyloxy)methyl)-N,N-dimethylcyclohexanamine (Ain-02; one of two diastereomers)

A solution of 4-((3-(tert-butyldimethylsilyl)prop-2-ynyloxy)methyl)-1-(dimethylamino)cyclohexane carbonitrile (Nit-01) (5.50 g, 16.4 mmol) in anhydrous tetrahydrofuran (80 ml) was added dropwise under argon at 0° C. to a 2 M solution of phenyl magnesium chloride (20.5 ml, 41 mmol) in tetrahydrofuran and the mixture was then stirred overnight at room temperature. After adding saturated ammonium chloride solution (15 ml) and water (15 ml) the phases were separated and the aqueous phase was extracted with diethyl ether (3×30 ml). The combined organic phases were dried with sodium sulfate and concentrated to small volume under vacuum. The crude product (5.73 g) was purified by flash chromatography (200 g, 20×5.7 cm) with ethyl acetate/cyclohexane (1:9→1:2).

Yield: 3.98 g (63%), yellowish oil

¹H-NMR (DMSO-d₆): 0.10 (s, 6H); 0.93 (s, 9H); 1.30-1.65 (m, 7H); 1.92 (s, 6H); 2.57 (d, 2H, J=12.4 Hz); 3.35 (d, 2H, J=6.3 Hz); 4.15 (s, 2H); 7.29-7.36 (m, 5H).

¹³C-NMR (DMSO-d₆): −4.8; 16.1; 24.4; 25.8; 32.0; 36.7, 37.4; 58.1; 58.5; 74.5; 88.2; 103.8; 126.1; 126.5; 127.2; 139.3.

### Alkyne Structural Units 4 & 7:

### 1-Benzyl-4-((3-(tert-butyldimethylsilyl)prop-2-ynyloxy)methyl)-N,N-dimethylcyclohexanamine (Ain-04; non-polar diastereomer and Ain-07; polar diastereomer)

A solution of 4-((3-(tert-butyldimethylsilyl)prop-2-ynyloxy)methyl)-1-(dimethylamino)cyclohexane carbonitrile (Nit-01) (5.10 g, 15.2 mmol) in tetrahydrofuran (25 ml) was added dropwise under argon with ice cooling to a 2 M solution of benzyl magnesium chloride in tetrahydrofuran (22.8 ml, 45.6 mmol) in a heated flask. The mixture was stirred at room temperature for 20 h and then 20% ammonium chloride solution (20 ml) was added with iced water cooling and the mixture was extracted with diethyl ether (3×50 ml). The combined organic phases were washed with saturated sodium chloride solution, dried with sodium sulfate and concentrated to small volume under vacuum. The crude product (6 g) was purified by flash chromatography (360 g, 20×7.5 cm) with ethyl acetate/cyclohexane (1:4→1:2→1:1) and then with methanol/25% aqueous ammonia solution (9:1). The polar diastereoisomer was purified again by flash chromatography (90 g, 20×4 cm) with ethyl acetate/methanol (4:1).

### Ain-04, Non-Polar Diastereoisomer

Yield: 2.25 g (37%), colourless oil

¹H-NMR (DMSO-d₆): 0.05 (s, 6H); 0.88 (s, 9H); 0.90-1.01 (m, 2H); 1.16-1.34 (m, 5H); 1.78 (d, J=13.0 Hz, 2H); 2.22 (s, 6H); 2.56 (s, 2H); 3.20 (d, J=4.4 Hz, 2H); 4.06 (s, 2H); 7.09-7.27 (m, 5H).

### Ain-07, Polar Diastereomer Yield: 1.5 g (25%), Colourless Oil

¹H-NMR (DMSO-d₆): 0.08 (s, 6H); 0.91 (s, 9H); 1.14-1.77 (m, 9H); 2.25 (s, 6H); 2.71 (br s, 2H); 3.25 (d, J=6.5 Hz, 2H); 4.09 (s, 2H); 7.12-7.33 (m, 5H).

### Alkyne Structural Units 5 & 6:

### 1-Benzyl-N,N-dimethyl-4-((3-(triethylsilyl)prop-2-ynyloxy)methyl)cyclohexanamine (Ain-05, non-polar diastereomer and Ain-06, polar diastereomer)

A solution of 1-(dimethylamino)-4-((3-(triethylsilyl)prop-2-ynyloxy)methyl)cyclohexane carbonitrile (Nit-02) (5.0 g, 14.9 mmol) in tetrahydrofuran (25 ml) was added dropwise under argon with ice cooling to a 2 M solution of benzyl magnesium chloride in tetrahydrofuran (22.4 ml, 44.8 mmol) and the mixture was stirred at room temperature for 20 h. 20% ammonium chloride solution (20 ml) was then added to the reaction mixture with iced water cooling. The phases were separated and the aqueous phase was extracted with diethyl ether (3×50 ml). The combined organic phases were washed with saturated sodium chloride solution, dried with sodium sulfate and concentrated to small volume under vacuum. The crude product (6.8 g) was purified by flash chromatography (250 g, 23×5.5 cm) with ethyl acetate/cyclohexane (1:4→1:1→1:0) and then with ethyl acetate/methanol (4:1). The polar diastereoisomer was purified again by flash chromatography (40 g, 9×4 cm) with ethyl acetate/methanol (4:1).

### Ain-05 (Non-Polar Diastereomer)

Yield: 2.08 g (35%), colourless oil

¹H-NMR (DMSO-d₆): 0.54 (q, J=8.2, 7.9 Hz, 6H); 0.92 (t, J=7.9 Hz, 9H); 0.92-1.01 (m, 2H); 1.17-1.34 (m, 5H); 1.78 (d, J=12.9 Hz, 2H); 2.22 (s, 6H); 2.56 (s, 2H); 3.21 (d, J=4.7 Hz, 2H); 4.07 (s, 2H); 7.09-7.27 (m, 5H).

### Ain-06 (Polar Diastereomer)

Yield: 1.78 g (30%), colourless oil

¹H-NMR (DMSO-d₆): 0.56 (q, J=7.8 Hz, 6H); 0.94 (t, J=7.8 Hz, 9H); 1.16-1.35 (m, 4H); 1.40-1.53 (m, 2H); 1.55-1.73 (m, 3H); 2.21 (s, 6H); 2.68 (s, 2H); 3.25 (d, J=6.4 Hz, 2H); 4.09 (s, 2H); 7.10-7.27 (m, 5H).

### Alkyne Structural Unit 8:

### 1-Butyl-N,N-dimethyl-4-((3-(triethylsilyl)prop-2-ynyloxy)methyl)cyclohexanamine (Ain-08, non-polar diastereomer)

A solution of Nit-02 (5.02 g, 15.0 mmol) in anhydrous tetrahydrofuran (80 ml) was added dropwise with ice cooling to a 2 M solution of n-butyl magnesium chloride in tetrahydrofuran (18.7 ml, 37.5 mmol) and the mixture was stirred overnight at room temperature. Saturated ammonium chloride solution (15 ml) and water (15 ml) were then added dropwise to the reaction mixture with ice cooling. The phases were separated and the aqueous phase was extracted with diethyl ether (3×30 ml). The combined organic phases were dried with sodium sulfate and concentrated to small volume under vacuum. The crude product (5.28 g) was purified by flash chromatography (420 g, 30×7.6 cm) with cyclohexane/ethyl acetate (4:1)→cyclohexane/ethyl acetate (1:1)→ethyl acetate/methanol (1:1).

### Ain-08, Non-Polar Diastereomer

Yield: 3.14 g (57%), yellowish oil

¹H-NMR (DMSO-d₆): 0.57 (q, 6H, J=7.9 Hz); 0.87 (t, 3H, J=7.1 Hz); 0.95 (t, 9H, J=7.8 Hz); 1.10-1.56 (m, 13H); 1.66 (d, 2H, J=11.8 Hz); 2.12 (s, 6H); 3.27 (d, 2H, J=6.3 Hz); 4.12 (s, 2H).

¹³C-NMR (DMSO-d₆): 3.7; 7.2; 13.9; 23.3; 23.9; 25.4; 26.3; 28.8; 30.6; 31.4; 35.8; 36.7; 37.1; 55.6; 58.1; 74.9; 87.2; 104.4.

### Alkyne Structural Unit 9:

### N,N-Dimethyl-1-(thiophen-2-yl)-4-((3-(triethylsilyl)prop-2-ynyloxy)methyl)cyclohexanamine (Ain-09; one of two possible diastereomers)

A solution of Nit-02 (2.12 g, 6.34 mmol) in anhydrous tetrahydrofuran (20 ml) was added dropwise under argon at 0° C. to a 1 M solution of 2-thienyl magnesium bromide in tetrahydrofuran (15.8 ml, 15.8 mmol) and the mixture was then stirred overnight at room temperature. After adding saturated ammonium chloride solution (5 ml) and water (10 ml) the phases were separated and the aqueous phase was extracted with diethyl ether (3×20 ml). The combined organic phases were dried with sodium sulfate and concentrated to small volume under vacuum. The crude product (2.40 g) was purified by flash chromatography (200 g, 20×5.7 cm) with ethyl acetate/cyclohexane (1:9).

Yield: 1.36 g (55%), brownish oil

¹H-NMR (DMSO-d₆): 0.53-0.63 (m, 6H); 0.92-1.00 (m, 9H); 1.33-1.70 (m, 7H); 1.99 (s, 6H); 2.39 (br d, 2H, J=11.9 Hz); 3.27-3.35 (m, 2H overlaid by the HDO signal); 4.15 (s, 2H); 6.91 (dd, 1H, J=3.5, 1.0 Hz); 7.03 (dd, 1H, J=5.1, 3.5 Hz); 7.39 (dd, 1H, J=5.1, 0.9 Hz).

¹³C-NMR (DMSO-d₆): 3.7; 7.2; 24.2; 34.6; 36.6; 37.4; 58.1; 58.2; 74.4; 87.3; 104.3; 123.0; 123.8; 126.1; 145.0.

LC-MS: (Method: ASCA-7 MIN-80 DEG.M): m/z: [M+H]⁺=392.3, Rₜ 3.5 min

### Alkyne Structural Unit 10:

### 1-(1-Phenyl-4-((3-(triethylsilyl)prop-2-ynyloxy)methyl)cyclohexyl)azetidine (Ain-10, one of two possible diastereomers)

### Stage 1: 1-(Azetidin-1-yl)-4-((3-(triethylsilyl)prop-2-ynyloxy)methyl)cyclohexane carbonitrile

Azetidine (2.18 g, 2.58 ml, 38.3 mmol) and then a solution of 4-((3-(triethylsilyl)prop-2-ynyloxy)methyl)cyclohexanone (2.24 g, 8 mmol) in methanol (5 ml) were added to a mixture of 4 N hydrochloric acid (2 ml) and methanol (1 ml) cooled to 0° C. A solution of potassium cyanide (1.24 g, 19.1 mmol) in water (5 ml) was added to this mixture and it was then stirred at room temperature for 3 h. After adding water (10 ml) to the reaction mixture it was extracted with diethyl ether (3×20 ml). The combined organic phases were dried with sodium sulfate and concentrated to small volume under vacuum.

Yield: 2.52 g (91%), yellowish oil

¹H-NMR (DMSO-d₆): 0.54-0.61 (m, 6H); 0.96 (t, 9H, J=7.8 Hz); 1.05-1.85 (m, 9H); 1.92-2.02 (m, 2H); 3.15-3.23 (m, 4H); 3.27-3.33 (m, 2H); 4.13 and 4.15 (2 s, 2H).

The product is a mixture of diastereoisomers.

### Stage 2: 1-(1-Phenyl-4-((3-(triethylsilyl)prop-2-ynyloxy)methyl)cyclohexyl)azetidine (Ain-10, one of two possible diastereomers)

A solution of 1-(azetidin-1-yl)-4-((3-(triethylsilyl)prop-2-ynyloxy)methyl)cyclohexane carbonitrile (2.52 g, 7.2 mmol) in anhydrous tetrahydrofuran (20 ml) was added dropwise under argon at 0° C. to a 2 M solution of phenyl magnesium chloride in tetrahydrofuran (9 ml, 18 mmol) and the mixture was then stirred overnight at room temperature. After adding saturated ammonium chloride solution (5 ml) and water (10 ml) the phases were separated and the aqueous phase was extracted with diethyl ether (3×20 ml). The combined organic phases were dried with sodium sulfate and concentrated to small volume under vacuum. The crude product (2.72 g) was purified by flash chromatography (100 g, 20×4.0 cm) with ethyl acetate/cyclohexane (1:9).

Yield: 1.76 g (61%), yellowish oil

¹H-NMR (DMSO-d₆): 0.53-0.63 (m, 6H); 0.92-1.00 (m, 9H); 1.25-1.35 (m, 2H); 1.45-1.53 (m, 5H); 1.61 (quin, 2H, J=7.1 Hz); 2.19 (br d, 2H, J=13.0 Hz); 2.83 (t, 4H, J=6.8 Hz); 3.34-3.40 (m, 2H); 4.16 (s, 2H); 7.22-7.42 (m, 5H).

¹³C-NMR (DMSO-d₆): 3.7; 7.2; 15.6; 24.7; 28.8; 30.3; 35.1; 36.4; 45.8; 57.2; 58.2; 72.9; 74.4; 87.3; 104.3; 126.3; 126.6; 127.4; 140.1.

LC-MS: (Method: ASCA-7 MIN-80 deg.M): m/z: [M+H]⁺=398.3, Rₜ 3.5 min.

Only one diastereoisomer could be obtained. Even the ¹H-NMR spectrum of the crude product showed that there must substantially be only one diastereoisomer.

### Alkyne Structural Unit 12:

### 4-((But-2-ynyloxy)methyl)-N,N-dimethyl-1-phenylcyclohexanamine (Ain-12, one of two possible diastereomers)

A solution of 4-but-2-ynyloxymethyl-1-dimethylaminocyclohexane carbonitrile (Nit-03) (4.16 g, 17.8 mmol) in anhydrous tetrahydrofuran (80 ml) was added dropwise with ice cooling to a 2 M solution of phenyl magnesium chloride in tetrahydrofuran (26.6 ml, 53.3 mmol) and the mixture was stirred overnight at room temperature. Saturated ammonium chloride solution and water (15 ml each) were then added dropwise to the mixture with ice cooling. The tetrahydrofuran was distilled off under vacuum and the residue was extracted with diethyl ether (3×40 ml). The combined organic phases were dried with sodium sulfate and concentrated to small volume under vacuum. The crude product (3.53 g) was purified by flash chromatography (370 g, 20×7.6 cm) with ethyl acetate/methanol (9:1). Only one diastereoisomer was isolated.

Yield: 2.47 g (48%), yellowish oil

¹H-NMR (DMSO-d₆): 1.30-1.64 (m, 7H); 1.83 (t, 3H, J=2.3 Hz); 1.92 (s, 6H); 2.58 (br d, 2H, J=12.5 Hz), 3.29 (d, 2H, J=6.3 Hz); 4.06 (q, 2H, J=2.3 Hz); 7.23 (m, 1H); 7.29-7.37 (m, 4H).

### Alkyne Structural Unit 13:

### 4-((4,4-Dimethylpent-2-ynyloxy)methyl)-N,N-dimethyl-1-phenylcyclohexanamine (Ain-13, one of two possible diastereomers)

A solution of 1-(dimethylamino)-4-((4,4-dimethylpent-2-ynyloxy)methyl)cyclohexane carbonitrile (Nit-04) (1.54 g, 5.6 mmol) in absolute tetrahydrofuran (30 ml) was slowly added dropwise under argon to an ice-cooled 2 M solution of phenyl magnesium chloride (1.92 g, 7.0 ml, 14.0 mmol) in tetrahydrofuran and the mixture was then stirred overnight at room temperature. Saturated ammonium chloride solution and water (20 ml each) were then added to the reaction solution, the phases were separated and the aqueous phase was extracted with diethyl ether (3×30 ml). The combined organic phases were washed with saturated sodium chloride solution (30 ml), dried with sodium sulfate and concentrated to small volume under vacuum. 1.61 g of crude product were obtained, which were purified by flash chromatography (100 g, 20×4.0 cm) with dichloromethane/methanol (95:5).

Yield: 942 mg (51%), colourless oil, which slowly crystallises

¹H-NMR (DMSO-d₆): 1.20 (s, 9H); 1.27-1.64 (m, 7H); 1.92 (s, 6H); 2.54-2.59 (m, 2H); 3.29 (d, 2H, J=6.1 Hz); 4.07 (s, 2H); 7.20-7.27 (m, 1H); 7.29-7.37 (m, 4H)

Only one diastereoisomer was isolated. The ¹H-NMR spectrum of the crude product showed that it contained at most 5% of the possible second isomer.

### Iodoaniline Structural Unit 1:

### 2-Iodoaniline (Ian-01)

CAS no.: 615-43-0; at the time of synthesis commercially available from e.g. Aldrich

### Iodoaniline Structural Unit 2:

### 4-Amino-3-iodobenzonitrile (Ian-02))

CAS no.: 33348-34-4; at the time of synthesis commercially available from e.g. Aldrich

### Iodoaniline Structural Unit 3:

### 4-Amino-3-iodobenzotrifluoride (Ian-03)

CAS no.: 163444-17-5; at the time of synthesis commercially available from e.g. ABCR

### Iodoaniline Structural Unit 4:

### 4-Fluoro-2-iodoaniline (Ian-04)

CAS no.: 61272-76-2; at the time of synthesis commercially available from e.g. ABCR

### Iodoaniline Structural Unit 5:

### 2-Amino-3-iodopyridine (Ian-05)

CAS no.: 104830-06-0; at the time of synthesis commercially available from e.g. ABCR

### Iodoaniline Structural Unit 6:

### 4-Amino-3-iodopyridine (Ian-06)

CAS no.: 88511-27-7; at the time of synthesis commercially available from e.g. ABCR

### Iodoaniline Structural Unit 8:

### N-(4-Fluoro-2-iodophenyl)acetamide (Ian-08)

A solution of acetyl chloride (2.55 g, 25.2 mmol) in anhydrous dichloromethane (10 ml) was added at 0° C. to a solution of 4-fluoro-2-iodoaniline (Ian-04) (5.93 g, 25 mmol) and triethylamine (3.5 ml) in anhydrous dichloromethane (10 ml). The mixture was stirred for 1 h at 0° C. and then overnight at room temperature and then made alkaline with 25% potassium carbonate solution. The phases were separated and the aqueous phase was extracted with dichloromethane (2×30 ml). The combined organic phases were dried with sodium sulfate and concentrated to small volume under vacuum. The residue (7.01 g) was taken up in tetrahydrofuran (120 ml) and filtered. The filtrate was concentrated to small volume under vacuum.

Yield: 6.60 g (95%), white solid

Melting point: 142-144° C.

¹H-NMR (DMSO-d₆): 2.03 (s, 3H); 7.25 (dt, 1H, J=8.6, 2.9 Hz); 7.37 (dd, 1H, J=8.8, 5.7 Hz); 7.75 (dd, 1H, J=8.2, 2.9 Hz); 9.46 (s, 1H).

### Iodoaniline Structural Unit 9:

### N-(4-Fluoro-2-iodophenyl)methanesulfonamide (Ian-09)

Methanesulfonyl chloride (1.37 g, 0.94 ml, 12 mmol) was added dropwise at room temperature to a solution of 4-fluoro-2-iodoaniline (Ian-04) (2.38 g, 10 mmol) and 4-(dimethylamino)pyridine (121 mg, 1 mmol) in anhydrous pyridine (25 ml) and then the mixture was refluxed whilst stirring for 12 h. The mixture was cooled to room temperature, diluted with dichloromethane (100 ml) and washed with 5% hydrochloric acid (3×20 ml) and water (3×20 ml). The organic phase was dried with sodium sulfate and concentrated to small volume under vacuum. Toluene was added repeatedly to the residue, which was concentrated to small volume again under vacuum each time. The crude product (2.91 g) was purified by flash chromatography (100 g, 3.7×20 cm) with cyclohexane/ethyl acetate (4:1).

Yield: 2.25 g (71%), beige-coloured solid

Melting point: 90-92° C.

¹H-NMR (DMSO-d₆): 3.03 (s, 3H); 7.29 (m, 1H); 7.41 (dd, 1H, J=5.5, 8.8 Hz); 7.80 (dd, 1H, J=2.9, 8.2 Hz); 9.33 (s, 1H).

### Iodoaniline Structural Unit 11:

### 4-Fluoro-2-iodo-N-methylaniline (Ian-11)

A 1.6 M solution of methyl lithium in tetrahydrofuran (15 ml, 24 mmol) was added at −78° C. to a solution of 4-fluoro-2-iodoaniline (Ian-04) (5.93 g, 25 mmol) in anhydrous tetrahydrofuran (50 ml) and the mixture was stirred at room temperature for 30 min. Dimethyl sulfate (4.75 g, 37.6 mmol) was added to the mixture at −78° C., the mixture was stirred at this temperature for 10 min and then stirred at room temperature for 2 h. Water (15 ml) was then added dropwise to the reaction mixture and the tetrahydrofuran was distilled off under vacuum. The residue was acidified with 1 M hydrochloric acid and extracted with diethyl ether (2×30 ml). The aqueous phase was then made alkaline with 25% potassium carbonate solution and extracted with diethyl ether (3×40 ml). The organic phases from the two extractions were each dried with sodium sulfate and concentrated to small volume under vacuum. The crude product obtained from the basic extract (1.37 g) was purified by flash chromatography (100 g, 4.1×20 cm) with cyclohexane and then cyclohexane/ethyl acetate (95:5).

Yield: 943 mg, yellow oil

This is a mixture of products which also includes the target compound.

The crude product (2.78 g) obtained from the acid extract was likewise purified by flash chromatography (200 g, 5.6×20 cm) with cyclohexane.

Yield: 1.10 g (17%), yellow oil, comprising 88% of the title compound and 12% of the corresponding dimethyl derivative.

¹H-NMR (DMSO-d₆): 2.62 (s, 0.84H); 2.71 (d, 3H, J=4.9 Hz); 4.87 (q, 1H, J=4.9 Hz); 6.48 (dd, 1H, J=5.0, 9.0 Hz); 7.10 (m, 1H); 7.22 (m, 0.24 Hz); 7.51 (dd, 1H, J=2.9, 8.1 Hz); 7.68 (m, 0.12H).

### Iodoaniline Structural Unit 12:

### 4-Trifluoromethoxy-2-iodoaniline (Ian-12)

CAS no.: 845866-79-7; at the time of synthesis commercially available from e.g. ABCR

### Example 1

### [1-Phenyl-4-(2-triethylsilanyl-1H-indol-3-ylmethoxymethyl)cyclohexyl]dimethylamine (one of two possible diastereomers)

A mixture of Ain-01 (560 mg, 1.45 mmol), 2-iodoaniline (Ian-01) (381 mg, 1.74 mmol), [1,3-bis-(2,6-diisopropylphenyl)imidazol-2-ylidene]-(3-chloropyridyl)palladium(II) chloride (PEPPSI, 197 mg, 0.29 mmol) and sodium carbonate (768 mg, 7.25 mmol) in anhydrous N,N-dimethyl formamide (10 ml) was stirred for 18 h at 100° C. The solvent was removed under vacuum and the residue divided between water and diethyl ether (50 ml each). The aqueous phase was extracted with diethyl ether (30 ml), the combined organic phases were washed with water and sodium thiosulfate solution (30 ml each), dried with sodium sulfate and concentrated to small volume under vacuum. The crude product (1.00 g) was purified by flash chromatography (100 g, 20×4.0 cm) with ethyl acetate/cyclohexane (1:3).

Yield: 259 mg (37%), brown oil

¹H-NMR (DMSO-d₆): 0.86-0.99 (m, 15H); 1.27-1.57 (m, 7H); 1.88 (s, 6H); 2.53-2.60 (m, 2H); 3.33-3.36 (m, 2H); 4.62 (s, 2H); 6.99 (ddd, 1H, J=7.9, 1.1, 1.0 Hz); 7.08 (ddd, 1H, J=8.1, 1.2, 1.0 Hz); 7.18-7.25 (m, 1H); 7.28-7.34 (m, 4H); 7.40 (dt, 1H, J=8.1, 0.9 Hz); 7.58 (d, 1H, J=7.3 Hz); 10.67 (s, 1H).

¹³C-NMR (DMSO-d₆): 3.2; 7.3; 24.5; 32.1; 37.1; 37.4; 58.5; 64.5; 74.8; 111.3; 118.5; 121.3; 121.4; 126.1; 126.5; 127.2; 128.5; 133.3; 138.6; 139.4.

### Example 2

### [1-Phenyl-4-(1H-indol-3-ylmethoxymethyl)cyclohexyl]dimethylamine (one of two possible diastereomers)

Activated molecular sieve 4 Å (2.00 g) was added to a suspension of benzyltrimethyl ammonium fluoride monohydrate (152 mg, 0.9 mmol) in anhydrous tetrahydrofuran (20 ml) and the mixture was stirred for 1 h at room temperature. A solution of Example 1 (140 mg, 0.3 mmol) in anhydrous tetrahydrofuran (7 ml) was then added to this suspension and the mixture was refluxed whilst stirring for 1 h. The reaction mixture was then filtered, the filtrate concentrated to small volume under vacuum and the residue purified by flash chromatography (10 g, 20×1.5 cm) with ethyl acetate/methanol (95:5).

Yield: 34 mg (31%), oil

¹H-NMR (DMSO-d₆): 1.48-1.78 (m, 7H); 2.03 (s, 6H); 2.47 (br s, 2H); 3.45 (d, 2H, J=6.7 Hz); 4.74 (s, 2H); 7.10-7.40 (m, 9H); 7.74 (d, 1H, J=7.7 Hz); 8.17 (br s, 1H).

¹³C-NMR (DMSO-d₆): 24.2; 30.1; 34.8; 37.5; 55.1; 65.0; 72.6; 111.2; 113.3; 113.9; 119.3; 119.6; 122.1; 124.2; 126.9; 127.2; 127.7; 128.1; 136.5.

### Example 3

### 1-Phenyl-4-(((2-(tert-butyldimethylsilyl)-5-cyano-1H-indol-3-yl)methoxy)methyl)-N,N-dimethylcyclohexanamine (one of two possible diastereomers)

A mixture of Ain-02 (350 mg, 0.9 mmol), 4-amino-3-iodobenzonitrile (Ian-02) (268 mg, 1.1 mmol), [1,3-bis-(2,6-diisopropylphenyl)imidazol-2-ylidene]-(3-chloropyridyl)palladium(II) chloride (PEPPSI, 122 mg, 0.18 mmol) and sodium carbonate (477 mg, 4.5 mmol) in anhydrous N,N-dimethyl formamide (10 ml) was stirred for 24 h at 100° C. The reaction mixture was then concentrated to small volume under vacuum, toluene added repeatedly to the residue and the residue concentrated to small volume again each time. The residue was divided between water and diethyl ether (10 ml each). The aqueous phase was extracted with diethyl ether (3×10 ml) and the combined organic phases were washed with water and sodium thiosulfate solution (20 ml each). The organic phase was dried with sodium sulfate and concentrated to small volume under vacuum. The crude product (400 mg) was purified by flash chromatography (38 g, 20×2.5 cm) with ethyl acetate/cyclohexane (1:2).

Yield: 147 mg (32%), yellowish solid

Melting point: 72-75° C.

¹H-NMR (DMSO-d₆): 0.41 (s, 6H); 0.89 (s, 9H); 1.28-1.38 (m, 2H); 1.41-1.65 (m, 5H); 1.90 (s, 6H); 2.56 (d, 2H, J=13.3 Hz); 3.38 (d, 2H, J=6.4 Hz); 4.65 (s, 2H); 7.19-7.33 (m, 5H); 7.44 (d, 1H, J=8.4 Hz); 7.56 (d, 1H, J=8.4 Hz); 8.10 (s, 1H); 11.24 (s, 1H).

¹H-NMR (CDCl₃): 0.43 (s, 6H); 0.95 (s, 9H); 150-1.80 (m, 7H); 2.03 (s, 6H); 2.50-2.56 (m, 2H); 3.48 (d, 2H, J=6.6 Hz); 4.72 (s, 2H); 7.22-7.38 (m, 5H); 7.41 (d, 2H, J=1.0 Hz); 8.13 (s, 1H); 8.29 (s, 1H).

¹³C-NMR (CDCl₃): −5.2; 17.4; 24.9; 26.4; 26.9; 32.3; 37.1; 37.8; 59.4; 65.3; 76.0; 102.7; 111.6; 120.9; 123.6; 125.3; 125.6; 126.3; 126.9; 127.3; 128.7; 136.8; 140.0.

### Example 4

### 1-Phenyl-4-(((5-cyano-2-(triethylsilyl)-1H-indol-3-yl)methoxy)methyl)-N,N-dimethylcyclohexanamine (one of two possible diastereomers)

A mixture of Ain-01 (385 mg, 1 mmol), 4-amino-3-iodobenzonitrile (Ian-02) (293 mg, 1.2 mmol), [1,3-bis-(2,6-diisopropylphenyl)imidazol-2-ylidene]-(3-chloropyridyl)palladium(II) chloride (PEPPSI, 136 mg, 0.2 mmol) and sodium carbonate (530 mg, 5 mmol) in anhydrous N,N-dimethyl formamide (10 ml) was stirred for 18 h at 100° C. The solvent was then removed under vacuum and the residue divided between water and diethyl ether (30 ml each). The aqueous phase was extracted with diethyl ether (10 ml), the combined organic phases were washed with water and sodium thiosultate solution (30 ml each), dried with sodium sulfate and concentrated to small volume under vacuum. The crude product (500 mg) was purified by flash chromatography (18 g, 20×2.0 cm) with ethyl acetate/cyclohexane (1:3).

Yield: 328 mg (65%), colourless solid

Melting point: 48-52° C.

¹H-NMR (CDCl₃): 0.89-0.97 (m, 6H); 1.00-1.06 (m, 9H); 1.47-1.80 (m, 7H); 2.02 (s, 6H); 2.49-2.56 (m, 2H); 3.47 (d, 2H, J=6.7 Hz); 4.72 (s, 2H); 7.22-7.38 (m, 5H); 7.41 (d, 2H, J=1.0 Hz); 8.11 (s, 1H); 8.31 (s, 1H).

¹³C-NMR (CDCl₃): 3.7; 7.3; 24.9; 26.9; 32.3; 37.1; 37.8; 59.4; 65.1; 75.8; 102.6; 111.6; 120.9; 123.4; 125.2; 125.4; 126.3; 126.9; 127.3; 128.7; 136.6; 139.5; 140.0.

### Example 5

### 1-Phenyl-4-(((5-cyano-1H-indol-3-yl)methoxy)methyl)-N,N-dimethylcyclohexanamine (one of two possible diastereomers)

Activated molecular sieve 4 Å (2.00 g) was added to a suspension of benzyltrimethyl ammonium fluoride monohydrate (218 mg, 1.29 mmol) in anhydrous tetrahydrofuran (20 ml) and the mixture was stirred for 1 h at room temperature. A solution of Example 4 (218 mg, 0.43 mmol) in anhydrous tetrahydrofuran (10 ml) was then added to this suspension and the mixture was refluxed whilst stirring for 1 h. The reaction mixture was then filtered, the filtrate concentrated to small volume under vacuum and the residue purified by flash chromatography (5 g, 15×0.9 cm) with ethyl acetate/methanol (95:5).

Yield: 98 mg (59%), oil

¹H-NMR (CDCl₃): 1.50-1.67 (m, 6H); 1.70-1.80 (br s, 1H); 2.02 (s, 6H); 2.42-2.51 (m, 2H); 3.45 (d, 2H, J=6.7 Hz); 4.70 (s, 2H); 7.20-7.43 (m, 8H); 8.08 (s, 1H); 9.01 (s, 1H).

¹³C-NMR (CDCl₃): 24.7; 32.0; 36.8; 37.7; 59.6; 64.8; 75.2; 102.7; 112.0; 114.5; 120.8; 124.8; 125.1; 125.2; 125.5; 126.4; 126.9; 127.4; 130.2; 138.2; 139.0.

### Example 6

### 1-Phenyl-4-(((2-(tert-butyldimethylsilyl)-5-trifluoromethyl-1H-indol-3-yl)methoxy)methyl)-N,N-dimethylcyclohexanamine (one of two possible diastereomers)

A mixture of Ain-02 (385 mg, 1 mmol), 4-amino-3-iodobenzotrifluoride (Ian-03) (344 mg, 1.2 mmol), [1,3-bis-(2,6-diisopropylphenyl)imidazol-2-ylidene]-(3-chloropyridyl)palladium(II) chloride (PEPPSI, 136 mg, 0.2 mmol) and sodium carbonate (530 mg, 5 mmol) in anhydrous N,N-dimethyl formamide (10 ml) was stirred for 24 h at 100° C. The solvent was then removed under vacuum and the residue divided between water and diethyl ether (10 ml each). The aqueous phase was extracted with diethyl ether (3×10 ml) and the combined organic phases were washed with water and sodium thiosultate solution (20 ml each). The organic phase was dried with sodium sulfate, concentrated to small volume under vacuum and the crude product was purified by flash chromatography (38 g, 20×2.5 cm) with ethyl acetate/cyclohexane (1:4).

Yield: 326 mg (60%)

Melting point: 48-50° C.

¹H-NMR (DMSO-d₆): 0.41 (s, 6H); 0.90 (s, 9H); 1.28-1.66 (m, 7H); 1.89 (s, 6H); 2.55 (d, 2H, J=13.0 Hz); 3.37 (d, 2H, J=6.2 Hz); 4.68 (s, 2H); 7.18-7.24 (m, 1H); 7.28-7.35 (m, 4H); 7.39 (dd, 1H, J=8.5, 1.3 Hz); 7.58 (d, 1H, J=8.5 Hz); 7.97 (s, 1H); 11.10 (s, 1H).

¹³C-NMR (CDCl₃): −5-2; 17.7; 24.9; 26.4; 32.3; 37.1; 37.8; 59.4; 65.5; 75.7; 110.9; 117.7 (q, J=4.6 Hz); 119.3 (q, J=3.6 Hz); 121.9; 122.2; 123.8; 126.3; 126.9; 127.3; 128.3; 136.0; 119.5, 139.7.

### Example 7

### 1-Phenyl-4-(((5-trifluoromethyl-2-(triethylsilyl)-1H-indol-3-yl)methoxy)methyl)-N,N-dimethylcyclohexanamine (one of two possible diastereomers)

A mixture of Ain-01 (385 mg, 1 mmol), 4-amino-2-iodobenzotrifluoride (Ian-03) (344 mg, 1.2 mmol), [1,3-bis-(2,6-diisopropylphenyl)imidazol-2-ylidene]-(3-chloropyridyl)palladium(II) chloride (PEPPSI, 136 mg, 0.2 mmol) and sodium carbonate (530 mg, 5 mmol) in anhydrous N,N-dimethyl formamide (10 ml) was stirred for 18 h at 100° C. The solvent was removed under vacuum and the residue divided between water and diethyl ether (30 ml each). The aqueous phase was extracted with diethyl ether (10 ml), the combined organic phases were washed with water and sodium thiosulfate solution (30 ml each), dried with sodium sulfate and concentrated to small volume under vacuum. The crude product (600 mg) was purified by flash chromatography (10 g, 20×1.5 cm) with ethyl acetate/cyclohexane (1:9).

Yield: 329 mg (60%), oil

¹H-NMR (DMSO-d₆): 0.85-0.98 (m, 15H); 1.25-1.67 (m, 7H); 1.88 (s, 6H); 2.57 (br s, 2H); 3.34 (br d, 2H, J=6.2 Hz); 4.68 (s, 2H); 7.19-7.41 (m, 6H); 7.58 (d, 1H, J=8.5 Hz); 7.97 (s, 1H); 11.10 (s, 1H).

¹³C-NMR (DMSO-d₆): 3.1; 7.2; 24.5; 26.3; 28.9; 32.0; 37.0; 37.4; 58.5; 64.4; 74.5; 112.0; 116.4; 117.7; 119.2; 119.6; 122.6; 124.3; 126.1; 126.5; 127.2; 127.6; 136.1; 139.3; 140.1.

### Example 8

### 1-Phenyl-4-(((5-trifluoromethyl-1H-indol-3-yl)methoxy)methyl)-N,N-dimethylcyclohexanamine (one of two possible diastereomers)

Activated molecular sieve 4 Å (2.00 g) was added to a suspension of benzyltrimethyl ammonium fluoride monohydrate (237 mg, 1.4 mmol) in anhydrous tetrahydrofuran (20 ml) and the mixture was stirred for 1 h at room temperature. A solution of Example 7 (250 mg, 0.46 mmol) in anhydrous tetrahydrofuran (15 ml) was then added to this suspension and the mixture was refluxed whilst stirring for 1 h. The reaction mixture was then filtered, the filtrate concentrated to small volume under vacuum and the residue purified by flash chromatography (10 g, 20×1.5 cm) with ethyl acetate/methanol (95:5).

Yield: 70 mg (35%), oil

¹H-NMR (CDCl₃): 1.50-1.80 (m, 7H); 2.02 (s, 6H); 2.40-2.50 (m, 2H); 3.47 (d, 2H, J=6.8 Hz); 4.74 (s, 2H); 7.13-7.45 (m, 8H); 8.05 (s, 1H); 8.69 (s, 1H).

¹³C-NMR (CDCl₃): 24.7; 32.0; 36.8; 37.7; 59.7; 64.8; 65.0; 74.9; 111.4; 114.6; 117.3; 117.4; 118.9; 122.1 (q, J=32 Hz); 124.0; 125.0; 126.4; 126.5; 126.7; 127.0; 127.4; 129.7; 137.8; 139.0.

### Example 9

### 1-Phenyl-4-(((5-fluoro-2-(triethylsilyl)-1H-indol-3-yl)methoxy)methyl)-N,N-dimethylcyclohexanamine (one of two possible diastereomers)

A mixture of Ain-01 (485 mg, 1.26 mmol), 4-fluoro-2-iodoaniline (Ian-04) (357 mg, 1.51 mmol), [1,3-bis-(2,6-diisopropylphenyl)imidazol-2-ylidene]-(3-chloropyridyl)palladium(II) chloride (PEPPSI, 171 mg, 0.25 mmol) and sodium carbonate (668 mg, 6.3 mmol) in anhydrous N,N-dimethyl formamide (10 ml) was stirred for 18 h at 100° C. The solvent was removed under vacuum and the residue divided between water and diethyl ether (50 ml each). The aqueous phase was extracted with diethyl ether (30 ml), the combined organic phases were washed with water and sodium thiosulfate solution (30 ml each), dried with sodium sulfate and concentrated to small volume under vacuum. The crude product (600 mg) was purified by flash chromatography (10 g, 20×1.5 cm) with ethyl acetate/cyclohexane (1:9).

Yield: 285 mg (46%), oil

¹H-NMR (DMSO-d₆): 0.84-0.99 (m, 15H); 1.27-1.65 (m, 7H); 1.89 (s, 6H); 2.56 (d, 2H, J=12.6 Hz); 3.30-3.35 (2H, under the HDO signal); 4.59 (s, 2H); 6.93 (dt, 1H, J=9.2, 2.5 Hz); 7.18-7.41 (m, 7H); 10.77 (s, 1H).

¹³C-NMR (DMSO-d₆): 3.1; 7.2; 24.5; 26.3; 28.9; 32.0; 37.0; 37.4; 58.5; 64.4; 74.8; 102.9 (d, J=23 Hz); 109.6 (d, J=26 Hz); 112.2 (d, J=11 Hz); 121.5 (d, J=5 Hz); 126.1; 126.5; 127.2; 128.7 (d, J=10 Hz); 135.3; 135.8; 139.4; 156.7 (d, J=232 Hz).

### Example 10

### 1-Phenyl-4-(((5-fluoro-1H-indol-3-yl)methoxy)methyl)-N,N-dimethylcyclohexanamine (one of two possible diastereomers)

Activated molecular sieve 4 Å (2.00 g) was added to a suspension of benzyltrimethyl ammonium fluoride monohydrate (237 mg, 1.4 mmol) in anhydrous tetrahydrofuran (20 ml) and the mixture was stirred for 1 h at room temperature. A solution of Example 9 (230 mg, 0.46 mmol) in anhydrous tetrahydrofuran (15 ml) was then added to this suspension and the mixture was refluxed whilst stirring for 1 h. The reaction mixture was then filtered, the filtrate concentrated to small volume under vacuum and the residue purified by flash chromatography (10 g, 20×1.5 cm) with ethyl acetate/methanol (95:5).

Yield: 91 mg (66%), oil

¹H-NMR (CDCl₃): 1.42-1.80 (m, 7H); 2.03 (s, 6H); 2.47 (br d, 2H, J=6.9 Hz); 3.44 (d, 2H, J=6.7 Hz); 4.67 (s, 2H); 6.95 (dt, 1H, J=9.0, 2.4 Hz); 7.21-7.40 (m, 8H); 8.27 (s, 1H).

¹³C-NMR (CDCl₃): 24.7; 32.0; 36.9; 37.8; 65.1; 74.9; 104.4 (d, J=23 Hz); 110.6 (d, J=26 Hz); 111.6 (d, J=10 Hz); 114.0; 125.3; 126.5; 127.0; 127.5; 127.7 (d, J=10 Hz); 133.0; 157.9 (d, J=235 Hz).

### Example 11

### 1-Phenyl-N,N-dimethyl-4-(((2-(triethylsilyl)-1H-pyrrolo[2,3-b]pyridin-3-yl)methoxy)methyl)cyclohexanamine (one of two possible diastereomers)

A mixture of Ain-01 (385 mg, 1 mmol), 2-amino-3-iodopyridine (Ian-05) (264 mg, 1.2 mmol), [1,3-bis-(2,6-diisopropylphenyl)imidazol-2-ylidene]-(3-chloropyridyl)palladium(II) chloride (PEPPSI, 136 mg, 0.2 mmol) and sodium carbonate (530 mg, 5 mmol) in anhydrous N,N-dimethyl formamide (10 ml) was stirred for 18 h at 100° C. The solvent was then removed under vacuum and the residue divided between water and diethyl ether (30 ml each). The aqueous phase was extracted with diethyl ether (10 ml), the combined organic phases were washed with water and sodium thiosulfate solution (30 ml each), dried with sodium sulfate and concentrated to small volume under vacuum. The crude product (560 mg) was purified by flash chromatography (18 g, 20×2.0 cm) with ethyl acetate/cyclohexane (1:2).

Yield: 225 mg (47%), oil

¹H-NMR (CDCl₃): 0.88-0.96 (m, 15H); 1.45-1.78 (m, 7H); 2.02 (s, 6H); 2.49-2.56 (m, 2H); 3.45 (d, 2H, J=6.5 Hz); 4.72 (s, 2H); 7.05-7.09 (m, 1H); 7.22-7.37 (m, 5H); 8.03-8.06 (m, 1H); 8.27-8.31 (m, 1H); 8.68 (br s, 1H).

¹³C-NMR (CDCl₃): 3.8; 7.4; 24.9; 26.9; 32.3; 37.2; 37.8; 59.4; 65.6; 75.5; 115.8; 121.0; 121.3; 126.3; 126.9; 127.3; 127.8; 134.3; 139.6; 143.8; 150.6.

### Example 12

### 4-(((1H-Pyrrolo[2,3-b]pyridin-3-yl)methoxy)methyl)-1-phenyl-N,N-dimethylcyclohexanamine (one of two possible diastereomers)

Activated molecular sieve 4 Å (2.00 g) was added to a suspension of benzyltrimethyl ammonium fluoride monohydrate (191 mg, 1.13 mmol) in anhydrous tetrahydrofuran (20 ml) and the mixture was stirred for 1 h at room temperature. A solution of Example 11 (180 mg, 0.37 mmol) in anhydrous tetrahydrofuran (15 ml) was then added to this suspension and the mixture was refluxed whilst stirring for 1 h. The reaction mixture was then filtered, the filtrate concentrated to small volume under vacuum and the residue purified by flash chromatography (5 g, 15×0.9 cm) with ethyl acetate/methanol (9:1).

Yield: 36 mg (27%), oil

¹H-NMR (CDCl₃): 1.45-1.70 (m, 8H); 2.02 (s, 6H); 2.45-2.60 (m, 1H); 3.44 (d, 2H, J=6.6 Hz); 4.72 (s, 2H); 7.12 (dd, 1H, J=7.8, 4.8 Hz); 7.21-7.39 (m, 6H); 8.07 (dd, 1H, J=7.8, 1.5 Hz); 8.34 (dd, 1H, J=4.8, 1.5 Hz); 10.49 (s, 1H).

¹³C-NMR (CDCl₃): 24.8; 32.1; 37.0; 37.8; 45.3; 59.6; 65.3; 75.0; 112.2; 115.5; 115.8; 119.9; 124.0; 126.4; 126.9; 127.4; 127.7; 128.0; 128.2; 129.1; 139.4; 143.0; 143.1; 149.2.

### Example 13

### 1-Phenyl-4-(((2-(tert-butyldimethylsilyl)-1H-pyrrolo[3,2-c]pyridin-3-yl)methoxy)methyl)-N,N-dimethylcyclohexanamine (one of two possible diastereomers)

A mixture of Ain-02 (385 mg, 1 mmol), 4-amino-3-iodopyridine (Ian-06) (264 mg, 1.2 mmol), [1,3-bis-(2,6-diisopropylphenyl)imidazol-2-ylidene]-(3-chloropyridyl)palladium(II) chloride (PEPPSI, 136 mg, 0.2 mmol) and sodium carbonate (530 mg, 5 mmol) in anhydrous N,N-dimethyl formamide (10 ml) was stirred for 24 h at 120° C. The solvent was then removed under vacuum and the residue divided between water and diethyl ether (10 ml each). The aqueous phase was extracted with diethyl ether (3×10 ml) and the combined organic phases were washed with water and sodium thiosulfate solution (20 ml each). The organic phase was dried with sodium sulfate and concentrated to small volume under vacuum. The crude product (600 mg) was purified by flash chromatography (38 g, 20×2.5 cm) with ethyl acetate/methanol (1:4).

Yield: 185 mg (39%), colourless solid

Melting point: 95-100° C.

¹H-NMR (DMSO-d₆): 0.41 (s, 6H); 1.17 (s, 9H); 1.30-1.57 (m, 6H); 1.57-1.66 (br s, 1H); 1.92 (s, 6H); 2.56 (d, 2H, J=12.9 Hz); 3.38 (d, 2H, J=6.3 Hz); 4.68 (s, 2H); 7.20-7.26 (br s, 1H); 7.31-7.38 (m, 5H); 8.15 (d, 1H, J=5.7 Hz); 8.90 (s, 1H), 11.08 (s, 1H).

¹³C-NMR (DMSO-d₆): −5.2; 16.9; 24.4; 26.3; 31.8; 36.8; 37.4; 64.6; 75.0; 106.5; 11.8; 125.3; 126.2; 126.6; 127.2; 135.1; 140.0; 141.7; 142.2.

### Example 14

### 1-Phenyl-N,N-dimethyl-4-(((2-(triethylsilyl)-1H-pyrrolo[3,2-c]pyridin-3-yl)methoxy)methyl)cyclohexanamine (one of two possible diastereomers)

A mixture of Ain-01 (385 mg, 1 mmol), 4-amino-2-iodopyridine (Ian-06) (264 mg, 1.2 mmol), [1,3-bis-(2,6-diisopropylphenyl)imidazol-2-ylidene]-(3-chloropyridyl)palladium(II) chloride (PEPPSI, 136 mg, 0.2 mmol) and sodium carbonate (530 mg, 5 mmol) in anhydrous N,N-dimethyl formamide (10 ml) was stirred for 18 h at 120° C. The solvent was then removed under vacuum and the residue divided between water and diethyl ether (30 ml each). The aqueous phase was extracted with diethyl ether (10 ml), the combined organic phases were washed with water and sodium thiosulfate solution (30 ml each), dried with sodium sulfate and concentrated to small volume under vacuum. The crude product (560 mg) was purified by flash chromatography (10 g, 20×1.5 cm) with ethyl acetate/methanol (4:1).

Yield: 262 mg (55%), oil

¹H-NMR (CDCl₃): 0.87-1.06 (m, 15H); 1.50-1.80 (m, 8H); 2.04 (s, 6H); 2.46-2.57 (m, 2H); 3.48 (d, 2H, J=6.7 Hz); 4.77 (s, 2H); 7.21-7.38 (m, 5H); 8.28 (d, 1H, J=5.8 Hz); 8.46 (s, 1H); 9.07 (s, 1H).

¹³C-NMR (CDCl₃): 3.7; 7.3; 14.2; 24.8; 31.9; 36.8; 37.8; 65.3; 75.4; 106.1; 122.9; 125.8; 126.5; 127.0; 127.4; 135.1; 141.0; 142.1; 143.1.

### Example 15

### 4-(((1H-Pyrrolo[3,2-c]pyridin-3-yl)methoxy)methyl)-1-phenyl-N,N-dimethylcyclohexanamine (one of two possible diastereomers)

Activated molecular sieve 4 Å (1.20 g) was added to a suspension of benzyltrimethyl ammonium fluoride monohydrate (157 mg, 0.93 mmol) in anhydrous tetrahydrofuran (20 ml) and the mixture was stirred for 1 h at room temperature. A solution of Example 14 (150 mg, 0.31 mmol) in anhydrous tetrahydrofuran (15 ml) was then added to this suspension and the mixture was refluxed whilst stirring for 1 h. The reaction mixture was then filtered, the filtrate concentrated to small volume under vacuum and the residue purified by flash chromatography (5 g, 15×0.9 cm) with ethyl acetate/methanol (4:1).

Yield: 78 mg (70%), oil

¹H-NMR (CDCl₃): 1.50-1.68 (m, 6H); 1.73 (br s, 1H); 1.99 (s, 6H); 2.40-2.50 (m, 2H); 3.47 (d, 2H, J=7.0 Hz); 4.75 (s, 2H); 7.17-7.37 (m, 7H); 8.3 (d, 1H, J=5.9 Hz); 9.07 (d, 1H, J=1.2 Hz); 10.0 (br s, 1H).

¹³C-NMR (CDCl₃): 24.7; 32.0; 36.9; 37.7; 59.6; 64.9; 75.1; 106.7; 113.6; 124.1; 124.5; 126.4; 126.9; 127.4; 139.2; 140.5; 140.7; 142.6.

### Example 16

### 1-(3-(((4-(Dimethylamino)-4-phenylcyclohexyl)methoxy)methyl)-5-fluoro-2-(triethylsilyl)-1H-indol-1-yl)ethanone (one of two possible diastereomers)

A mixture of Ain-01 (578 mg, 1.5 mmol), [1,3-bis-(2,6-diisopropylphenyl)imidazol-2-ylidene]-(3-chloropyridyl)palladium(II) chloride (PEPPSI, 204 mg, 0.3 mmol), N-(4-fluoro-2-iodophenyl)acetamide (Ian-8) (521 mg, 1.9 mmol) and sodium carbonate (793 mg, 7.5 mmol) was evacuated for 30 min in an oil pump. Anhydrous N,N-dimethyl formamide (8 ml), which had previously been flushed with argon for 1 h, was then added via a Schlenk attachment. The reaction mixture was then stirred for 18 h at 100° C. and then concentrated to small volume under vacuum. Toluene was repeatedly added to the residue, which was concentrated to small volume under vacuum each time and then divided between water and ethyl acetate (20 ml each). The phases were separated and the aqueous phase was extracted with ethyl acetate (3×30 ml). The combined organic phases were washed with 1 M sodium thiosulfate solution (30 ml) and saturated sodium chloride solution (50 ml). The combined organic phases were dried with sodium sulfate and concentrated to small volume under vacuum. The crude product (1.17 g) was purified by flash chromatography (100 g, 20×3.7 cm) with ethyl acetate/methanol (9:1). The mixture of substances obtained was purified again by flash chromatography (85 g, 20×3.4 cm) with cyclohexane/ethyl acetate (4:1).

Yield: 365 mg (45%), yellowish solid

Melting point: 90-92° C.

¹H-NMR (DMSO-d₆): 0.84-0.99 (m, 15H); 1.20-1.70 (m, 7H); 1.89 (s, 6H); 2.57 (br d, 2H, J=13.0 Hz); 2.84 (s, 3H); 3.37 (d, 2H, J=6.1 Hz); 4.60 (s, 2H); 7.16-7.40 (m, 6H); 7.48 (dd, 1H, J=2.6, 9.1 Hz); 7.83 (dd, 1H, J=4.2, 9.2 Hz).

¹³C-NMR (DMSO-d₆): 5.1; 7.9; 24.4; 26.1; 31.9; 36.9; 37.4; 58.5; 63.4; 75.2; 105.1 (d, J=23 Hz); 112.3 (d, J=25 Hz); 115.6 (d, J=9 Hz); 126.1; 126.5; 127.2; 130.9; 133.1; 133.4 (d, J=9 Hz); 138.4; 139.3; 158.3 (d, J=238 Hz); 170.2.

### Example 17

### 4-(((5-Fluoro-1-(methylsulfonyl)-2-(triethylsilyl)-1H-indol-3-yl)methoxy)methyl)-N,N-dimethyl-1-phenylcyclohexanamine (one of two possible diastereomers)

A mixture of Ain-01 (578 mg, 1.5 mmol), [1,3-bis-(2,6-diisopropylphenyl)imidazol-2-ylidene]-(3-chloropyridyl)palladium(II) chloride (PEPPSI, 204 mg, 0.3 mmol), N-(4-fluoro-2-iodophenyl)methanesulfonamide (Ian-9) (590 mg, 1.9 mmol) and sodium carbonate (793 mg, 7.5 mmol) was evacuated for 30 min in an oil pump. Anhydrous N,N-dimethyl formamide (8 ml), which had previously been flushed with argon for 1 h, was then added via a Schlenk attachment. The reaction mixture was then stirred for 18 h at 100° C. and then concentrated to small volume under vacuum. Toluene was repeatedly added to the residue, which was concentrated to small volume under vacuum each time and then divided between water and ethyl acetate (20 ml each). The phases were separated and the aqueous phase was extracted with ethyl acetate (3×30 ml). The combined organic phases were washed with 1 M sodium thiosulfate solution (30 ml) and saturated sodium chloride solution (50 ml), dried with sodium sulfate and concentrated to small volume under vacuum. The crude product (1.06 g) was purified by flash chromatography (110 g, 20×3.7 cm) with cyclohexane/ethyl:acetate (4:1) and then with cyclohexane/ethyl acetate (2:1).

Yield: 504 mg (58%), brown oil

¹H-NMR (DMSO-d₆): 0.86-1.06 (m, 15H); 1.10-1.70 (m, 7H); 1.89 (s, 6H); 2.56 (br d, 2H, J=12.4 Hz); 3.09 (s, 3H); 3.40 (d, 2H, J=6.1 Hz); 4.61 (s, 2H); 7.20-7.34 (m, 6H); 7.53 (dd, 1H, J=2.5, 9.1 Hz); 7.90 (dd, 1H, J=4.4, 9.1 Hz).

¹³C-NMR (DMSO-d₆): 4.4; 7.5; 24.4; 26.2; 31.9; 36.8; 37.4; 58.5; 63.5; 75.3; 105.5 (d, J=24 Hz); 113.4 (d, J=26 Hz); 115.7 (d, J=10 Hz); 122.5; 126.1; 126.5; 127.1; 132.9 (d, J=10 Hz); 133.6; 135.1; 139.1; 139.3; 159.1 (d, J=239 Hz).

### Example 18

### 1-Phenyl-4-[2-(tert-butyldimethylsilanyl)-1H-indol-3-ylmethoxymethyl]cyclohexyldimethylamine (one of two possible diastereomers)

A mixture of Ain-02 (965 mg, 2.5 mmol), 2-iodoaniline (Ian-01) (655 mg, 3 mmol), palladium(II) acetate (110 mg, 0.5 mmol), tetra-n-butylammonium chloride (655 mg, 2.5 mmol), triphenyl phosphine (260 mg, 1 mmol) and sodium carbonate (1.32 g, 12.5 mmol) in anhydrous N,N-dimethyl formamide (15 ml) was stirred for 18 h at 100° C. The solvent was then removed under vacuum and the residue divided between water and diethyl ether (50 ml each). The aqueous phase was extracted with diethyl ether (30 ml) and the combined organic phases were washed with water and sodium thiosultate solution (30 ml each). The organic phase was dried with sodium sulfate and concentrated to small volume under vacuum. The crude product (247 mg) was purified by flash chromatography (100 g, 20×4.0 cm) with ethyl acetate/cyclohexane (1:2).

Yield: 930 mg (78%), brown oil

¹H-NMR (DMSO-d₆): 0.39 (s, 6H); 0.89 (s, 9H); 1.25-1.60 (m, 7H); 1.89 (s, 6H); 2.56 (d, 2H, J=12.2 Hz); 3.34 (s, 2H); 4.62 (s, 2H); 6.96-7.02 (m, 1H); 7.09 (dt, 1H, J=6.9, 1.0 Hz); 7.17-7.26 (m, 2H); 7.27-7.38 (m, 3H); 7.41 (d, 1H, J=8.1 Hz); 7.59 (d, 1H, J=7.8 Hz); 10.7 (s, 1H).

¹³C-NMR (DMSO-d₆): −5.0; 17.0; 24.5; 26.3; 26.4; 32.1; 37.4; 58.5; 64.8; 75.0; 111.2; 118.5; 118.7; 121.5; 121.6; 126.1; 126.5; 127.2; 128.5; 133.6; 138.6.

### Example 19

### 1-Phenyl-4-(((2-(tert-butyldimethylsilyl)-5-fluoro-1H-indol-3-yl)methoxy)methyl)-N,N-dimethylcyclohexanamine (one of two possible diastereomers)

A mixture of Ain-02 (385 mg, 1 mmol), 4-fluoro-2-iodoaniline (Ian-04) (284 mg, 1.2 mmol), [1,3-bis-(2,6-diisopropylphenyl)imidazol-2-ylidene]-(3-chloropyridyl)palladium(II) chloride (PEPPSI, 136 mg, 0.2 mmol) and sodium carbonate (530 mg, 5 mmol) in anhydrous N,N-dimethyl formamide (10 ml) was stirred for 24 h at 100° C. The solvent was then removed under vacuum and the residue divided between water and diethyl ether (10 ml each). The aqueous phase was extracted with diethyl ether (10 ml) and the combined organic phases were washed with water and sodium thiosulfate solution (20 ml each). The organic phase was dried with sodium sulfate and concentrated to small volume under vacuum. The crude product (500 mg) was purified by flash chromatography (38 g, 20×2.5 cm) with ethyl acetate/cyclohexane (1:2).

Yield: 370 mg (75%), brown oil

¹H-NMR (DMSO-d₆): 0.39 (s, 6H); 0.89 (s, 9H); 1.30-1.60 (m, 7H); 1.92 (s, 6H); 2.57 (d, 2H, J=12.8 Hz); 3.35 (d, 2H, J=6.1 Hz); 4.59 (s, 2H); 6.94 (dt, 1H, J=9.1, 2.5 Hz); 7.19-7.40 (m, 7H); 10.77 (s, 1H).

¹³C-NMR (DMSO-d₆): −5.1; 16.9; 24.5; 26.3; 32.0; 37.1; 37.4; 58.5; 64.7; 75.0; 103.0 (d, 1C, J=23 Hz); 109.7 (d, J=26 Hz); 112.1 (d, J=10 Hz); 121.7 (d, J=5 Hz); 126.1; 126.5; 127.2; 128.7 (d, J=10 Hz); 135.3; 136.1; 139.4; 156.7 (d, J=232 Hz).

### Example 20

### 1-Phenyl-4-(((2-(tert-butyldimethylsilyl)-1H-pyrrolo[2,3-b]pyridin-3-yl)methoxy)methyl)-N,N-dimethylcyclohexanamine (one of two possible diastereomers)

A mixture of Ain-02 (385 mg, 1 mmol), 2-amino-3-iodopyridine (Ian-05) (264 mg, 1.2 mmol), [1,3-bis-(2,6-diisopropylphenyl)imidazol-2-ylidene]-(3-chloropyridyl)palladium(II) chloride (PEPPSI, 136 mg, 0.2 mmol) and sodium carbonate (530 mg, 5 mmol) in anhydrous N,N-dimethyl formamide (10 ml) was stirred for 24 h at 100° C. The solvent was then removed under vacuum and the residue divided between water and diethyl ether (10 ml each). The aqueous phase was extracted with diethyl ether (3×10 ml) and the combined organic phases were washed with water and sodium thiosulfate solution (20 ml each). The organic phase was dried with sodium sulfate and concentrated to small volume under vacuum. The crude product (535 mg) was purified by flash chromatography (38 g, 20×2.5 cm) with ethyl acetate/cyclohexane (1:2), ethyl acetate and ethyl acetate/methanol (9:1).

Yield: 190 mg (40%), brownish solid

Melting point: 73-78° C.

1H-NMR (DMSO-d6): 0.41 (s, 6H); 0.89 (s, 9H); 1.28-1.63 (m, 7H); 1.90 (s, 6H); 2.56 (d, 2H, J=12.6 Hz); 3.35 (d, 2H, J=6.2 Hz); 4.62 (s, 2H); 7.05 (dd, 1H, J=7.8, 4.6 Hz); 7.19-7.24 (m, 1H); 7.28-7.35 (m, 4H); 7.99 (dd, 1H, J=7.8, 1.0 Hz); 8.24 (dd, 1H, J=4.6, 1.5 Hz); 11.31 (s, 1H).

13C-NMR (CDCl3): −5.1; 17.4; 24.8; 26.4; 32.2; 37.1; 37.8; 65.8; 75.5; 115.8; 116.2; 121.1; 121.4; 126.9; 127.4; 127.9; 134.5; 143.9; 150.6.

### Example 21

### 4-(((5-Fluoro-3-methyl-1H-indol-2-yl)methoxy)methyl)-N,N-dimethyl-1-phenylcyclohexanamine (one of two possible diastereomers)

A mixture of Ain-12 (570 mg, 2 mmol), 4-fluoro-2-iodoaniline (Ian-04) (592 mg, 2.5 mmol), palladium(II) acetate (90 mg, 0.38 mmol), tetra-n-butylammonium chloride (564 mg, 2 mmol), triphenyl phosphine (203 mg, 0.80 mmol) and sodium carbonate (1.05 g, 10 mmol) was evacuated for 30 min in an oil pump. Anhydrous N,N-dimethyl formamide (15 ml), which had previously been flushed with argon for 1 h, was then added via a Schlenk attachment. The mixture was then stirred for 18 h at 100° C. The reaction mixture was then concentrated to small volume under vacuum. Toluene was repeatedly added to the residue, which was concentrated to small volume under vacuum each time and then divided between water and ethyl acetate (20 ml each). The phases were separated and the aqueous phase was extracted with ethyl acetate (3×30 ml). The combined organic phases were washed with 1 M sodium thiosultate solution (30 ml) and saturated sodium chloride solution (50 ml). The combined organic phases were dried with sodium sulfate and concentrated to small volume under vacuum. The crude product (1.06 g) was purified by MPLC [230 g, 46×3.6 cm, LiChroprep Si60 (15-25 μm)] with ethyl acetate/methanol (9:1).

Yield: 319 mg (40%), brown oil

¹H-NMR (DMSO-d₆): 1.25-1.70 (m, 7H); 1.90 (s, 6H); 2.21 (s, 3H); 2.56 (br d, 2H, J=11.9 Hz); 3.30 (d, 2H, J=6.3 Hz); 4.55 (s, 2H); 6.88 (ddd, 1H, J=9.5, 8.9, 2.6 Hz); 7.16-7.36 (m, 3H); 7.51-7.68 (m, 4H); 10.93 (s, 1H).

¹³C-NMR (DMSO-d₆): 8.2; 24.4; 32.0; 36.9; 37.4; 58.5; 63.3; 74.8; 102.8 (d, J=23 Hz); 108.1; 109.0 (d, J=26 Hz); 111.8 (d, J=10 Hz); 126.1; 126.5; 127.2; 128.6 (d, J=12 Hz); 131.4; 131.9; 132.1; 132.2; 133.2; 133.9; 139.3; 156.5 (d, J=231 Hz).

### Example 22

### {1-Benzyl-4-[2-(tert-butyldimethylsilanyl)-1H-indol-3-ylmethoxymethyl]cyclohexyl}dimethylamine (non-polar diastereomer)

In a heated flask with a Schlenk attachment a mixture of Ain-04 (400 mg, 1 mmol), 2-iodoaniline (Ian-01) (263 mg, 1.2 mmol), sodium carbonate (530 mg, 5 mmol) and [1,3-bis-(2,6-diisopropylphenyl)imidazol-2-ylidene]-(3-chloropyridyl)palladium(II) dichloride (PEPPSI, 136 mg, 0.2 mmol) was evacuated, flushed with argon and then N,N-dimethyl formamide (6 ml) was added. The reaction mixture was stirred for 20 h at 100° C. and then concentrated to small volume under vacuum. Toluene was added twice to the residue, which was concentrated to dryness again each time, and then diethyl ether (30 ml) was added. The mixture was washed with 1 M sodium thiosultate solution (10 ml) and water (10 ml). The precipitated deposit was filtered off. The organic phase was washed with sodium chloride solution (10 ml), dried with sodium sulfate and concentrated to small volume under vacuum. The crude product (540 mg) was purified by flash chromatography (30 g, 22×2.5 cm) with chloroform/methanol (9:1) and then purified again by flash chromatography (39 g, 22×2.5 cm) with methanol.

Yield: 240 mg (49%), yellowish solid

Melting point: 53-56° C.

¹H-NMR (DMSO-d₆): 0.35 (s, 6H); 0.86 (s, 9H); 0.88-0.99 (m, 1H); 1.09-1.40 (m, 6H); 1.77 (d, J=13.5 Hz, 2H); 2.20 (s, 6H); 2.55 (s, 2H); 3.17-3.22 (m, 2H); 4.54 (s, 2H); 6.95 (t, J=7.4 Hz, 1H); 7.02-7.29 (m, 6H); 7.39 (d, J=8.1 Hz, 1H); 7.53 (d, J=7.9 Hz, 1H); 10.64 (s, 1H).

¹³C-NMR (DMSO-d₆): −5.1; 17.0; 23.9; 26.4; 31.5; 36.2; 36.5; 37.5; 57.1; 64.7; 75.3; 111.2; 118.5; 118.7; 121.5; 125.4; 127.6; 128.4; 130.5; 133.6; 138.6; 139.1.

### Example 23

### [1-Benzyl-4-(2-triethylsilanyl-1H-indol-3-ylmethoxymethyl)cyclohexyl] dimethylamine (non-polar diastereomer)

In a flask with a Schlenk attachment a mixture of 1-benzyl-N,N-dimethyl-4-((3-(triethylsilyl)prop-2-ynyloxy)methyl)cyclohexanamine (Ain-05, non-polar diastereomer) (520 mg, 1.3 mmol), 2-iodoaniline (342 mg, 1.56 mmol), sodium carbonate (689 mg, 6.5 mmol) and [1,3-bis-(2,6-diisopropylphenyl)imidazol-2-ylidene]-(3-chloropyridyl)palladium(II) dichloride (PEPPSI, 133 mg, 0.2 mmol) was evacuated, flushed with argon and then N,N-dimethyl formamide (7 ml) was added. The reaction mixture was stirred for 20 h at 120° C. and then concentrated to small volume under vacuum. Toluene was added twice to the residue, which was concentrated to dryness again each time, and then ethyl acetate (30 ml) was added. The mixture was washed with 1 M sodium thiosultate solution (10 ml). The deposit was filtered off. The organic phase was washed with sodium chloride solution and water (10 ml each), dried with sodium sulfate and concentrated to small volume under vacuum. The crude product (617 mg) was purified by flash chromatography (40 g, 17×3.6 cm) with ethyl acetate/methanol (9:1→4:1).

Yield: 239 mg (38%), brown oil

¹H-NMR (DMSO-d₆): 0.80-0.95 (m, 17H); 1.13-1.37 (m, 5H); 1.77 (d, J=13.1 Hz, 2H); 2.19 (s, 6H); 2.55 (s, 2H); 3.18 (d, J=5.0 Hz, 2H); 4.55 (s, 2H); 6.92-7.26 (m, 7H); 7.38 (d, J=8.1 Hz, 1H); 7.53 (d, J=7.9 Hz, 1H); 10.62 (s, 1H).

¹³C-NMR (DMSO-d₆): 3.1; 7.2; 23.9; 31.5; 36.2; 36.5; 37.4; 57.1; 64.4; 75.1; 11.3; 118.5; 121.3; 121.4; 125.4; 127.6; 128.5; 130.6; 133.3; 138.6; 139.1.

### Example 24

### [1-Benzyl-4-(1H-indol-3-ylmethoxymethyl)cyclohexyl]dimethylamine (non-polar diastereomer)

A mixture of benzyltrimethyl ammonium fluoride monohydrate (202 mg, 1.08 mmol) and activated molecular sieve 4 Å (700 mg) in anhydrous tetrahydrofuran (5 ml) was stirred for 1 h at room temperature. A solution of Example 23 (175 mg, 0.36 mmol) in anhydrous tetrahydrofuran (3 ml) was then added to the mixture and the mixture was refluxed for 2.5 h. The reaction mixture was filtered, the filter residue washed with tetrahydrofuran, the filtrate concentrated to small volume under vacuum and the residue (172 mg) purified by flash chromatography (40 g, 17×3.6 cm) with ethyl acetate/methanol (4:1).

Yield: 82 mg (61%), brown oil

¹H-NMR (DMSO-d₆): 0.86-1.03 (m, 2H); 1.11-1.38 (m, 5H); 1.77 (d, J=12.8 Hz, 2H); 2.20 (s, 6H); 2.55 (s, 2H); 3.15 (s, 2H); 4.53 (s, 2H); 6.97 (ddd, J=8.0, 7.0 and 1.1 Hz, 1H); 7.34 (td, J=8.1, 0.9 Hz, 1H); 7.02-7.29 (m, 7H); 7.50-7.55 (m, 1H); 10.95 (s, 1H).

¹³C-NMR (DMSO-d₆): 23.8; 31.5; 36.2; 36.5; 37.2; 48.5; 57.1; 64.3; 74.7; 111.3; 112.0; 118.5; 118.7; 121.0; 124.5, 125.4; 127.0; 127.6 (2C); 130.6 (2C); 136.3; 139.1.

### Example 25

### 1-Benzyl-4-(((5-fluoro-2-(triethylsilyl)-1H-indol-3-yl)methoxy)methyl)-N,N-dimethylcyclohexanamine (non-polar diastereomer)

In a flask with a Schlenk attachment a mixture of Ain-05 (486 mg, 1.2 mmol), 4-fluoro-2-iodoaniline (Ian-04, 284 mg, 1.2 mmol), sodium carbonate (636 mg, 6.0 mmol) and [1,3-bis-(2,6-diisopropylphenyl)imidazol-2-ylidene]-(3-chloropyridyl)palladium(II) dichloride (PEPPSI, 122 mg, 0.18 mmol) was evacuated, flushed with argon and then N,N-dimethyl formamide (7 ml) was added. The reaction mixture was stirred for 20 h at 100° C. and then concentrated to small volume under vacuum. Toluene was added to the residue, which was concentrated to dryness again and then diethyl ether (30 ml) was added. The suspension was washed with 1 M sodium thiosulfate solution, sodium chloride solution and water (10 ml each), dried with sodium sulfate and concentrated to small volume under vacuum. The crude product (660 mg) was purified by flash chromatography (50 g, 12×4 cm) with methanol and then purified again by flash chromatography (80 g, 12×4 cm) with chloroform/methanol (9:1).

Yield: 388 mg (63%), beige-coloured solid

Melting point: not determinable

¹H-NMR (DMSO-d₆): 0.77-1.00 (m, 15H); 1.12-1.39 (m, 7H); 1.77 (d, J=12.7 Hz, 2H); 2.19 (s, 6H); 2.54 (s, 2H); 3.17 (d, J=5.2 Hz, 2H); 4.51 (s, 2H); 6.91 (dt, J=9.1 and 2.5 Hz, 1H); 7.06-7.40 (m, 7H); 10.75 (s, 1H). Still contains signals for approximately 15% of an impurity, which is the corresponding monomethyl amino derivative or the hydrochloride of the monomethyl or dimethyl amino derivative.

¹³C-NMR (DMSO-d₆): 3.1; 7.2; 22.2; 23.8; 29.1; 31.5; 36.2; 36.5; 37.3; 57.1; 64.3; 73.6; 75.0; 102.8 (d, J=22 Hz); 109.6 (d, J=27 Hz); 112.2 (d, J=10 Hz); 121.4; 125.4; 127.6; 128.2; 128.6; 128.7; 130.5; 135.3; 135.8; 139.1; 156.7 (d, J=231 Hz).

### Example 26

### 1-Benzyl-4-(((2-(tert-butyldimethylsilyl)-5-fluoro-1H-indol-3-yl)methoxy)methyl)-N,N-dimethylcyclohexanamine (non-polar diastereomer)

In a heated flask with a Schlenk attachment a mixture of Ain-04 (400 mg, 1 mmol), 4-fluoro-2-iodoaniline (Ian-04, 284 mg, 1.2 mmol), sodium carbonate (530 mg, 5 mmol) and [1,3-bis-(2,6-diisopropylphenyl)imidazol-2-ylidene]-(3-chloropyridyl)palladium(II) dichloride (PEPPSI, 136 mg, 0.2 mmol) was evacuated, flushed with argon and then N,N-dimethyl formamide (6 ml) was added. The reaction mixture was stirred for 20 h at 100° C. and then concentrated to small volume under vacuum. Toluene was added three times to the residue, which was concentrated to dryness again each time, and then diethyl ether (30 ml) was added. The mixture was washed with water (10 ml) and the precipitated deposit was filtered off. The organic phase was washed with 1 M sodium thiosulfate solution (10 ml), sodium chloride solution (10 ml) and with water (10 ml), dried with sodium sulfate and concentrated to small volume under vacuum. The crude product (590 mg) was purified by flash chromatography (40 g, 23×2.5 cm) with methanol, purified again by flash chromatography (18 g, 18×2 cm) with chloroform/methanol (95:5) and by a further flash chromatography (20 g, 13×2.5 cm) with ethyl acetate/cyclohexane (1:2).

Yield: 289 mg (57%), yellowish solid

Melting point: 58-62° C.

¹H-NMR (DMSO-d₆): 0.35 (s, 6H); 0.85 (s, 9H); 0.87-1.00 (m, 2H); 1.13-1.35 (m, 5H); 1.77 (d, J=13.1 Hz, 2H); 2.20 (s, 6H); 2.55 (s, 2H); 3.19 (d, J=5.4 Hz, 2H); 4.51 (s, 2H); 6.92 (dt, J=9.1 Hz, 1H); 7.07-7.27 (m, 6H); 7.36 (dd, J=8.8 Hz, 1H); 10.74 (s, 1H).

¹³C-NMR (DMSO-d₆): −5.2; 16.9; 23.9; 26.3; 31.5; 36.2; 36.5; 37.4; 57.1; 64.6; 75.2; 103.0 (d, J=23 Hz); 109.7 (d, J=27 Hz); 112.1 (d, J=11 Hz); 121.6 (d, J=5 Hz); 125.4; 127.6; 128.6; 130.5; 135.3; 136.0; 139.1; 156.7 (d, J=232 Hz).

### Example 27

### 1-Benzyl-4-(((5-fluoro-1H-indol-3-yl)methoxy)methyl)-N,N-dimethylcyclohexanamine (non-polar diastereomer)

A mixture of benzyltrimethyl ammonium fluoride monohydrate (292 mg, 1.56 mmol) and activated molecular sieve 4 Å (approx. 1 g) in anhydrous tetrahydrofuran (7 ml) was stirred for 1 h at room temperature. A solution of Example 25 (263 mg, 0.52 mmol) in anhydrous tetrahydrofuran (3 ml) was then added to the mixture and the mixture was refluxed for 2.5 h. The reaction mixture was filtered, the filter residue washed with tetrahydrofuran, the filtrate concentrated to small volume under vacuum and the residue (257 mg) purified by flash chromatography (20 g, 16×2.5 cm) with ethyl acetate/methanol (4:1).

Yield: 112 mg (54%), beige-coloured solid

Melting point: 42-45° C.

¹H-NMR (DMSO-d₆): 0.87-1.02 (m, 2H); 1.12-1.38 (m, 5H); 1.77 (d, J=13.1 Hz, 2H); 2.20 (s, 6H); 2.55 (s, 2H); 3.14 (d, J=4.2 Hz, 2H); 4.50 (s, 2H); 6.86-6.96 (m, 2H); 7.08-7.37 (m, 7H); 11.06 (s, 1H).

¹³C-NMR (DMSO-d₆): 23.8; 31.5; 36.2; 36.5; 37.2; 57.1; 64.1; 74.7; 103.2 (d, J=24 Hz); 109.1 (d, J=27 Hz); 112.3 (d, J=11 Hz); 125.4; 126.5; 127.3; 127.6; 130.6; 132.9; 139.1; 156.7 (d, J=231 Hz).

### Example 28

### 1-Benzyl-N,N-dimethyl-4-(((2-(triethylsilyl)-1H-pyrrolo[2,3-b]pyridin-3-yl)methoxy)methyl)cyclohexanamine (non-polar diastereomer)

In a flask with a Schlenk attachment a mixture of Ain-05 (570 mg, 1.43 mmol), 2-amino-3-iodopyridine (346 mg, 1.57 mmol), sodium carbonate (758 mg, 7.15 mmol) and [1,3-bis-(2,6-diisopropylphenyl)imidazol-2-ylidene]-(3-chloropyridyl)palladium(II) dichloride (PEPPSI, 146 mg, 0.21 mmol) was evacuated, flushed with argon and then N,N-dimethyl formamide (8 ml) was added. The reaction mixture was stirred for 20 h at 110° C. and then concentrated to small volume under vacuum. Toluene was added to the residue, which was concentrated to dryness again and then diethyl ether (40 ml) was added. The solution was washed with 1 M sodium thiosulfate solution, sodium chloride solution and water (20 ml each), dried with sodium sulfate and concentrated to small volume under vacuum. The crude product (508 mg) was purified by flash chromatography (25 g, 20×2.2 cm) with chloroform/methanol (95:5).

Yield: 374 mg (53%), cream-coloured solid

Melting point: 124-127° C.

¹H-NMR (DMSO-d₆): 0.82-1.00 (m, 17H); 1.15-1.35 (m, 5H); 1.76 (d, J=13.2 Hz, 2H); 2.19 (s, 6H); 2.54 (s, 2H); 3.17 (d, J=4.5 Hz, 2H); 4.54 (s, 2H); 7.01 (dd, J=7.85 and 4.6 Hz, 1H); 7.07-7.27 (m, 5H); 7.93 (dd, J=7.9 and 1.4 Hz, 1H); 8.21 (dd, J=4.6 and 1.6 Hz, 1H); 11.27 (s, 1H).

¹³C-NMR (DMSO-d₆): 2.9 (3C); 7.1 (3C); 23.8 (2C); 31.4 (2C), 36.2; 36.5 (2C); 37.3; 57.1; 64.2; 75.0; 115.0; 120.4; 125.4; 126.7; 127.6 (2C); 130.6 (2C); 134.4; 139.1; 143.0; 150.7.

### Example 29

### 1-Benzyl-4-(((2-(tert-butyldimethylsilyl)-1H-pyrrolo[2,3-b]pyridin-3-yl)methoxy)methyl)-N,N-dimethylcyclohexanamine (non-polar diastereomer)

In a heated flask with a Schlenk attachment a mixture of Ain-04 (175 mg, 0.44 mmol), 2-amino-3-iodopyridine (116 mg, 0.53 mmol), sodium carbonate (232 mg, 2.2 mmol) and [1,3-bis-(2,6-diisopropylphenyl)imidazol-2-ylidene]-(3-chloropyridyl)palladium(II) dichloride (PEPPSI, 45 mg, 0.066 mmol) was evacuated, flushed with argon and then N,N-dimethyl formamide (4 ml) was added. The reaction mixture was stirred for 20 h at 100° C. and then concentrated to small volume under vacuum. Toluene was added to the residue, which was concentrated to dryness again and then diethyl ether (30 ml) was added. The mixture was washed with 1 M sodium thiosultate solution (10 ml), the precipitated deposit was filtered off, and the organic phase was washed with sodium chloride solution and with water (10 ml each), dried with sodium sulfate and concentrated to small volume under vacuum. The crude product (430 mg) was purified by flash chromatography (90 g, 20×4 cm) with methanol and then purified again by flash chromatography (30 g, 18×2.5 cm) with chloroform/methanol (9:1).

Yield: 202 mg (93%), yellowish solid

Melting point: 51-56° C.

¹H-NMR (DMSO-d₆): 0.37 (s, 6H); 0.85 (s, 9H); 0.90-1.02 (m, 2H); 1.11-1.43 (m, 5H); 1.76 (br d, J=13.4 Hz, 2H); 2.19 (s, 6H); 2.54 (s, 2H); 3.20 (m, 2H); 4.54 (s, 2H); 7.01 (dd, J=4.63 and 7.85 Hz, 1H); 7.06-7.28 (m, 5H); 7.92 (dd, J=1.50 and 7.85 Hz, 1H); 8.21 (dd, J=1.59 and 4.63 Hz, 1H); 11.30 (s, 1H). The signals caused by an impurity are at 0.05; 0.87; 2.20 and 4.06 ppm.

¹³C-NMR (DMSO-d₆): −5.0; 17.0; 23.7; 25.7; 26.4; 29.1; 31.5; 36.2; 36.5; 37.4; 57.7; 58.0; 64.6; 75.0; 75.3; 1145.0; 120.3; 125.5; 126.9; 127.6; 130.5; 134.5; 139.1; 143.1; 150.7.

### Example 30

### 4-(((1H-Pyrrolo[2,3-b]pyridin-3-yl)methoxy)methyl)-1-benzyl-N,N-dimethylcyclohexanamine (non-polar diastereomer)

A mixture of benzyltrimethyl ammonium fluoride monohydrate (236 mg, 1.26 mmol) and activated molecular sieve 4 Å (approx. 1 g) in anhydrous tetrahydrofuran (7 ml) was stirred for 1 h at room temperature. A solution of Example 28 (205 mg, 0.42 mmol) in anhydrous tetrahydrofuran (3 ml) was then added to the mixture and the mixture was refluxed for 2 h. The reaction mixture was filtered, the filter residue washed with tetrahydrofuran, the filtrate concentrated to small volume under vacuum and the residue (229 mg) purified by flash chromatography (20 g, 21×2.0 cm) with ethyl acetate/methanol (9:1).

Yield: 70 mg (45%), white solid

Melting point: 39-43° C.

¹H-NMR (DMSO-d₆): 0.86-1.01 (m, 2H); 1.14-1.38 (m, 5H); 1.77 (d, J=13.1 Hz, 2H); 2.20 (s, 6H); 2.55 (s, 2H); 3.15 (d, J=4.6 Hz, 2H); 4.52 (s, 2H); 7.03 (dd, J=7.8 and 4.6 Hz, 1H); 7.08-7.26 (m, 5H); 7.39 (d, J=2.4 Hz, 1H); 7.91 (dd, J=7.8 and 1.3 Hz, 1H); 8.19 (dd, J=4.6 and 1.5 Hz, 1H); 11.47 (s, 1H).

¹³C-NMR (DMSO-d₆): 23.8 (2C); 31.4 (2C); 36.2; 36.5 (2C); 37.1; 57.1; 64.3; 74.7; 110.9; 115.2; 119.1; 124.8; 125.4; 126.9; 127.6 (2C); 130.5 (2C); 139.1, 142.6; 148.7.

### Example 31

### 1-Benzyl-N,N-dimethyl-4-(((2-(triethylsilyl)-1H-pyrrolo[3,2-c]pyridin-3-yl)methoxy)methyl)cyclohexanamine (non-polar diastereomer)

In a flask with a Schlenk attachment a mixture of Ain-05 (430 mg, 1.08 mmol), 4-amino-3-iodopyridine (285 mg, 1.3 mmol), sodium carbonate (572 mg, 5.4 mmol) and [1,3-bis-(2,6-diisopropylphenyl)imidazol-2-ylidene]-(3-chloropyridyl)palladium(II) dichloride (PEPPSI, 110 mg, 0.16 mmol) was evacuated, flushed with argon and then N,N-dimethyl formamide (7 ml) was added. The reaction mixture was stirred for 20 h at 120° C. and then concentrated to small volume under vacuum. Toluene was added to the residue, which was concentrated to dryness again and then diethyl ether (15 ml) and ethyl acetate (15 ml) were added. The solution was washed with 1 M sodium thiosultate solution, sodium chloride solution (20 ml) and water (20 ml), dried with sodium sulfate and concentrated to small volume under vacuum. The crude product (581 mg) was purified by flash chromatography (40 g, 23×2.2 cm) with chloroform/methanol (9:1).

Yield: 310 mg (58%), brown oil

¹H-NMR (DMSO-d₆): 0.83-0.96 (m, 15H); 1.13-1.40 (m, 7H); 1.80 (d, J=13.3 Hz, 2H); 2.25 (s, 6H); 2.58 (s, 2H); 3.21 (d, J=4.0 Hz, 2H); 4.61 (s, 2H); 7.09-7.28 (m, 5H); 7.34-7.37 (m, 1H); 8.13 (d, J=5.7 Hz, 1H); 8.84 (s, 1H); 11.08 (s, 1H).

¹³C-NMR (DMSO-d₆): 3.0 (3C); 7.2 (3C); 23.7 (2C); 31.2 (2C), 36.2; 36.5 (2C); 37.2; 64.1; 75.1; 106.6; 121.7; 125.3; 125.6; 127.6 (2C); 130.6 (2C); 135.0; 138.7; 139.7; 141.8.

### Example 32

### 1-Benzyl-4-(((2-(tert-butyldimethylsilyl)-1H-pyrrolo[3,2-c]pyridin-3-yl)methoxy)methyl)-N,N-dimethylcyclohexanamine (non-polar diastereomer)

In a flask with a Schlenk attachment a mixture of Ain-04 (non-polar diastereoisomer) 400 mg, 1 mmol), 4-amino-3-iodopyridine (100 mg, 0.4 mmol), sodium carbonate (530 mg, 5 mmol) and [1,3-bis-(2,6-diisopropylphenyl)imidazol-2-ylidene]-(3-chloropyridyl)palladium(II) chloride (PEPPSI, 68 mg, 0.1 mmol) was evacuated, flushed with argon and then N,N-dimethyl formamide (6 ml) was added. The reaction mixture was stirred for 20 h at 120° C. and then concentrated to small volume under vacuum. Toluene was added twice to the residue, which was concentrated to dryness again each time, and then diethyl ether (20 ml) was added. The mixture was washed with 1 M sodium thiosultate solution (10 ml) and the precipitated deposit was filtered off. The organic phase was washed with sodium chloride solution and water (10 ml each), dried with sodium sulfate and concentrated to small volume under vacuum. The crude product (520 mg) was purified by flash chromatography (30 g, 20×2.5 cm) with chloroform/methanol (9:10:1).

Yield: 179 mg (80%), yellowish solid

Melting point: 139-144° C.

¹H-NMR (DMSO-d₆): 0.37 (s, 6H); 0.85 (s, 9H); 0.93-1.13 (m, 2H); 1.15-1.48 (m, 5H); 1.81-1.94 (m, 2H), 2.37 (br s, 6H), 2.67 (s, 2H), 3.25 (d, J=4.8 Hz, 2H), 4.61 (s, 2H), 7.12-7.31 (m, 5H), 7.41 (dd, J=5.8, 1.0 Hz, 1H), 8.15 (d, J=5.8 Hz, 1H), 8.87 (s, 1H), 11.28 (s, 1H).

¹³C-NMR (DMSO-d₆): −5.1; 16.9; 23.3; 26.3 (3C); 30.7; 36.5; 36.7; 38.9; 64.4; 75.0; 106.9; 122.2; 125.1; 125.9; 127.7 (2C); 130.7 (2C); 136.0; 138.7; 141.2; 142.0.

### Example 33

### 4-(((1H-Pyrrolo[3,2-c]pyridin-3-yl)methoxy)methyl)-1-benzyl-N,N-dimethylcyclohexanamine (non-polar diastereomer)

A mixture of benzyltrimethyl ammonium fluoride monohydrate (275 mg, 1.47 mmol) and activated molecular sieve 4 Å (approx. 1 g) in anhydrous tetrahydrofuran (8 ml) was stirred for 1 h at room temperature. A solution of Example 31 (240 mg, 0.49 mmol) in anhydrous tetrahydrofuran (3 ml) was then added to the mixture and the mixture was refluxed for 2 h. The reaction mixture was filtered, the filter residue washed with tetrahydrofuran, the filtrate concentrated to small volume under vacuum and the residue (200 mg) purified by flash chromatography (10 g, 19×1.8 cm) with methanol.

Yield: 25 mg (14%)

Melting point: not determinable

¹H-NMR (CDCl₃): 1.03-1.19 (m, 2H); 1.25-1.50 (m, 5H); 1.79 (d, J=12.7 Hz, 2H); 2.29 (s, 6H); 2.60 (s, 2H); 3.29-3.32 (m, 2H); 4.65 (s, 2H); 7.06-7.28 (m, 8H); 8.25 (d, J=5.8 Hz, 1H); 8.97 (s, 1H).

¹³C-NMR (CDCl₃): 24.2; 31.8; 37.0; 37.5; 58.4; 64.7; 76.0; 106.7; 113.9; 124.0; 124.4; 125.7; 127.7; 130.8; 139.1; 140.4; 140.5; 142.4.

### Example 34

### 1-Benzyl-N,N-dimethyl-4-(((2-(triethylsilyl)-1H-indol-3-yl)methoxy)methyl)cyclohexanamine (polar diastereomer)

In a flask with a Schlenk attachment a mixture of Ain-06 (650 mg, 1.6 mmol), 2-iodoaniline (385 mg, 1.76 mmol), sodium carbonate (848 mg, 8.0 mmol) and [1,3-bis-(2,6-diisopropylphenyl)imidazol-2-ylidene]-(3-chloropyridyl)palladium(II) dichloride (PEPPSI, 163 mg, 0.24 mmol) was evacuated, flushed with argon and then N,N-dimethyl formamide (10 ml) was added. The reaction mixture was stirred for 18 h at 100° C. and then concentrated to small volume under vacuum. Toluene was added to the residue, which was concentrated to dryness again and then diethyl ether (40 ml) was added. The solution was washed with 1 M sodium thiosultate solution, then with sodium chloride solution and water (20 ml each), dried with sodium sulfate and concentrated to small volume under vacuum. The crude product (960 mg) was purified by flash chromatography (90 g, 20×4 cm) with chloroform/methanol (95:5).

Yield: 380 mg (48%), brown solid

Melting point: not determinable

¹H-NMR (DMSO-d₆): 0.77-1.00 (m, 15H); 1.21-1.77 (m, 9H); 2.40 (br s, 6H); 3.23 (d, J=5.9 Hz, 2H); 4.56 (s, 2H); 6.97 (ddd, J=7.9; 7.9 and 1.0 Hz, 1H); 7.08 (ddd, J=8.1; 8.1 and 1.2 Hz, 1H); 7.13-7.36 (m, 5H); 7.40 (d, J=8.1 Hz, 1H); 7.53 (d, J=7.8 Hz, 1H); 10.7 (s, 1H). A methylene group is overlaid by the HDO signal.

¹³C-NMR (DMSO-d₆): 3.1; 7.2; 24.3 (br); 25.8; 29.8; 34.8 (br); 36.1; 36.3; 37.2; 64.5; 72.6 (br); 73.5; 11.3; 118.4; 118.5; 121.23; 121.34; 127.0 (br); 128.4; 130.4; 130.5; 133.3; 138.6.

### Example 35

### 1-Benzyl-4-(((2-(tert-butyldimethylsilyl)-1H-indol-3-yl)methoxy)methyl)-N,N-dimethylcyclohexanamine (polar diastereomer)

In a flask with a Schlenk attachment a mixture of Ain-07 (540 mg, 1.35 mmol), 2-iodoaniline (355 mg, 1.62 mmol), sodium carbonate (715 mg, 6.75 mmol) and [1,3-bis-(2,6-diisopropylphenyl)imidazol-2-ylidene]-(3-chloropyridyl)palladium(II) dichloride (PEPPSI, 136 mg, 0.2 mmol) was evacuated, flushed with argon and then N,N-dimethyl formamide (7 ml) was added. The reaction mixture was stirred for 22 h at 100° C. and then concentrated to small volume under vacuum. Toluene was added to the residue, which was concentrated to dryness again and then diethyl ether (20 ml) was added. The mixture was washed with 1 M sodium thiosultate solution (10 ml). The precipitated deposit was filtered off. The organic phase was washed with sodium chloride solution (10 ml) and water (10 ml), dried with sodium sulfate and concentrated to small volume under vacuum. The crude product (590 mg) was purified by flash chromatography (30 g, 20×2.5 cm) with methanol and then purified again by flash chromatography (40 g, 14×4 cm) with ethyl acetate/methanol (9:1).

Yield: 240 mg (36%), cream-coloured solid

Melting point: 46-48° C.

¹H-NMR (DMSO-d₆): 0.35 (s, 6H); 0.87 (s, 9H); 1.11-1.75 (m, 9H); 2.24 (br s, 6H); 2.67 (s, 2H); 3.23 (d, J=5.9 Hz, 2H); 4.53 (s, 2H); 6.91-7.02 (m, 1H); 7.03-7.12 (m, 1H); 7.31-7.12 (m, 5H); 7.40 (d, J=8.1 Hz, 1H); 7.52 (d, J=7.8 Hz, 1H); 10.67 (s, 1H).

### Example 36

### 4-(((1H-Indol-3-yl)methoxy)methyl)-1-benzyl-N,N-dimethylcyclohexanamine (polar diastereomer)

A mixture of benzyltrimethyl ammonium fluoride monohydrate (315 mg, 1.68 mmol) and activated molecular sieve 4 Å (approx. 1.2 g) in anhydrous tetrahydrofuran (8 ml) was stirred for 1 h at room temperature. A solution of Example 34 (275 mg, 0.56 mmol) in anhydrous tetrahydrofuran (4 ml) was then added to the mixture and the mixture was refluxed for 2 h. Further activated molecular sieve 4 Å (300 mg) was added to the reaction mixture and the mixture was refluxed for a further 1.5 h. The reaction mixture was filtered, the filter residue washed with tetrahydrofuran, the filtrate concentrated to small volume under vacuum and the residue (260 mg) purified by flash chromatography (20 g, 22×2.0 cm) with ethyl acetate/methanol (4:1).

Yield: 81 mg (38%), yellowish solid

Melting point: 44-50° C.

¹H-NMR (DMSO-d₆): 1.18-1.79 (m, 9H); 2.28 (s, 6H); 2.74 (br s, 2H); 3.21 (d, J=6.4 Hz, 2H); 4.54 (s, 2H); 6.95-7.13 (m, 2H); 7.13-7.40 (m, 7H); 7.53 (d, J=7.8 Hz, 1H); 10.99 (s, 1H). In some cases a double set of signals is observed; the impurity could be either the corresponding monomethyl amino derivative or the hydrochloride of the monomethyl or dimethyl amino derivative.

¹³C-NMR (DMSO-d₆): 24.0 (br); 28.4 (br); 30.0; 34.8; 356; 36.3; 37.2; 64.4; 72.4; 73.2; 11.4; 11.9; 118.6; 121.1; 124.5, 125.9 (br); 126.9; 127.7 (br); 130.5; 130.6; 136.3.

### Example 37

### 1-Benzyl-4-(((5-fluoro-2-(triethylsilyl)-1H-indol-3-yl)methoxy)methyl)-N,N-dimethylcyclohexanamine (polar diastereomer)

In a flask with a Schlenk attachment a mixture of Ain-06 (550 mg, 1.38 mmol), 4-fluoro-2-iodoaniline (360 mg, 1.52 mmol), sodium carbonate (731 mg, 6.9 mmol) and [1,3-bis-(2,6-diisopropylphenyl)imidazol-2-ylidene]-(3-chloropyridyl)palladium(II) dichloride (PEPPSI, 141 mg, 0.21 mmol) was evacuated, flushed with argon and then N,N-dimethyl formamide (8 ml) was added. The reaction mixture was stirred for 18 h at 110° C. and then concentrated to small volume under vacuum. Toluene was added to the residue, which was concentrated to dryness again and then diethyl ether (50 ml) was added. The solution was washed with 1 M sodium thiosulfate solution (50 ml), sodium chloride solution (50 ml) and water (50 ml), dried with sodium sulfate and concentrated to small volume under vacuum. The crude product (682 mg) was purified by flash chromatography (84 g, 19×4 cm) with chloroform/methanol (95:5).

Yield: 432 mg (61%), oil

¹H-NMR (DMSO-d₆): 0.78-0.99 (m, 15H); 1.18-1.53 (m, 5H); 1-54-1.75 (m, 4H); 2.24 (s, 6H); 2.67 (s, 2H); 3.19 (d, J=6.3 Hz, 2H); 4.50 (s, 2H); 6.88-6.98 (m, 1H); 7.10-7.35 (m, 5H); 7.35-7.42 (m, 2H); 10.75 (s, 1H). In some cases a double set of signals is observed; the impurity could be either the corresponding monomethyl amino derivative or the hydrochloride of the monomethyl or dimethyl amino derivative.

¹³C-NMR (DMSO-d₆): 3.1, 7.2; 23.6; 24.4; 28.0; 34.4; 35.7; 36.3; 37.1; 64.3; 72.3; 73.6; 102.8 (d, J=23 Hz); 109.6 (d, J=26 Hz); 112.1 (d, J=10 Hz); 121.3 (d, J=5 Hz); 125.5; 127.5; 128.4; 128.5; 130.4; 135.3; 135.8; 156.7 (d, J=232 Hz).

### Example 38

### 1-Benzyl-4-(((2-(tert-butyldimethylsilyl)-5-fluoro-1H-indol-3-yl)methoxy)methyl)-N,N-dimethylcyclohexanamine (polar diastereomer)

In a heated flask with a Schlenk attachment a mixture of Ain-07 (400 mg, 1 mmol), 4-fluoro-2-iodoaniline (284 mg, 1.2 mmol), sodium carbonate (530 mg, 5 mmol) and [1,3-bis-(2,6-diisopropylphenyl)imidazol-2-ylidene]-(3-chloropyridyl)palladium(II) dichloride (PEPPSI, 136 mg, 0.2 mmol) was evacuated, flushed with argon and then N,N-dimethyl formamide (6 ml) was added. The reaction mixture was stirred for 20 h at 100° C. and then concentrated to small volume under vacuum. Toluene was added to the residue, which was concentrated to dryness again and then diethyl ether (40 ml) was added. The mixture was washed with 1 M sodium thiosulfate solution (10 ml). The precipitated deposit was filtered off. The organic phase was washed with sodium chloride solution (10 ml) and water (10 ml), dried with sodium sulfate and concentrated to small volume under vacuum. The crude product (460 mg) was purified by flash chromatography (39 g, 22×2.5 cm) and then purified again by flash chromatography (30 g, 18×2.5 cm), with methanol in each case.

Yield: 289 mg (53%), yellowish solid

Melting point: 50-55° C.

¹H-NMR (DMSO-d₆): 0.34 (s, 6H); 0.86 (s, 9H); 1.16-1.52 (m, 6H); 1.56-1.75 (m, 3H); 2.22 (s, 6H); 2.65 (s, 2H); 3.14-3.27 (m, 2H); 4.49 (s, 2H); 6.93 (dt, J=9.2 Hz, 1H); 7.10-7.26 (m, 6H); 7.39 (dd, J=8.8, 4.2 Hz, 1H); 10.77 (s, 1H).

¹³C-NMR (DMSO-d₆): −5.2; 16.9; 23.5; 26.3; 28.0; 34.3; 35.8; 37.1; 57.7; 64.6; 72.5; 102.9 (d, J=23 Hz); 109.7 (d, J=26 Hz); 112.1 (d, J=10 Hz); 121.5 (d, J=5 Hz); 125.5; 127.5; 128.6; 130.4; 135.3; 136.0; 139.0; 156.7 (d, J=232 Hz).

### Example 39

### 1-Benzyl-4-(((5-fluoro-1H-indol-3-yl)methoxy)methyl)-N,N-dimethylcyclohexanamine (polar diastereomer)

A mixture of benzyltrimethyl ammonium fluoride monohydrate (276 mg, 1.47 mmol) and activated molecular sieve 4 Å (approx. 1 g) in anhydrous tetrahydrofuran (7 ml) was stirred for 1 h at room temperature. A solution of Example 37 (250 mg, 0.49 mmol) in anhydrous tetrahydrofuran (3 ml) was then added to the mixture and the mixture was refluxed for 2 h. The reaction mixture was filtered, the filter residue washed with tetrahydrofuran, the filtrate concentrated to small volume under vacuum and the residue (200 mg) purified by flash chromatography (20 g, 21×2.0 cm) with ethyl acetate/methanol (9:1).

Yield: 48 mg (25%), yellowish oil

¹H-NMR (DMSO-d₆): 1.20-1-52 (m, 5H); 1.52-1.73 (m, 4H); 2.23 (s, 6H); 2.68 (s, 2H); 3.19 (d, J=6.3 Hz, 2H); 4.50 (s, 2H); 6.89-6.97 (m, 1H); 7.11-7.40 (m, 8H); 11.09 (s, 1H).

¹³C-NMR (DMSO-d₆): 23.8; 28.2; 34.6; 35.8; 37.2; 57.7; 64.2; 72.2; 103.2 (d, J=23 Hz); 109.2 (d, J=26 Hz); 112.2 (d, J=4 Hz); 112.2 (d, J=10 Hz); 125.5; 126.5; 127.2; 127.5; 130.4; 133.0; 139.0; 156.7 (d, J=231 Hz).

### Example 40

### 1-Benzyl-N,N-dimethyl-4-(((2-(triethylsilyl)-1H-pyrrolo[2,3-b]pyridin-3-yl)methoxy)methyl)cyclohexanamine hydrochloride (polar diastereomer)

In a flask with a Schlenk attachment a mixture of Ain-06 (500 mg, 1.25 mmol), 2-amino-3-iodopyridine (302 mg, 1.38 mmol), sodium carbonate (662 mg, 6.25 mmol) and [1,3-bis-(2,6-diisopropylphenyl)imidazol-2-ylidene]-(3-chloropyridyl)palladium(II) dichloride (PEPPSI, 127 mg, 0.19 mmol) was evacuated, flushed with argon and then N,N-dimethyl formamide (7 ml) was added. The reaction mixture was stirred for 20 h at 110° C. and then concentrated to small volume under vacuum. Toluene was added to the residue, which was concentrated to dryness again and then ethyl acetate (40 ml) was added. The mixture was washed with 1 M sodium thiosulfate solution, sodium chloride solution and water (20 ml each), dried with sodium sulfate and concentrated to small volume under vacuum. The crude product (665 mg) was purified by flash chromatography (100 g, 23×4 cm) with chloroform/methanol (9:1).

Yield: 590 mg (88%), beige-coloured solid

Melting point: 62-67° C.

¹H-NMR (DMSO-d₆): 0.82-1.01 (m, 15H); 1.18-1.44 (m, 4H); 1.49-1.84 (m, 5H); 2.56 (s, 6H); 3.08 (s, 2H); 3.24 (s, 2H); 4.58 (s, 2H); 7.02-7.08 (m, 1H); 7.17-7.41 (m, 5H); 7.90-8.00 (m, 1H); 8.22-8.26 (m, 1H); 9.83 (br s, 1H); 11.31 (s, 1H).

¹³C-NMR (DMSO-d₆): 3.0 (3C); 7.2 (3C); 24.5 (2C); 29.9 (2C); 43.5; 35.4; 37.3 (2C); 64.3; 66.6; 73.2; 115.0; 120.2; 120.3; 126.6; 127.1; 128.5; 130.5; 134.5; 135.5; 143.1; 150.7.

### Example 41

### 4-(((1H-Pyrrolo[2,3-b]pyridin-3-yl)methoxy)methyl)-1-benzyl-N,N-dimethylcyclohexanamine (polar diastereomer)

A mixture of benzyltrimethyl ammonium fluoride monohydrate (466 mg, 2.49 mmol) and activated molecular sieve 4 Å (approx. 1 g) in anhydrous tetrahydrofuran (10 ml) was stirred for 1 h at room temperature. A solution of Example 40 (440 mg, 0.83 mmol) in anhydrous tetrahydrofuran (5 ml) was then added to the mixture and the mixture was refluxed for 2.5 h. The reaction mixture was filtered, the filter residue washed with tetrahydrofuran, the filtrate concentrated to small volume under vacuum and the residue (270 mg) purified by flash chromatography (24 g, 18×2.5 cm) with ethyl acetate/methanol (4:1).

Yield: 76 mg (24%), yellow oil

¹H-NMR (DMSO-d₆): 1.19-1.74 (m, 9H); 2.23 (br s, 6H); 2.66 (s, 2H); 3.19 (d, J=6.23 Hz, 2H); 4.54 (s, 2H); 7.05 (dd, J=7.8 and 4.6 Hz, 1H); 7.08-7.31 (m, 5H); 7.38-7.40 (m, 1H); 7.91 (d, J=7.6 Hz, 1H); 8.22 (dd, J=4.6 and 1.5 Hz, 1H); 11.49 (s, 1H).

### Example 42

### 1-Benzyl-4-(((2-(tert-butyldimethylsilyl)-1H-pyrrolo[3,2-c]pyridin-3-yl)methoxy)methyl)-N,N-dimethylcyclohexanamine (polar diastereomer)

In a flask with a Schlenk attachment a mixture of Ain-07 (480 mg, 1.2 mmol), 4-amino-3-iodopyridine (317 mg, 1.44 mmol), sodium carbonate (636 mg, 6 mmol) and [1,3-bis-(2,6-diisopropylphenyl)imidazol-2-ylidene]-(3-chloropyridyl)palladium(II) dichloride (PEPPSI, 122 mg, 0.18 mmol) was evacuated, flushed with argon and then N,N-dimethyl formamide (6 ml) was added. The reaction mixture was stirred for 3 h at 120° C. and 22 h at 130° C. and then concentrated to small volume under vacuum. Toluene was added twice to the residue, which was concentrated to dryness again each time, and then diethyl ether (20 ml) was added. The mixture was washed with 1 M sodium thiosultate solution (10 ml). The precipitated deposit was filtered off. The organic phase was washed with sodium chloride solution (10 ml) and water (10 ml), dried with sodium sulfate and concentrated to small volume under vacuum. The crude product (530 mg) was purified by flash chromatography (45 g, 15×4 cm) with ethyl acetate/methanol (9:1→4:1).

Yield: 253 mg (43%)

Melting point: not determinable

¹H-NMR (DMSO-d₆): 0.37 (s, 6H); 0.86 (s, 9H); 1.20-1.75 (m, 9H); 2.28 (s, 6H); 2.74 (s, 2H); 3.25 (d, J=6.3 Hz, 2H); 4.60 (s, 2H); 7.11-7.30 (m, 5H); 7.37 (d, J=5.7 Hz, 1H), 8.15 (d, J=5.8 Hz, 1H), 8.85 (s, 1H); 11.11 (s, 1H).

¹³C-NMR (DMSO-d₆): −5.1; 17.0; 23.7; 26.3; 28.4; 34.5; 35.5; 37.1; 59.7; 64.5; 72.8; 106.5; 121.7; 125.2; 125.8; 127.7; 130.4; 135.2; 138.2; 140.0; 141.8; 142.0.

### Example 43

### 1-Butyl-N,N-dimethyl-4-(((2-(triethylsilyl)-1H-indol-3-yl)methoxy)methyl)cyclohexanamine (non-polar diastereomer)

In a flask with a Schlenk attachment a mixture of Ain-08 (365 mg, 1.0 mmol), 2-iodoaniline (261 mg, 1.2 mmol), [1,3-bis-(2,6-diisopropylphenyl)imidazol-2-ylidene]-3-(chloropyridyl)palladium(II) chloride (PEPPSI™, 136 mg, 0.2 mmol) and sodium carbonate (529 mg, 5 mmol) was evacuated for 30 min. N,N-Dimethyl formamide (8 ml), which had previously been saturated with argon for 1 h, was then added under an argon stream. The suspension was stirred overnight at 100° C. and then concentrated to small volume under vacuum. Toluene (3×30 ml) was repeatedly added to the residue, which was concentrated to small volume under vacuum again each time and taken up in water (20 ml) and ethyl acetate (20 ml). The phases were separated and the aqueous phase was extracted with ethyl acetate (2×20 ml). The combined organic phases were washed with 1 M sodium thiosultate solution (30 ml) and saturated sodium chloride solution (50 ml) and concentrated to small volume under vacuum. The crude product (617 mg) was purified by flash chromatography (70 g, 20×3.6 cm) with ethyl acetate/methanol (9:1).

Yield: 256 mg (56%), brown oil

¹H-NMR (DMSO-d₆): 0.81-1.00 (m, 18H); 1.08-1.90 (m, 15H), 2.10 (s, 4H); 2.62 (s, 2H); 3.25 (s, 2H); 4.57 and 4.62 (s, 2H); 6.98 (t, 1H, J=7.4 Hz); 7.08 (t, 1H, J=7.4 Hz); 7.40 (d, 1H, J=8.1 Hz); 7.56 (d, 1H, J=7.7 Hz); 8.76 (s, 0.3H); 10.69 (s, 1H).

¹³C-NMR (DMSO-d₆): 3.1; 3.7; 7.2; 22.7; 23.3; 24.1; 24.9; 25.4; 26.2; 28.1; 30.6; 31.5; 35.0; 36.6; 37.5; 55.6; 64.4; 66.5; 73.3; 75.1; 111.2; 118.4; 121.3; 128.5; 133.3; 138.6

### Example 44

### 4-(((1H-Indol-3-yl)methoxy)methyl)-1-butyl-N,N-dimethylcyclohexanamine (non-polar diastereomer)

A suspension of benzyltrimethyl ammonium fluoride monohydrate (323 mg, 1.72 mmol) and activated molecular sieve 4 Å (2 g) in anhydrous tetrahydrofuran (10 ml) was stirred for 1 h at room temperature. A solution of Example 43 (273 mg, 0.57 mmol) in anhydrous tetrahydrofuran (15 ml) was then added to the mixture and the mixture was refluxed whilst stirring for 1.5 h. Then the mixture was filtered and the filter residue was washed with tetrahydrofuran (2×35 ml). The filtrate was concentrated to small volume under vacuum and the crude product (246 mg) purified by flash chromatography (30 g, 20×2.7 cm) with ethyl acetate/cyclohexane (9:1).

Yield: 137 mg (67%), yellowish oil

¹H-NMR (DMSO-d₆): 0.87 (t, 3H, J=7.0 Hz); 1.10-1.75 (m, 15H); 2.10 (br s, 6H); 3.22 (d, 2H, J=5.9 Hz); 4.58 (s, 2H); 6.99 (dt, 1H, J=7.0, 1.1 Hz); 7.08 (dt, 1H, J=7.0, 1.2 Hz); 7.30 (d, 1H, J=2.4 Hz); 7.36 (td, J=8.0, 0.9 Hz); 7.56 (br d, 1H, J=7.2 Hz); 11.01 (s, 1H).

¹³C-NMR (DMSO-d₆): 14.0; 23.3; 24.1; 26.3; 30.6; 31.5; 36.7; 37.5; 55.7; 64.4; 74.1; 111.4; 112.0; 118.6; 118.7; 121.1; 124.6; 127.0; 136.3.

### Example 45

### 1-Butyl-4-(((5-fluoro-2-(triethylsilyl)-1H-indol-3-yl)methoxy)methyl)-N,N-dimethylcyclohexanamine (non-polar diastereomer)

In a flask with a Schlenk attachment a mixture of Ain-08 (548 mg, 1.5 mmol), 4-fluoro-2-iodoaniline (426 mg, 1.8 mmol), [1,3-bis-(2,6-diisopropylphenyl)imidazol-2-ylidene]-3-(chloropyridyl)palladium(II) chloride (PEPPSI™, 204 mg, 0.3 mmol) and sodium carbonate (793 mg, 7.48 mmol) was evacuated for 30 min. N,N-Dimethyl formamide (8 ml), which had previously been saturated with argon for 1 h, was then added under an argon stream. The suspension was stirred overnight at 120° C. and then concentrated to small volume under vacuum. Toluene (3×30 ml) was repeatedly added to the residue, which was concentrated to small volume under vacuum again each time and taken up in water (20 ml) and ethyl acetate (20 ml). The phases were separated and the aqueous phase was extracted with ethyl acetate (2×20 ml). The combined organic phases were washed with 1 M sodium thiosulfate solution (30 ml) and saturated sodium chloride solution (50 ml) and concentrated to small volume under vacuum. The crude product (932 mg) was purified by flash chromatography (100 g, 3.6×25 cm) with ethyl acetate/methanol (9:1). Since no pure product could be isolated, the corresponding mixed fractions (472 mg) were purified by MPLC [130 g, LiChroprep Si60 (15-25 μm) 46×2.6 cm] with ethyl acetate/methanol (9:1).

Yield: 343 mg (48%), brown oil

¹H-NMR (DMSO-d₆): 0.82-0.98 (m, 18H); 1.05-1.55 (m, 13H); 1.65 (d, 2H, J=8.6 Hz); 2.10 (s, 6H); 3.24 (d, 2H, J=6.2 Hz); 4.55 (s, 2H); 6.92 (dt, 1H, J=9.1, 2.5 Hz); 7.26 (dd, 1H, J=2.5 and 10.0 Hz); 7.37 (dd, 1H, J=4.6 and 8.8 Hz); 10.76 (s, 1H).

¹³C-NMR (DMSO-d₆): 3.0; 7.2; 13.9; 23.3; 24.0; 26.3; 30.6; 31.5; 36.6; 37.5; 55.6; 64.3; 75.1; 102.8 (d, J=22 Hz); 110 (d, J=26 Hz); 112.2; 121.5; 128.6 (d, J=10 Hz); 135.3; 135.8; 156.7 (d, J=232 Hz).

### Example 46

### 1-Butyl-4-(((5-fluoro-1H-indol-3-yl)methoxy)methyl)-N,N-dimethylcyclohexanamine (non-polar diastereomer)

A suspension of benzyltrimethyl ammonium fluoride monohydrate (334 mg, 1.78 mmol) and activated molecular sieve 4 Å (2 g) in anhydrous tetrahydrofuran (10 ml) was stirred for 1 h at room temperature. A solution of Example 45 (283 mg, 0.59 mmol) in anhydrous tetrahydrofuran (20 ml) was then added to the mixture and the suspension was refluxed whilst stirring for 2 h. Then the mixture was filtered and the filter residue was washed with tetrahydrofuran (2×35 ml). The filtrate was concentrated to small volume under vacuum and the crude product (246 mg) purified by flash chromatography (30 g, 20×2.5 cm) with ethyl acetate/methanol (9:1).

Yield: 148 mg (68%), yellowish oil

¹H-NMR (DMSO-d₆): 0.87 (t, 3H, J=6.8 Hz); 1.00-1.60 (m, 15H); 2.09 (br s, 5H); 2.62 (br s, 1H); 3.21 (d, 2H, J=5.7 Hz); 4.55 (s, 2H); 6.92 (dt, 1H, J=9.3 and 2.6 Hz); 7.26 (dd, 1H, J=10.0 and 2.6 Hz); 7.32-7.40 (m, 2H); 7.90 (brs, 0.2H, Me₂N+H signal); 11.09 and 11.17 (2 s, 1H).

¹³C-NMR (DMSO-d₆): 14.0; 23.3; 24.1; 26.3; 30.7; 31.5; 36.7; 37.4; 55.7; 64.2; 74.7; 103.2 (d, J=24 Hz); 109.2 (d, J=27 Hz); 112.3 (d, J=9 Hz); 127.2 (d, J=10 Hz); 133.0; 156.7 (d, J=231 Hz).

### Example 47

### 1-Butyl-N,N-dimethyl-4-(((2-(triethylsilyl)-1H-pyrrolo[2,3-b]pyridin-3-yl)methoxy)methyl)cyclohexanamine (non-polar diastereomer)

In a flask with a Schlenk attachment a mixture of Ain-08 (548 mg, 1.5 mmol), 2-amino-3-iodopyridine (393 mg, 1.78 mmol), [1,3-bis-(2,6-diisopropylphenyl)imidazol-2-ylidene]-3-(chloropyridyl)palladium(II) chloride (PEPPSI™, 204 mg, 0.3 mmol) and sodium carbonate (794 mg, 7.50 mmol) was evacuated for 30 min. N,N-Dimethyl formamide (8 ml), which had previously been saturated with argon for 1 h, was then added under an argon stream. The suspension was stirred overnight at 120° C. and then concentrated to small volume under vacuum. Toluene (3×30 ml) was repeatedly added to the residue, which was concentrated to small volume under vacuum again each time and taken up in water (20 ml) and ethyl acetate (20 ml). The phases were separated and the aqueous phase was extracted with ethyl acetate (2×20 ml). The combined organic phases were washed with 1 M sodium thiosultate solution (30 ml) and saturated sodium chloride solution (50 ml) and concentrated to small volume under vacuum. The crude product (966 mg) was purified by flash chromatography (100 g, 20×3.6 cm) with ethyl acetate/methanol (9:1).

Yield: 475 mg (70%), yellow oil

¹H-NMR (DMSO-d₆): 0.82-0.94 (m, 18H); 1.20-1.56 (m, 13H); 1.65 (br d, 2H, J=8.5 Hz); 2.09 (s, 6H); 3.24 (d, 2H, J=6.0 Hz); 4.58 (s, 2H); 7.04 (dd, 1H, J=4.6 and 7.8 Hz); 7.96 (dd, 1H, J=1.4 and 7.8 Hz); 8.22 (dd, 1H, J=1.5 and 4.6 Hz); 11.28 (s, 1H).

¹³C-NMR (DMSO-d₆): 3.0; 7.2; 14.0; 23.3; 24.1; 26.3; 26.5; 30.7; 31.5; 36.6; 37.5; 55.6; 60.0; 64.3; 75.1; 115.0; 120.4; 126.7; 134.4; 143.0; 150.7.

### Example 48

### 4-(((1H-Pyrrolo[2,3-b]pyridin-3-yl)methoxy)methyl)-1-butyl-N,N-dimethylcyclohexanamine (non-polar diastereomer)

A suspension of benzyltrimethyl ammonium fluoride monohydrate (468 mg, 2.49 mmol) and activated molecular sieve 4 Å (2 g) in anhydrous tetrahydrofuran (20 ml) was stirred for 1 h at room temperature. A solution of Example 47 (396 mg, 0.86 mmol) in anhydrous tetrahydrofuran (10 ml) was then added to the mixture and the suspension was refluxed whilst stirring for 2 h. Then the mixture was filtered and the filter residue was washed with tetrahydrofuran (2×35 ml). The filtrate was concentrated to small volume under vacuum and the crude product (398 mg) purified by flash chromatography (40 g, 20×2.5 cm) with methanol.

Yield: 178 mg (60%), colourless oil

¹H-NMR (DMSO-d₆): 0.86 (t, 3H, J=7.1 Hz); 1.06-1.54 (m, 13H); 1.64 (br d, 2H, J=11.6 Hz); 2.09 (s, 6H); 3.21 (d, 2H, J=6.3 Hz); 4.57 (s, 2H); 7.05 (dd, 1H, J=4.7 and 7.8 Hz); 7.40 (d, 1H, 2.4 Hz); 7.95 (dd, 1H, 1.4 and 7.8 Hz); 8.20 (dd, 1H, J=1.4 and 4.7 Hz); 11.49 (s, 1H).

¹³C-NMR (DMSO-d₆): 13.9; 23.3; 23.8; 24.0; 26.3; 30.7; 31.5; 36.6; 37.4; 54.8; 55.7; 64.4; 74.8; 111.0; 115.2; 119.1; 124.9; 126.9; 142.6; 148.7.

### Example 49

### 1-Butyl-N,N-dimethyl-4-(((2-(triethylsilyl)-1H-pyrrolo[3,2-c]pyridin-3-yl)methoxy)methyl)cyclohexanamine (non-polar diastereomer)

In a flask with a Schlenk attachment a mixture of Ain-08 (548 mg, 1.50 mmol), 4-amino-3-iodopyridine (391 mg, 1.78 mmol), [1,3-bis-(2,6-diisopropylphenyl)imidazol-2-ylidene]-3-(chloropyridyl)palladium(II) chloride (PEPPSI™, 204 mg, 0.30 mmol) and sodium carbonate (794 mg, 7.50 mmol) was evacuated for 30 min. N,N-Dimethyl formamide (8 ml), which had previously been saturated with argon for 1 h, was then added under an argon stream. The suspension was stirred overnight at 120° C. and then concentrated to small volume under vacuum. Toluene (3×30 ml) was repeatedly added to the residue, which was concentrated to small volume under vacuum again each time and then taken up in water (20 ml) and ethyl acetate (20 ml). The phases were separated and the aqueous phase was extracted with ethyl acetate (2×20 ml). The combined organic phases were washed with 1 M sodium thiosultate solution (30 ml) and saturated sodium chloride solution (50 ml) and concentrated to small volume under vacuum. The crude product (940 mg) was purified by flash chromatography (100 g, 20×3.6 cm) with ethyl acetate/methanol (1:1).

Yield: 428 mg (62%), yellowish oil

¹H-NMR (DMSO-d₆): 0.84-0.95 (m, 18H); 1.10-1.60 (m, 13H); 1.69 (br d, 2H, J=5.6 Hz); 2.20 (s, 6H); 3.29 (d, 2H, J=6.2 Hz); 4.65 (s, 2H); 7.35 (d, 1H, J=5.6 Hz); 8.14 (d, 1H, J=5.6 Hz); 8.86 (s, 1H); 11.07 (s, 1H).

¹³C-NMR (DMSO-d₆): 3.1; 7.2; 13.9; 23.2; 23.7; 26.1; 30.6; 36.6; 37.0; 64.2; 74.9; 106.6; 121.6; 125.3; 134.8; 140.0; 141.8; 142.0.

### Example 50

### 4-(((1H-Pyrrolo[3,2-c]pyridin-3-yl)methoxy)methyl)-1-butyl-N,N-dimethylcyclohexanamine (non-polar diastereomer)

A suspension of benzyltrimethyl ammonium fluoride monohydrate (431 mg, 2.30 mmol) and activated molecular sieve 4 Å (2 g) in anhydrous tetrahydrofuran (15 ml) was stirred for 1 h at room temperature. A solution of Example 49 (349 mg, 0.76 mmol) in anhydrous tetrahydrofuran (20 ml) was then added to the mixture and the suspension was refluxed whilst stirring for 2 h. Then the mixture was filtered and the filter residue was washed with tetrahydrofuran (2×35 ml). The filtrate was concentrated to small volume under vacuum and the crude product (313 mg) purified by flash chromatography (32 g, 20×2.5 cm) with methanol.

Yield: 160 mg (61%), colourless oil

¹H-NMR (DMSO-d₆): 0.87 (t, 3H, J=7.1 Hz); 1.20-1.60 (m, 13 Hz); 1.66 (br d, 2H, J=10.5 Hz); 2.14 (s, 6H); 3.24 (d, 2H, J=6.3 Hz); 4.63 (s, 2H); 7.34 (d, 1H, J=5.6 Hz); 7.38 (s, 1H); 8.16 (d, 1H, J=5.6 Hz); 8.84 (s, 1H) 11.37 (s, 1H).

¹³C-NMR (DMSO-d₆): 14.0; 23.3; 23.9; 26.2; 30.6; 31.2; 36.7; 37.2; 64.1; 74.7; 106.8; 112.1; 123.8; 125.3; 127.1; 128.6; 139.7; 140.1; 142.0.

### Example 51

### 1-Butyl-4-(((5-fluoro-2-(triethylsilyl)-1H-indol-3-yl)methoxy)methyl)-N-methylcyclohexanamine (non-polar diastereomer)

In a flask with a Schlenk attachment a mixture of Ain-08 (548 mg, 1.5 mmol), 4-fluoro-2-iodoaniline (426 mg, 1.8 mmol), [1,3-bis-(2,6-diisopropylphenyl)imidazol-2-ylidene]-3-(chloropyridyl)palladium(II) chloride (PEPPSI™, 204 mg, 0.3 mmol) and sodium carbonate (793 mg, 7.48 mmol) was evacuated for 30 min. N,N-Dimethyl formamide (8 ml), which had previously been saturated with argon for 1 h, was then added under an argon stream. The suspension was stirred overnight at 120° C. and then concentrated to small volume under vacuum. Toluene (3×30 ml) was repeatedly added to the residue, which was concentrated to small volume under vacuum again each time and taken up in water (20 ml) and ethyl acetate (20 ml). The phases were separated and the aqueous phase was extracted with ethyl acetate (2×20 ml). The combined organic phases were washed with 1 M sodium thiosulfate solution (30 ml) and saturated sodium chloride solution (50 ml) and concentrated to small volume under vacuum. The crude product (932 mg) was purified by flash chromatography (100 g, 3.6×25 cm) with ethyl acetate/methanol (9:1). Since no pure product could be isolated, the corresponding mixed fractions (472 mg) were purified by MPLC [130 g, LiChroprep Si60 (15-25 μm) 46×2.6 cm] with ethyl acetate/methanol (9:1).

Yield: 70 mg (10%), brown oil

¹H-NMR (DMSO-d₆): 0.83-0.97 (m, 18H); 1.03-1.29 (m, 14H); 1.56 (d, 2H, J=12.5 Hz); 2.06 (s, 3H); 3.24 (d, 2H, J=6.2 Hz); 4.55 (s, 2H); 6.92 (dt, 1H, J=2.5 and 9.1 Hz); 7.25 (dd, 1H, J=2.4 and 10.0 Hz); 7.38 (dd, 1H, J=4.6 and 8.8 Hz); 10.74 (s, 1H).

¹³C-NMR (DMSO-d₆): 3.1; 7.2; 14.0; 22.8; 24.1; 24.2; 26.8; 33.1; 37.4; 53.0; 64.4; 74.9; 102.8 (d, J=23 Hz); 109.6 (d, J=26 Hz) 112.1 (d, J=10 Hz); 121.4; 128.7; 135.5; 135.8; 156.7 (d, J=231 Hz).

### Example 52

### 1-Butyl-N-methyl-4-(((2-(triethylsilyl)-1H-indol-3-yl)methoxy)methyl)cyclohexanamine (non-polar diastereomer)

In a flask with a Schlenk attachment a mixture of Ain-08 (WW318A non-polar diastereoisomer) (365 mg, 1.0 mmol), 2-iodoaniline (261 mg, 1.2 mmol), [1,3-bis-(2,6-diisopropylphenyl)imidazol-2-ylidene]-3-(chloropyridyl)palladium(II) chloride (PEPPSI™, 136 mg, 0.2 mmol) and sodium carbonate (529 mg, 5 mmol) was evacuated for 30 min. N,N-Dimethyl formamide (8 ml), which had previously been saturated with argon for 1 h, was then added under an argon stream. The suspension was stirred overnight at 120° C. and then concentrated to small volume under vacuum. Toluene (3×30 ml) was repeatedly added to the residue, which was concentrated to small volume under vacuum again each time and taken up in water (20 ml) and ethyl acetate (20 ml). The phases were separated and the aqueous phase was extracted with ethyl acetate (2×20 ml). The combined organic phases were washed with 1 M sodium thiosultate solution (30 ml) and saturated sodium chloride solution (50 ml) and concentrated to small volume under vacuum. The crude product (378 mg) was purified by MPLC [130 g, LiChroprep Si60 (15-25 μm), 46×2.6 cm] with ethyl acetate/methanol (9:1).

Yield: 52 mg (7%)

¹H-NMR (DMSO-d₆): 0.82-0.99 (m, 18H); 1.00-1.50 (m, 14H); 1.56 (br d, 2H, J=12.5 Hz); 2.06 (s, 3H); 3.25 (d, 2H, J=6.1 Hz); 4.59 (s, 2H); 6.97 (t, 1H, J=7.4 Hz); 7.07 (t, 1H, J=7.4 Hz); 7.40 (d, 1H, J=8.1 Hz); 7.55 (d, 1H, J=7.9 Hz); 10.63 (s, 1H).

¹³C-NMR (DMSO-d₆): 3.2; 7.3; 14.0; 22.8; 24.1; 24.2; 26.8; 28.9; 33.1; 37.4; 37.6; 53.0; 64.4; 74.9; 111.3; 118.5; 118.4; 121.3; 121.4; 128.5; 133.3; 138.7.

### Example 53

### 1-Butyl-N-methyl-4-(((2-(triethylsilyl)-5-(trifluoromethyl)-1H-indol-3-yl)methoxy)methyl)cyclohexanamine (non-polar diastereomer)

In a flask with a Schlenk attachment a mixture of Ain-08 (400 mg, 1.09 mmol), 4-amino-3-iodobenzotrifluoride (372 mg, 1.29 mmol), [1,3-bis-(2,6-diisopropylphenyl)imidazol-2-ylidene]-3-(chloropyridyl)palladium(II) chloride (PEPPSI™, 148 mg, 0.22 mmol) and sodium carbonate (579 mg, 5.46 mmol) was evacuated for 30 min. N,N-Dimethyl formamide (10 ml), which had previously been saturated with argon for 1 h, was then added under an argon stream. The suspension was stirred overnight at 120° C. and then concentrated to small volume under vacuum. Toluene (3×30 ml) was repeatedly added to the residue, which was concentrated to small volume under vacuum again each time and taken up in water (20 ml) and ethyl acetate (20 ml). The phases were separated and the aqueous phase was extracted with ethyl acetate (2×20 ml). The combined organic phases were washed with 1 M sodium thiosulfate solution (30 ml) and saturated sodium chloride solution (50 ml), concentrated to small volume under vacuum, and the crude product (709 mg) was purified by flash chromatography (100 g, 25×3.6 cm) with ethyl acetate/methanol (9:1).

Yield: 177 mg (30%), yellowish oil

¹H-NMR (DMSO-d₆): 0.82-1.00 (m, 18H); 1.12-1.52 (m, 14H); 1.71 (br d, 2H, J=11.5 Hz); 2.27 (s, 3H); 3.30 (d, 2H, J=6.5 Hz, overlaid by the HDO signal); 4.66 (s, 2H); 7.37 (dd, 1H, J=1.6 and 8.6 Hz); 7.58 (d, 1H, J=8.6 Hz); 7.92 (s, 1H); 11.12 (s, 1H).

### Example 54

### 1-Butyl-N-methyl-4-(((5-(trifluoromethyl)-1H-indol-3-yl)methoxy)methyl)cyclohexanamine (non-polar diastereomer)

A suspension of benzyltrimethyl ammonium fluoride monohydrate (176 mg, 0.93 mmol) and activated molecular sieve 4 Å (2 g) in anhydrous tetrahydrofuran (20 ml) was stirred for 1 h at room temperature. A solution of Example 53 (163 mg, 0.32 mmol) in anhydrous tetrahydrofuran (20 ml) was then added to the mixture and the suspension was refluxed whilst stirring for 2 h. The mixture was filtered and the filter residue was washed with tetrahydrofuran (2×35 ml). The filtrate was concentrated to small volume under vacuum and the crude product (86 mg) purified by flash chromatography (10 g, 20×1.1 cm) with methanol.

Yield: 76 mg (60%), yellow oil

¹H-NMR (DMSO-d₆): 0.83 (t, 3H, J=7.0 Hz); 1.00 (dt, 2H, J=13.1 and 3.5 Hz); 1.08-1.45 (m, 11H); 1.50 (br d, 2H, J=12.6 Hz); 1.99 (s, 3H); 3.20 (d, 2H, J=6.2 Hz); 4.60 (s, 2H); 7.35 (dd, 1H, J=1.6 and 8.6 Hz); 7.47 (d, 1H, J=1.7 Hz); 7.53 (d, 1H, J=8.6 Hz); 7.89 (s, 1H); 11.39 (s, 1H). 1H was unable to be identified.

¹³C-NMR (DMSO-d₆): 14.0; 22.9; 23.3; 24.24; 24.28; 26.3; 27.0; 31.5; 33.6; 36.5; 37.5; 38.0; 37.7 (d, J=46.8 Hz); 52.2; 64.1; 74.7; 112.2; 113.3; 116.4; 117.5; 119.5 (q, J=31 Hz); 124.2; 126.2; 126.7; 126.9; 137.9.

### Example 55

### 4-(((5-Fluoro-2-(triethylsilyl)-1H-indol-3-yl)methoxy)methyl)-N,N-dimethyl-1-(thiophen-2-yl)cyclohexanamine (one of two possible diastereomers)

A degassed mixture of Ain-09 (795 mg, 2 mmol), 4-fluoro-2-iodoaniline (567 mg, 2.4 mmol), [1,3-bis-(2,6-diisopropylphenyl)imidazol-2-ylidene]-(3-chloropyridyl)palladium(II) chloride (PEPPSI, 273 mg, 0.4 mmol) and sodium carbonate (1.06 g, 10 mmol) in anhydrous and oxygen-free N,N-dimethyl formamide (10 ml) was stirred for 18 h at 100° C. The solvent was then removed under vacuum and the residue divided between water and diethyl ether (20 ml each). The phases were separated and the aqueous phase was extracted with diethyl ether (3×10 ml). The combined organic phases were washed with water and 1 M sodium thiosulfate solution (20 ml each), dried with sodium sulfate and concentrated to small volume under vacuum. The crude product (1.30 g) was purified by flash chromatography (100 g, 20×4.0 cm) with ethyl acetate/cyclohexane (1:9).

Yield: 552 mg (55%), brownish oil

¹H-NMR (DMSO-d₆): 0.82-0.98 (m, 15H); 1.30-1.70 (m, 7H); 1.96 (s, 6H); 2.37 (br d, 2H); 3.25-3.33 (m, 2H); 4.58 (s, 2H); 6.88-7.04 (m, 3H); 7.23-7.31 (m, 1H); 7.35-7.41 (m, 2H); 10.77 (s, 1H).

¹³C-NMR (DMSO-d₆): 3.1; 7.2; 24.4; 34.6; 37.1; 37.4; 58.2; 64.4; 74.7; 102.8 (d, J=23 Hz); 109.6 (d, J=27 Hz); 112.1 (d, J=10 Hz); 121.5; 123.0; 123.8; 126.0; 128.7 (d, J=10 Hz); 135.3; 135.9; 145.0; 156.7 (d, J=231 Hz).

### Example 56

### 4-(((5-Fluoro-1H-indol-3-yl)methoxy)methyl)-N,N-dimethyl-1-(thiophen-2-yl)cyclohexanamine (one of two possible diastereomers)

Activated molecular sieve 4 Å (2.00 g) was added to a suspension of benzyltrimethyl ammonium fluoride monohydrate (338 mg, 2 mmol) in anhydrous tetrahydrofuran (30 ml) and the mixture was stirred for 1 h at room temperature. A solution of Example 55 (320 mg, 0.64 mmol) in anhydrous tetrahydrofuran (20 ml) was then added to this suspension and the mixture was refluxed whilst stirring for 2 h. The reaction mixture was filtered, the filter residue washed with tetrahydrofuran, the filtrate concentrated to small volume under vacuum and the residue (320 mg) purified by flash chromatography (18 g, 20×2.0 cm) with ethyl acetate/cyclohexane (2:1).

Yield: 126 mg (51%), yellowish oil

¹H-NMR (DMSO-d₆): 1.30-1.70 (m, 7H); 1.97 (s, 6H); 2.38 (br d, 2H, J=11.1 Hz); 3.26 (d, 2H, J=6.3 Hz); 4.57 (s, 2H); 6.88-6.97 (m, 2H); 7.02 (dd, 1H, J=5.1, 3.5 Hz); 7.28 (dd, 1H, J=9.9, 2.6 Hz); 7.33-7.41 (m, 3H); 11.09 (s, 1H).

¹³C-NMR (DMSO-d₆): 24.4; 34.6; 37.0; 37.4; 58.3; 64.2; 74.3; 103.3 (d, J=23 Hz); 109.2 (d, J=26 Hz); 112.3 (d, J=5 Hz); 112.4, 123.0; 123.8; 126.0; 126.6; 127.3 (d, J=10 Hz); 132.9; 145.1; 156.7 (d, J=231 Hz).

### Example 57

### 3-(((4-(Azetidin-1-yl)-4-phenylcyclohexyl)methoxy)methyl)-5-fluoro-2-(triethylsilyl)-1H-indole (one of two possible diastereomers)

A mixture of Ain-10 (795 mg, 2 mmol), 4-fluoro-2-iodoaniline (567 mg, 2.4 mmol), [1,3-bis-(2,6-diisopropylphenyl)imidazol-2-ylidene]-(3-chloropyridyl)palladium(II) chloride (PEPPSI, 273 mg, 0.4 mmol) and sodium carbonate (1.06 g, 10 mmol) in anhydrous N,N-dimethyl formamide (10 ml) was stirred for 18 h at 100° C. The solvent was then removed under vacuum and the residue divided between water and diethyl ether (20 ml each). The aqueous phase was extracted with diethyl ether (3×10 ml) and the organic phase was washed with water and 1 M sodium thiosulfate solution (20 ml each). The combined organic phases were dried with sodium sulfate and concentrated to small volume under vacuum. The crude product (1.30 g) was purified by flash chromatography (100 g, 20×4.0 cm) with ethyl acetate/cyclohexane (1:9).

Yield: 707 mg (70%), brown oil

¹H-NMR (DMSO-d₆): 0.85-0.99 (m, 15H); 1.20-1.34 (m, 2H); 1.43-1.54 (m, 5H); 1.55-1.64 (m, 2H); 2.18 (d, 2H, J=13.0 Hz); 2.82 (t, 4H, J=6.8 Hz); 3.32-3.35 (m, 2H); 4.59 (s, 2H); 6.93 (dt, 1H, J=9.2, 2.5 Hz); 7.23-7.41 (m, 7H); 10.77 (s, 1H).

¹³C-NMR (DMSO-d₆): 3.1; 7.2; 15.5; 24.9; 26.3; 28.9; 30.4; 36.8; 45.8; 57.2; 64.4; 74.7; 102.8 (d, J=22 Hz); 109.6 (d, J=22 Hz); 112.2 (d, J=10 Hz); 121.5 (d, J=5 Hz); 126.3; 126.6; 127.4; 128.7 (d, J=10 Hz); 135.3; 135.9; 140.2; 156.8 (d, J=232 Hz).

### Example 58

### 3-(((4-(Azetidin-1-yl)-4-phenylcyclohexyl)methoxy)methyl)-5-fluoro-1H-indole (one of two possible diastereomers)

Activated molecular sieve (2.00 g) was added to a suspension of benzyltrimethyl ammonium fluoride monohydrate (148 mg, 0.84 mmol) in anhydrous tetrahydrofuran (20 ml) and the mixture was stirred for 1 h at room temperature. A solution of Example 57 (142 mg, 0.28 mmol) in anhydrous tetrahydrofuran (20 ml) was then added to this suspension and the mixture was refluxed whilst stirring for 1 h. The reaction mixture was then filtered, the filter residue washed with tetrahydrofuran, the filtrate concentrated to small volume under vacuum. The residue (73 mg) was purified by flash chromatography (10 g, 20×1.5 cm) with ethyl acetate/methanol (9:1).

Yield: 38 mg (34%), yellowish oil

¹H-NMR (CDCl₃): 1.48-1.78 (m, 9H); 2.09-2.16 (m, 2H); 3.01 (br s, 4H); 3.46 (d, 2H, J=6.4 Hz); 4.67 (s, 2H); 6.94 (dt, 1H, J=9.1, 2.5 Hz); 7.19 (d, 1H, J=2.5 Hz); 7.23-7.43 (m, 7H); 8.42 (s, 1H).

¹³C-NMR (CDCl₃): 16.1; 24.8; 30.0; 36.3; 46.6; 58.9; 65.1; 74.5; 104.2; 104.3 (d, J=24 Hz); 110.5 (d, J=27 Hz); 111.7 (d, J=10 Hz); 113.8 (d, J=5 Hz); 125.4; 126.8; 127.2; 127.7; 133.0; 139.4; 157.9 (d, J=235 Hz).

### Example 60

### 4-(((5-Fluoro-1-methyl-2-(triethylsilyl)-1H-indol-3-yl)methoxy)methyl)-N,N-dimethyl-1-phenylcyclohexanamine (one of two possible diastereomers)

A mixture of Ain-01 (578 mg, 1.5 mmol), [1,3-bis-(2,6-diisopropylphenyl)imidazol-2-ylidene]-(3-chloropyridyl)palladium(II) chloride (PEPPSI, 204 mg, 0.3 mmol), 4-fluoro-2-iodo-N-methylaniline (Ian-11) (594 mg, 1.9 mmol) and sodium carbonate (793 mg, 7.5 mmol) was evacuated for 30 min in an oil pump. Anhydrous N,N-dimethyl formamide (8 ml), which had previously been flushed with argon for 1 h, was then added via a Schlenk attachment. The reaction mixture was then stirred for 18 h at 100° C. and then concentrated to small volume under vacuum. Toluene was repeatedly added to the residue, which was concentrated to small volume under vacuum again each time and then divided between water and ethyl acetate (20 ml each). The phases were separated and the aqueous phase was extracted with ethyl acetate (3×30 ml). The combined organic phases were washed with 1 M sodium thiosulfate solution (30 ml) and saturated sodium chloride solution (50 ml). The combined organic phases were dried with sodium sulfate and concentrated to small volume under vacuum. The crude product (965 mg) was purified by flash chromatography (100 g, 20×3.7 cm) with dichloromethane/methanol (95:5).

Yield: 156 mg (21%), brown oil

¹H-NMR (DMSO-d₆): 0.86-1.04 (m, 14H); 1.20-1.70 (m, 9H); 1.88 (br s, 6H); 2.55 (br d, 1H, J=12.0 Hz); 3.34 (d, 2H, overlaid by the HDO signal); 3.82 (s, 3H); 4.57 (s, 2H); 7.02 (dt, 1H, J=2.5, 9.2 Hz); 7.23 (br d, 1H, J=4.4 Hz); 7.26-7.40 (m, 5H); 7.45 (dd, 1H, J=4.4, 8.9 Hz).

¹³C-NMR (DMSO-d₆): 3.6; 7.2; 24.4; 32.0; 33.2; 37.1; 37.3; 54.7; 58.4; 63.4; 74.5; 102.9 (d, J=23 Hz); 109.9 (d, J=25 Hz); 110.6 (d, J=10 Hz); 122.6; 126.0; 126.4; 127.2; 128.6 (d, J=10 Hz); 136.0; 137.8; 139.4; 157.0 (d, J=231 Hz).

### Example 61

### 4-(((5-Fluoro-1-methyl-1H-indol-3-yl)methoxy)methyl)-N,N-dimethyl-1-phenylcyclohexanamine (one of two possible diastereomers)

A suspension of benzyltrimethyl ammonium fluoride monohydrate (251 mg, 1.35 mmol) and molecular sieve 4 Å (2.0 g) in anhydrous tetrahydrofuran (50 ml) was stirred for 1 h at room temperature. A solution of Example 60 (220 mg, 0.432 mmol) in anhydrous tetrahydrofuran (60 ml) was then added to this suspension. The mixture was stirred for 6 h at 75° C. and then overnight at room temperature. The suspension was filtered, the filter residue washed with tetrahydrofuran (2×20 ml) and the filtrate concentrated to small volume under vacuum. The crude product (236 mg) was purified by flash chromatography (18 g, 20×1.6 cm) with dichloromethane/methanol (95:5).

Yield: 104 mg (61%), yellowish oil

¹H-NMR (DMSO-d₆): 1.26-1.70 (m, 7H); 1.90 (s, 6H); 2.55 (br d, 2H, J=11.6 Hz); 3.29 (d, 2H, J=6.4 Hz); 3.76 (s, 3H); 4.56 (s, 2H); 7.00 (dt, 1H, J=2.6, 9.2 Hz); 7.22 (m, 1H); 7.28-7.36 (m, 5H); 7.38 (s, 1H); 7.42 (dd, 1H, J=4.4, 8.9 Hz).

¹³C-NMR (DMSO-d₆): 24.5; 32.0; 32.5; 36.8; 37.3; 58.6; 64.0; 74.2; 103.6 (d, J=23 Hz); 109.3 (d, J=26 Hz); 110.7 (d, J=10 Hz); 111.2; 111.4; 126.0; 126.4; 127.1; 127.5; 127.6; 130.6; 133.4; 139.4; 156.8 (d, J=231 Hz).

### Example 62

### 4-(((5-Fluoro-1-(methylsulfonyl)-1H-indol-3-yl)methoxy)methyl)-N,N-dimethyl-1-phenylcyclohexanamine (one of two possible diastereomers)

A suspension of benzyltrimethyl ammonium fluoride monohydrate (760 mg, 4.10 mmol) and molecular sieve 4 Å (4.0 g) in anhydrous tetrahydrofuran (60 ml) was stirred for 1 h at room temperature. A solution of Example 17 (760 mg, 1.33 mmol) in anhydrous tetrahydrofuran (60 ml) was then added to this suspension. The mixture was stirred for 1 h at 40° C. and then overnight at room temperature. The suspension was filtered, the filter residue washed with tetrahydrofuran (2×20 ml) and the filtrate concentrated to small volume under vacuum. The crude product (780 mg) was purified by flash chromatography (85 g, 3.6×20 cm) with cyclohexane/ethyl acetate (1:2).

Yield: 409 mg (67%), yellowish oil

¹H-NMR (DMSO-d₆): 1.22-1.70 (m, 7H); 1.89 (s, 6H); 2.58 (d, 2H, J=12.4 Hz); 3.35 (d, 2H, J=6.3 Hz); 3.44 (s, 3H); 4.62 (s, 2H); 7.18-7.27 (m, 2H); 7.28-7.40 (m, 4H); 7.47 (dd, 1H, J=2.5, 9.2 Hz); 7.65 (s, 1H); 7.84 (dd, 1H, J=4.5, 9.1 Hz).

¹³C-NMR (DMSO-d₆): 24.4; 32.0; 36.8; 37.3; 58.4; 63.6; 75.0; 105.6 (d, J=24 Hz); 112.5 (d, J=26 Hz); 114.4 (d, J=9 Hz); 118.6; 118.7; 126.0; 126.4; 126.5; 127.2; 130.5 (d, J=10 Hz); 131.1; 139.4; 158.6 (d, J=237 Hz).

### Example 63

### 1-(3-(((4-(Dimethylamino)-4-phenylcyclohexyl)methoxy)methyl)-5-fluoro-1H-indol-1-yl)ethanone (one of two possible diastereomers)

A suspension of benzyltrimethyl ammonium fluoride monohydrate (108 mg, 0.58 mmol) and molecular sieve 4 Å (2.0 g) in anhydrous tetrahydrofuran (30 ml) was stirred for 1 h at room temperature. A solution of Example 16 (100 mg, 0.18 mmol) in anhydrous tetrahydrofuran (30 ml) was then added to this suspension. The mixture was stirred for 4 h at 40° C. and then overnight at room temperature. The suspension was filtered, the filter residue washed with tetrahydrofuran (2×20 ml) and the filtrate concentrated to small volume under vacuum. The crude product (110 mg) was purified by flash chromatography (18 g, 18×1.6 cm) with dichloromethane/methanol (95:5).

Yield: 37 mg (47%), yellowish oil

¹H-NMR (CDCl₃): 1.45-1.82 (m, 7H); 2.04 (s, 6H); 2.45-2.58 (m, 2H); 2.62 (s, 3H); 3.47 (d, 2H, J=6.7 Hz); (d, 2H, J=0.8 Hz); 7.08 (dt, 1H, J=2.6, 9.1 Hz); 7.22-7.40 (m, 6H); 7.43 (s, 1H); 8.44 (dd, 1H, J=4.7, 9.1 Hz).

¹³C-NMR (CDCl₃): 23.5; 24.7; 29.7; 32.0; 36.8; 37.6; 58.3; 60.2; 64.7; 75.5; 105.4 (d, J=24 Hz); 113.0 (d, J=25 Hz); 117.7 (d, J=9 Hz); 119.8; 124.9; 126.4; 126.9; 127.3; 130.7 (d, J=10 Hz); 132.4; 159.7 (d, J=241 Hz); 168.1.

### Example 64

### 4-(((2-tert-Butyl-5-fluoro-1H-indol-3-yl)methoxy)methyl)-N,N-dimethyl-1-phenylcyclohexanamine (one of two possible diastereomers)

A mixture of Ain-13 (522 mg, 1.60 mmol), 4-fluoro-2-iodoaniline (Ian-04) (453 mg, 1.90 mmol), [1,3-bis-(2,6-diisopropylphenyl)imidazol-2-ylidene]-3-chloropyridyl palladium(II) chloride (PEPPSI, 109 mg, 0.16 mmol) and sodium carbonate (848 mg, 8.0 mmol) was evacuated for 30 min (oil pump). The mixture was then flushed with argon and absolute N,N-dimethyl formamide (5 ml, previously flushed for 1 h with argon) was added with a syringe via a Schlenk attachment. The reaction mixture was stirred for 18 h at 10° C., during the course of which the solution turned a dark brown colour. The reaction solution was then concentrated to small volume under vacuum, the residue was taken up repeatedly in toluene (3×30 ml) and concentrated to small volume again each time, then divided between water and ethyl acetate (50 ml each) and the phases were separated. The aqueous phase was extracted with ethyl acetate (2×50 ml), the combined organic phases were washed with 1 M sodium thiosulfate solution and saturated sodium chloride solution (50 ml each), dried with sodium sulfate and concentrated to small volume under vacuum. The crude product (948 mg) was purified by flash chromatography (38 g, 20×2.5 cm) with chloroform/methanol (1:5).

Yield: 421 mg (60%)

Melting point: 51-53° C.

¹H-NMR (DMSO-d₆): 1.19-1.68 (m, 7H); 1.44 (s, 9H); 1.89 (s, 6H); 2.54-2.64 (m, 2H); 3.29-3.31 (m, 2H); 4.64 (s, 2H); 6.84 (dt, 1H, J=2.5, 9.6 Hz); 7.23 (dd, 2H, J=2.5, 10.2 Hz); 7.27 (d, 1H, J=4.7 Hz); 7.29-7.37 (m, 4H); 10.79 (s, 1H).

¹³C-NMR (DMSO-d₆): 24.5; 30.3; 32.1; 33.2; 37.0; 37.4; 58.5; 63.2; 74.5; 102.2 (d, 1C, J=23 Hz); 106.6; 108.1 (d, 1C, J=26 Hz); 111.6 (d, 1C, J=0 Hz); 126.1; 126.5; 127.2; 129.8 (d, 1C, J=10 Hz); 130.7; 139.4; 147.5; 156.9 (d, 1C, J=231 Hz). Other C signals were unable to be identified.

### Example 65

### 3-(((4-(Azetidin-1-yl)-4-phenylcyclohexyl)methoxy)methyl)-2-(triethylsilyl)-1H-pyrrolo[2,3-b]pyridine (one of two possible diastereomers)

A mixture of Ain-10 (473 mg, 1.19 mmol), 2-amino-3-iodopyridine (Ian-05) (308 mg, 1.4 mmol), [1,3-bis-(2,6-diisopropylphenyl)imidazol-2-ylidene]-(3-chloropyridyl)palladium(II) chloride (PEPPSI, 162 mg, 0.24 mmol) and sodium carbonate (630 mg, 5.9 mmol) in oxygen-free and anhydrous N,N-dimethyl formamide (10 ml) was stirred for 18 h at 100° C. The solvent was then removed under vacuum and the residue divided between water and diethyl ether (20 ml each). The aqueous phase was extracted with diethyl ether (3×10 ml) and the organic phase was washed with water and 1 M sodium thiosulfate solution (20 ml each). The combined organic phases were dried with sodium sulfate and concentrated to small volume under vacuum. The crude product (700 mg) was purified by flash chromatography (18 g, 20×2.0 cm) with ethyl acetate/cyclohexane (1:2).

Yield: 346 mg (59%), yellowish oil

¹H-NMR (DMSO-d₆): 0.90-0.97 (m, 15H); 1.20-1.32 (m, 2H); 1.42-1.54 (m, 5H); 1.59 (quin, 2H, J=6.8 Hz); 2.17 (d, 2H, J=13.1 Hz); 2.81 (t, 4H, J=6.8 Hz); 3.33 (d, 2H, J=5.1 Hz); 4.62 (s, 2H); 7.05 (dd, 1H; J=7.8, 4.6 Hz); 7.22-7.31 (m, 3H); 7.34-7.40 (m, 2H); 7.99 (dd, 1H, J=7.8, 1.3 Hz); 8.23 (dd, 1H, J=4.6, 1.6 Hz); 11.28 (s, 1H).

¹³C-NMR (DMSO-d₆): 3.0; 7.2; 15.5; 24.9; 26.3; 30.4; 36.8; 45.8; 57.2; 64.4; 74.8; 115.0; 120.4; 126.2; 126.6; 126.7; 127.4; 134.4; 140.2; 143.0; 150.7.

### Example 66

### 3-(((4-(Azetidin-1-yl)-4-phenylcyclohexyl)methoxy)methyl)-1H-pyrrolo[2,3-b]pyridine (one of two possible diastereomers)

Activated molecular sieve 4 Å (2.00 g) was added to a suspension of benzyltrimethyl ammonium fluoride monohydrate (232 mg, 1.37 mmol) in anhydrous tetrahydrofuran (20 ml) and the mixture was stirred for 1 h at room temperature. A solution of Example 65 (224 mg, 0.45 mmol) in anhydrous tetrahydrofuran (20 ml) was then added to this suspension and the mixture was refluxed whilst stirring for 2 h. The reaction mixture was then filtered, the filter residue washed with tetrahydrofuran and the filtrate concentrated to small volume under vacuum. The residue (220 mg) was purified by flash chromatography (10 g, 20×1.5 cm) with ethyl acetate/methanol (9:2).

Yield: 59 mg (35%)

¹H-NMR (CDCl₃): 1.40-1.75 (m, 9H); 2.12-2.20 (m, 2H); 2.96 (br s, 4H); 3.44 (d, 2H, J=6.3 Hz); 4.72 (s, 2H); 7.12 (dd, 1H, J=7.8, 4.8 Hz); 7.24-7.43 (m, 6H); 8.07 (dd, 1H, J=7.8, 1.5 Hz); 8.34 (dd, 1H, J=4.7, 1.5 Hz); 10.32 (s, 1H).

¹³C-NMR (CDCl₃): 16.1; 25.0; 30.4; 36.8; 46.5; 65.1; 65.3; 75.0; 112.3; 115.8; 119.9; 124.0; 126.5; 127.1; 127.6; 128.0; 128.2; 143.0; 149.2.

### Example 67

### 4-(((2-tert-Butyl-1H-pyrrolo[2,3-b]pyridin-3-yl)methoxy)methyl)-N,N-dimethyl-1-phenylcyclohexanamine (one of two possible diastereomers)

A mixture of Ain-13 (347 mg, 1.06 mmol), 2-amino-3-iodopyridine (Ian-05) (280 mg, 1.27 mmol), [1,3-bis-(2,6-diisopropylphenyl)imidazol-2-ylidene]-(3-chloropyridyl) palladium(II) chloride (PEPPSI, 72 mg, 0.11 mmol) and sodium carbonate (562 mg, 5.3 mmol) was evacuated for 30 min (oil pump). The mixture was then flushed with argon and absolute N,N-dimethyl formamide (5 ml, previously flushed for 1 h with argon) was added with a syringe via a Schlenk attachment. The reaction mixture was stirred for 18 h at 100° C., during the course of which it turned a dark brown colour. The reaction solution was then concentrated to small volume under vacuum, the residue was taken up repeatedly in toluene (3×30 ml) and concentrated to small volume again each time, then divided between water and ethyl acetate (50 ml each) and the phases were separated. The aqueous phase was extracted with ethyl acetate (2×50 ml), the combined organic phases were washed with 1 M sodium thiosulfate solution and saturated sodium chloride solution (50 ml each), dried with sodium sulfate and concentrated to small volume under vacuum. The crude product (581 mg) was purified by flash chromatography (38 g, 20×2.5 cm) with chloroform/methanol (95:5).

Yield: 77 mg (17%)

Melting point: 67-70° C.

¹H-NMR (DMSO-d₆): 1.21-1.44 (m, 6H); 1.46 (s, 9H); 1.48-1.55 (m, 1H); 1.56-1.65 (m, 1H); 1.89 (br s, 6H); 2.52-2.60 (m, 1H); 3.31-3.33 (m, 2H); 4.67 (s, 2H); 7.02 (dd, 1H, J=4.7, 7.8 Hz); 7.22 (very broad s, 1H), 7.31 (very broad s, 4H); 7.90 (dd, 1H, J=1.5, 7.8 Hz); 8.14 (dd, 1H, J=1.5, 4.7 Hz); 11.30 (s, 1H).

¹³C-NMR (DMSO-d₆): 24.5; 30.2; 32.0; 33.5; 37.0; 37.4; 58.5; 62.9; 74.5; 79.1; 104.8; 115.1; 121.6; 125.4; 126.1; 126.5; 127.2; 141.6; 146.2; 147.2.

### Example 68

### N,N-Dimethyl-1-phenyl-4-(((1-(phenylsulfonyl)-1H-indol-5-yl)methoxy)methyl)cyclohexanamine (one of two possible diastereomers)

### Stage 1: 1-Benzenesulfonyl-5-methyl-1H-indole

A 60% dispersion of sodium hydride in mineral oil (1.55 g, 38.9 mmol) was added at 0° C. within 30 min to a solution of 5-methylindole (5.0 g, 38 mmol) in anhydrous tetrahydrofuran (60 ml). The suspension formed was stirred for 1 h at room temperature, then benzenesulfonyl chloride (6.89 g, 5.0 ml, 38.9 mmol) was added dropwise and the mixture was stirred overnight at room temperature. 5% sodium hydrogen carbonate solution (200 ml) was then added to the reaction mixture and it was extracted with diethyl ether (3×80 ml). The combined organic phases were dried with sodium sulfate and concentrated to small volume under vacuum. The crude product (10.4 g) was purified by flash chromatography with ethyl acetate/cyclohexane (1:9).

Yield: 9.1 g (88%), light brown oil

¹H-NMR (DMSO-d₆): 2.34 (s, 3H); 6.76 (dd, 1H, J=0.8, 3.7 Hz); 7.13-7.18 (m, 1H); 7.37 (m, 1H); 7.53-7.71 (m, 3H); 7.74 (d, 1H, J=3.7 Hz); 7.81 (d, 1H, J=8.5 Hz); 7.91-7.96 (m, 2H).

### Stage 2: 1-Benzenesulfonyl-5-bromomethyl-1H-indole

Azoisobutyronitrile (50 mg) and N-bromosuccinimide (6.10 g, 34.3 mmol) were added to a solution of 1-benzenesulfonyl-5-methyl-1H-indole (7.77 g, 28.6 mmol) in tetrachloromethane (230 ml) whilst refluxing and the mixture was refluxed for a further 5 h. The precipitated deposit was filtered off and the filtrate was concentrated to small volume under vacuum. The residue was purified by flash chromatography (570 g, 35×6.8 cm) with ethyl acetate/cyclohexane (1:9).

Yield: 3.29 g (33%), white solid

¹H-NMR (DMSO-d₆): 4.79 (s, 2H); 6.85 (dd, 1H, J=0.7, 3.7 Hz); 7.42 (dd, 1H, J=1.8, 8.6 Hz); 7.56-7.62 (m, 2H); 7.67-7.72 (m, 2H); 7.85 (d, 1H, J=3.7 Hz); 7.89-7.95 (m, 1H); 7.97-8.00 (m, 2H).

### Stage 3: 1-Benzenesulfonyl-5-(1,4-dioxaspiro[4.5]dec-8-ylmethoxymethyl)-1H-indole

A mixture of 1-benzenesulfonyl-5-bromomethyl-1H-indole (1.70 g, 4.85 mmol), (1,4-dioxaspiro[4.5]dec-8-yl)methanol (1.25 g, 7.27 mmol), N,N,N′,N′-tetramethyl-1,8-naphthalene diamine (1.04 g, 4.85 mmol) and copper(II) acetyl acetonate (64 mg, 0.24 mmol) in p-xylene (20 ml) was stirred for 21 h at 140° C. The solvent was then removed under vacuum and the residue purified by flash chromatography (230 g, 27×5.2 cm) with ethyl acetate/cyclohexane (1:4).

Yield: 430 mg (20%), viscous yellow oil

¹H-NMR (DMSO-d₆): 1.09-1.22 (m, 2H); 1.36-1.46 (m, 2H); 1.52-1.71 (m, 5H); 3.25 (d, 2H, J=6.40 Hz); 3.82 (s, 4H); 4.48 (s, 2H); 6.84 (dd, 1H, J=0.8, 3.7 Hz); 7.29 (dd, 1H, J=1.6, 8.6 Hz); 7.52-7.54 (m, 1H); 7.55-7.61 (m, 2H); 7.65-7.71 (m, 1H); 7.79 (d, 1H, J=3.7 Hz); 7.89-7.93 (m, 1H); 7.94-7.99 (m, 2H).

### Stage 4: 4-[1-Benzenesulfonyl-1H-indol-5-ylmethoxymethyl)cyclohexanone

2 N Hydrochloric acid (500 μl) was added to a solution of 1-benzenesulfonyl-5-(1,4-dioxaspiro[4.5]dec-8-ylmethoxymethyl)-1H-indole (212 mg, 0.48 mmol) in tetrahydrofuran (2.5 ml) and the mixture was stirred for 5 h at 60° C. The reaction mixture was then adjusted to pH 8 with sodium hydrogen carbonate solution. The tetrahydrofuran was distilled off under vacuum and the aqueous solution was extracted with dichloromethane (2×10 ml). The combined organic phases were washed with sodium chloride solution, dried with sodium sulfate and concentrated to small volume under vacuum. The crude product (170 mg) was purified by flash chromatography (18 g, 20×2 cm) with ethyl acetate/cyclohexane (1:2).

Yield: 120 mg (63%), brownish oil

¹H-NMR (DMSO-d₆): 1.27-1.44 (m, 2H); 1.90-2.10 (m, 3H); 2.11-2.23 (m, 2H); 2.29-2.43 (m, 2H); 3.35 (d, 2H, J=6.4 Hz); 4.52 (s, 2H); 6.84 (dd, 1H, J=0.7, 3.7 Hz); 7.30 (dd, 1H, J=8.6 Hz); 7.53-7.62 (m, 3H); 7.65-7.72 (m, 1H); 7.81 (d, 1H, J=1.5, 3.7 Hz); 7.88-8.00 (m, 3H).

### Stage 5: 4-[1-Benzenesulfonyl-1H-indol-5-ylmethoxymethyl)-1-dimethylaminocyclohexane carbonitrile

40% aqueous dimethylamine solution (420 μl, 3.32 mmol) followed by a solution of 4-[1-benzenesulfonyl-1H-indol-5-ylmethoxymethyl)cyclohexanone (330 mg, 0.83 mmol) in methanol (1 ml) and tetrahydrofuran (2.5 ml) were added to a solution of 4 N hydrochloric acid (210 μl, 0.84 mmol) and methanol (0.5 ml) that had been cooled to 0° C. Potassium cyanide (130 mg, 2.0 mmol) was added to this mixture and it was stirred at room temperature for 5 h. After adding water (5 ml) it was extracted with diethyl ether (3×10 ml). The combined organic phases were dried with sodium sulfate and concentrated to small volume under vacuum.

Yield: 315 mg (84%), brown oil

¹H-NMR (DMSO-d₆): 1.00-2.11 (m, 9H); 2.19 and 2.23 (2s, 6H); 3.19-3.31 (m, 2H); 4.46-4.53 (m, 2H); 6.83-6.86 (m, 1H); 7.26-7.33 (m, 1H); 7.51-7.78 (m, 4H); 7.79-7.82 (m, 1H); 7.88-8.00 (m, 3H).

The diastereoisomer ratio was approximately 2:3.

### Stage 6: N,N-Dimethyl-1-phenyl-4-(((1-(phenylsulfonyl)-1H-indol-5-yl)methoxy)methyl)cyclohexanamine (one of two possible diastereomers)

A solution of 4-[1-benzenesulfonyl-1H-indol-5-ylmethoxymethyl)-1-dimethylaminocyclohexane carbonitrile (315 mg, 0.7 mmol) in anhydrous tetrahydrofuran (3 ml) was added dropwise under argon to an ice-cooled 2 M solution of phenyl magnesium chloride in tetrahydrofuran (1.4 ml, 2.8 mmol) and then the mixture was stirred for 3 days at room temperature. 20% ammonium chloride solution (5 ml) was then added to the reaction mixture. The phases were separated and the aqueous phase was extracted with diethyl ether (2×10 ml). The combined organic phases were washed with sodium chloride solution (20 ml), dried with sodium sulfate and concentrated to small volume under vacuum. The crude product (432 mg) was purified by flash chromatography (37 g, 20×2.5 cm) with ethyl acetate/methanol (9:1).

Yield: 89 mg (25%), yellow oil

¹H-NMR (CDCl₃): 1.44-1.77 (m, 7H); 2.01 (s, 6H); 2.48-2.57 (m, 2H); 3.41 (d, 2H, J=6.6 Hz); 4.56 (s, 2H); 6.65 (dd, 1H, J=0.7, 3.7 Hz); 7.21-7.58 (m, 11H); 7.85-7.89 (m, 2H); 7.97 (d, 1H, J=8.5 Hz).

Only one diastereoisomer was isolated.

### Example 69

### 4-(((1H-Indol-5-yl)methoxy)methyl)-N,N-dimethyl-1-phenylcyclohexanamine (one of two possible diastereomers)

2 N Sodium hydroxide solution (1 ml) was added to a solution of N,N-dimethyl-1-phenyl-4-(((1-(phenylsulfonyl)-1H-indol-5-yl)methoxy)methyl)cyclohexanamine (50 mg, 0.1 mmol) in methanol (4 ml) and the mixture was refluxed for 6.5 h whilst stirring. Methanol was then distilled off under vacuum and the aqueous residue was adjusted to pH 6-7 with acetic acid and extracted with dichloromethane (2×10 ml). The combined organic phases were dried with sodium sulfate and concentrated to small volume under vacuum. The solution of the crude product in dichloromethane/methanol was made alkaline with aqueous ammonia, concentrated to small volume under vacuum and dried. The residue was purified by flash chromatography (10 g, 17×1.5 cm) with dichloromethane/methanol (10:1).

Yield: 35 mg (96%), yellow oil

¹H-NMR (CDCl₃): 0.81-0.93 (m, 2H); 1.26 (s, 3H); 1.65-1.83 (m, 2H); 2.09 (br s, 6H); 2.46-2.57 (m, 2H); 3.36-3.49 (m, 2H); 4.63 (s, 2H); 6.52-6.55 (m, 1H); 7.19-7.42 (m, 8H); 7.61-7.63 (m, 1H); 8.26 (s, 1H).

### Example 70

### N,N-Dimethyl-1-phenyl-4-(((1-(phenylsulfonyl)-1H-indol-4-yl)methoxy)methyl)cyclohexanamine (one of two possible diastereomers)

### Stage 1: 1-Benzenesulfonyl-4-methyl-1H-indole

Pulverised sodium hydroxide (2.93 g, 73.2 mmol) and tetra-n-butylammonium hydrogen sulfate (20 mg) were added to a solution of 4-methylindole (4.80 g, 36.6 mmol) in anhydrous dichloromethane (40 ml) and the mixture was stirred for 1 h at room temperature. Benzenesulfonyl chloride (7.11 g, 5.2 ml, 40.3 mmol) was then added dropwise to the mixture at 0° C. within 20 min (vigorous reaction!) and the mixture was then stirred overnight at room temperature. Water and dichloromethane (50 ml each) were then added to the reaction mixture. The organic phase was separated off, washed with sodium chloride solution, dried with sodium sulfate and concentrated to small volume under vacuum.

Yield: 4.86 g (49%), pale yellow solid

¹H-NMR (DMSO-d₆): 2.42 (s, 3H); 6.89 (d, 1H, J=3.4 Hz); 7.05 (d, 1H, J=7.3 Hz); 7.23 (t, 1H, J=7.9 Hz); 7.57 (t, 2H, J=7.7 Hz); 7.67 (t, 1H, J=7.3 Hz); 7.76 (d, 1H, J=8.4 Hz); 7.8 (d, 1H J=3.67 Hz); 7.96 (d, 2H, J=7.8 Hz).

### Stage 2: 1-Benzenesulfonyl-4-bromomethyl-1H-indole

Azoisobutyronitrile (50 mg) and N-bromosuccinimide (3.10 g, 17.3 mmol) were added to a solution of 1-benzenesulfonyl-4-methyl-1H-indole (3.90 g, 14.4 mmol) in carbon tetrachloride (120 ml) whilst refluxing and the mixture was refluxed for a further 4 h. The precipitated deposit was filtered off, the filtrate was concentrated to small volume under vacuum and the residue was purified by flash chromatography with ethyl acetate/cyclohexane (1:9).

Yield: 4.20 g (83%), white solid

¹H-NMR (DMSO-d₆): 4.95 (s, 2H); 7.05 (dd, 1H, J=0.8, 3.7 Hz); 7.30-7.35 (m, 2H); 7.55-7.66 (m, 3H); 7.89-8.04 (m, 4H).

### Stage 3: 1-Benzenesulfonyl-4-(1,4-dioxaspiro[4.5]dec-8-ylmethoxymethyl)-1H-indole

A mixture of 1-benzenesulfonyl-4-bromomethyl-1H-indole (4.00 g, 11.4 mmol), (1,4-dioxaspiro[4.5]dec-8-yl)methanol (2.90 g, 17.1 mmol), ethyl diisopropylamine (1.47 g, 1.9 ml, 11.4 mmol) and copper(II) acetyl acetonate (149 mg, 0.57 mmol) in p-xylene (70 ml) was stirred in a Teflon pressure vessel for 24 h at 140° C. The solvent was then removed under vacuum. The residue was taken up in dichloromethane (50 ml) and the solution washed with 0.05 N hydrochloric acid and water (50 ml each). The organic phase was dried with sodium sulfate and concentrated to small volume. The crude product (5.8 g) was purified by flash chromatography (400 g, 21×7.0 cm) with ethyl acetate/cyclohexane (1:4).

Yield: 912 mg (18%), white waxy solid

¹H-NMR (DMSO-d₆): 1.10-1.20 (m, 2H); 1.35-1.45 (m, 2H); 1.57-1.67 (m, 5H); 3.26 (d, 2H, J=6.3 Hz); 3.82 (s, 4H); 4.64 (s, 2H); 6.89 (dd, 1H, J=0.8, 3.7 Hz); 7.19-7.22 (m, 1H); 7.29-7.34 (m, 1H); 7.56-7.61 (m, 2H); 7.66-7.71 (m, 1H); 7.82 (d, 1H, J=3.7 Hz); 7.88 (d, 1H, J=8.3 Hz); 7.95-7.99 (m, 2H).

### Stage 4: 4-[1-Benzenesulfonyl-1H-indol-4-ylmethoxymethyl)cyclohexanone

2 N Hydrochloric acid (5 ml) was added to a solution of 1-benzenesulfonyl-4-(1,4-dioxaspiro[4.5]dec-8-ylmethoxymethyl)-1H-indole (920 mg, 2.1 mmol) in tetrahydrofuran (25 ml) and the mixture was stirred for 5 h at 70° C. The reaction mixture was then adjusted to pH 8 with sodium hydrogen carbonate solution. The phases were separated and the aqueous phase was extracted with dichloromethane (2×20 ml). The combined organic phases were dried with sodium sulfate and concentrated to small volume under vacuum.

Yield: 776 mg (92%), yellow oil

¹H-NMR (DMSO-d₆): 1.23-1.40 (m, 2H); 1.87-2.08 (m, 3H); 2.09-2.20 (m, 2H); 2.34 (dt, 2H, J=5.9, 13.7 Hz); 3.35 (d, 2H, J=6.4 Hz); 4.68 (s, 2H); 6.91 (dd, 1H, J=0.8, 3.72 Hz); 7.20-7.25 (m, 1H); 7.29-7.36 (m, 1H); 7.54-7.72 (m, 3H); 7.84 (d, 1H, J=3.72 Hz); 7.89 (d, 1H, J=8.2 Hz,); 7.95-8.01 (m, 2H).

### Stage 5: 4-[1-Benzenesulfonyl-1H-indol-4-ylmethoxymethyl)-1-dimethylaminocyclohexane carbonitrile

A solution of 4 N hydrochloric acid (0.87 ml, 3.48 mmol), followed by potassium cyanide (227 mg, 3.48 mmol) and then a solution of 4-[1-benzenesulfonyl-1H-indol-4-ylmethoxymethyl)cyclohexanone (578 mg, 1.45 mmol) in methanol/tetrahydrofuran (2 ml/1 ml) were added to a 40% aqueous dimethylamine solution (1.1 ml, 8.7 mmol) and methanol (0.3 ml) cooled to 0° C. This mixture was stirred overnight at room temperature. After adding water (3 ml) it was extracted with diethyl ether (2×10 ml). The combined organic phases were dried with sodium sulfate and concentrated to small volume under vacuum.

Yield: 630 mg (96%), viscous yellow oil

¹H-NMR (DMSO-d₆): 1.01-1.37 (m, 4H); 1.39-1.83 (m, 3H); 1.97-2.11 and 2.11-2.21 (2 m, 2H); 2.18 and 2.23 (2 s, 6H); 3.25 (d, 0.33H, J=6.5 Hz); 3.28 (d, 1.67H, J=6.3 Hz); 4.64 and 4.66 (2 s, 2H); 6.88-6.92 (m, 1H); 7.18-7.24 (m, 1H); 7.28-7.35 (m, 1H); 7.55-7.63 (m, 2H); 7.65-7.72 (m, 1H); 7.83 (d, 1H, J=3.7 Hz); 7.88 (d, 1H, J=8.25 Hz); 7.95-8.01 (m, 2H).

The diastereoisomer ratio is approximately 1:5.

### Stage 6: N,N-Dimethyl-1-phenyl-4-(((1-(phenylsulfonyl)-1H-indol-4-yl)methoxy)methyl)cyclohexanamine (one of two possible diastereomers)

A solution of 4-[1-benzenesulfonyl-1H-indol-4-ylmethoxymethyl)-1-dimethylaminocyclohexane carbonitrile (630 mg, 1.4 mmol) in anhydrous tetrahydrofuran (4 ml) was added dropwise under argon to an ice-cooled 2 M solution of phenyl magnesium chloride in tetrahydrofuran (2.8 ml, 5.58 mmol) and then the mixture was stirred overnight at room temperature. 20% ammonium chloride solution (5 ml) was then added to the reaction mixture. The phases were separated and the aqueous phase was extracted with diethyl ether (2×10 ml). The combined organic phases were washed with sodium chloride solution (20 ml), dried with sodium sulfate and concentrated to small volume under vacuum. The crude product (554 mg) was purified by flash chromatography (30 g, 18×2.5 cm), first with ethyl acetate and then again (20 g, 22×2.0 cm) with ethyl acetate/cyclohexane (2:1).

Yield: 296 mg (42%), colourless oil

¹H-NMR (DMSO-d₆): 1.26-1.46 (m, 4H); 1.45-1.56 (m, 2H); 1.54-1.68 (m, 1H); 1.89 (s, 6H); 2.51-2.58 (m, 2H); 3.30-3.32 (m, 2H); 4.66 (s, 2H); 6.93 (dd, 1H, J=0.8, 3.7 Hz); 7.20-7.25 (m, 2H); 7.27-7.36 (m, 5H); 7.54-7.61 (m, 2H); 7.65-7.70 (m, 1H); 7.84 (d, 1H, J=3.7 Hz); 7.89 (d, 1H, J=8.3 Hz); 7.96-8.00 (m, 2H).

¹³C-NMR (DMSO-d₆): 24.4 (2C); 32.0 (2C); 36.9; 37.4 (2C); 58.5; 70.1; 75.3; 108.0; 112.3; 122.4; 124.5; 126.1; 126.5 (2C); 126.6 (2C); 126.7; 127.2 (2C); 129.1; 129.8 (2C); 131.7; 134.1; 134.5; 137.0; 139.4.

The product is a uniform diastereoisomer.

### Example 71

### 4-(((1H-Indol-4-yl)methoxy)methyl)-N,N-dimethyl-1-phenylcyclohexanamine (one of two possible diastereomers)

2 N Sodium hydroxide solution (2 ml) was added to a solution of N,N-dimethyl-1-phenyl-4-(((1-(phenylsulfonyl)-1H-indol-4-yl)methoxy)methyl)cyclohexanamine (230 mg, 0.46 mmol) in methanol (20 ml) and the mixture was refluxed for 4.5 h whilst stirring. The reaction mixture was then adjusted to pH 5-6 with 2N hydrochloric acid and methanol was distilled off under vacuum. The aqueous solution was adjusted to pH 8-9 with sodium hydrogen carbonate and extracted with dichloromethane (2×15 ml). The combined organic phases were dried with sodium sulfate and concentrated to small volume under vacuum. The residue (159 mg) was purified by flash chromatography (10 g, 16×1.5 cm) with dichloromethane/methanol (9:1).

Yield: 87 mg (52%), colourless oil

¹H-NMR (CDCl₃): 1.48-1.84 (m, 7H); 2.05 (s, 6H); 2.46-2.56 (m, 2H); 3.48 (d, 2H, J=6.8 Hz); 4.85 (s, 2H); 6.68-6.71 (m, 1H); 7.10-7.14 (m, 1H); 7.15-7.18 (m, 1H); 7.19-7.22 (m, 1H); 7.30-7.39 (m, 6H); 8.34 (br s, 1H).

¹C-NMR (CDCl₃): 24.7; 32.0 (very broad); 37.0 (very broad); 37.8; 59.7 (very broad); 71.9; 75.3 (very broad); 101.2; 110.6; 119.1; 121.7; 124.0; 126.4; 126.7; 127.0; 127.5; 128.4; 130.5; 135.9.

LC-MS (method 7): m/z: [M+H]⁺=363.3, Rₜ 2.9 min.

### Example 72

### N,N-Dimethyl-1-phenyl-4-(((2-(triethylsilyl)-5-(trifluoromethoxy)-1H-indol-3-yl)methoxy)methyl)cyclohexanamine (one of two possible diastereomers)

A mixture of Ain-1 (1.10 g, 2.8 mmol), 4-trifluoromethoxy-2-iodoaniline (Ian-12) (1.13 g, 3.7 mmol), [1,3-bis-(2,6-diisopropylphenyl)imidazol-2-ylidene]-(3-chloropyridyl)palladium(II) chloride (PEPPSI, 380 mg, 0.56 mmol) and sodium carbonate (1.48 g, 14 mmol) in anhydrous N,N-dimethyl formamide (15 ml) was stirred for 18 h at 100° C. The solvent was removed under vacuum and the residue divided between water and diethyl ether (60 ml each). The aqueous phase was extracted with diethyl ether (3×20 ml). The combined organic phases were washed with water and sodium thiosulfate solution (30 ml each), dried with sodium sulfate and concentrated to small volume under vacuum. The crude product (2.70 g) was purified by flash chromatography (100 g, 20×4.0 cm) with ethyl acetate/cyclohexane (1:6).

Yield: 1.33 g (85%), brownish oil

¹H-NMR (DMSO-d₆): 0.86-0.99 (m, 15H); 1.28-1.65 (m, 9H); 1.87 (s, 6H); 2.51-2.58 (m, 2H); 4.63 (s, 2H); 7.06 (d, 1H, J=8.3 Hz); 7.18-7.24 (m, 1H); 7.27-7.37 (m, 4H), 7.47 (d, 1H, J=8.7 Hz); 7.52 (s, 1H); 10.92 (s, 1H).

¹³C-NMR (DMSO-d₆): 3.1; 7.2; 24.5; 32.0; 37.0; 37.4, 58.5; 64.4; 74.7; 110.8; 112.3; 115.1; 120.4 (q, J=254 Hz); 121.9; 126.1; 126.5; 127.2; 128.4; 136.2; 137.1; 139.4; 141.5. m/z: [M+H]⁺=561.4

### Example 73

### N,N-Dimethyl-1-phenyl-4-(((5-(trifluoromethoxy)-1H-indol-3-yl)methoxy)methyl)cyclohexanamine (one of two possible diastereomers)

Activated molecular sieve 4 Å (4.00 g) was added to a suspension of benzyltrimethyl ammonium fluoride monohydrate (966 mg, 5.7 mmol) in anhydrous tetrahydrofuran (60 ml) and the mixture was stirred for 1 h at room temperature. A solution of Example 72 (1.08 g, 1.9 mmol) in anhydrous tetrahydrofuran (30 ml) was then added to this suspension and the mixture was refluxed whilst stirring for 1 h. The reaction mixture was then filtered, the filtrate concentrated to small volume under vacuum and the residue (1.13 g) purified by flash chromatography (100 g, 20×4.0 cm) with ethyl acetate/methanol (95:5).

Yield: 546 mg (64%), yellowish oil

¹H-NMR (DMSO-d₆): 1.28-1.64 (m, 7H); 1.89 (s, 6H); 2.54 (br d, 2H, J=14.3 Hz); 3.29 (br d, 2H, J=6.3 Hz); 4.62 (s, 2H); 7.04-7.09 (m, 1H); 7.19-7.25 (m, 1H); 7.27-7.36 (m, 4H); 7.44-7.53 (m, 3H), 11.25 (s, 1H).

¹³C-NMR (DMSO-d₆): 24.5; 32.0; 37.0; 37.4; 58.5; 64.2; 74.3; 111.0; 112.5; 112.8; 114.7; 120.4 (q, J=254 Hz); 126.1; 126.5; 126.9; 127.0; 127.2; 134.8; 139.4; 141.6 (broad). m/z: [M+H]⁺=447.3

Investigations into the Effectiveness of the Compounds According to the Invention:

### Measurement of ORL1 Binding

The cyclohexane derivatives having the general formula I were investigated in a receptor binding assay with ³H-nociceptin/orphanin FQ with membranes of recombinant CHO-ORL1 cells. These measurements were performed in accordance with the method described by Ardati et al. (Mol. Pharmacol., 51, 1997, p. 816-824). The concentration of ³H-nociceptin/orphanin FQ in these assays was 0.5 nM. The binding assays were carried out with 20 μg amounts of membrane protein per 200 μl batch in 50 mM Hepes, pH 7.4, 10 mM MgCl₂ and 1 mM EDTA. The binding to the ORL1 receptor was determined using 1 mg amounts of WGA-SPA beads (Amersham-Pharmacia, Freiburg, Germany), by incubation of the batch for one hour at room temperature and subsequent measurement in a Trilux scintillation counter (Wallac, Finland).

### Measurement of μ Binding

The receptor affinity to the human μ-opiate receptor was determined in a homogeneous batch in microtitre plates. To this end, dilution series of the substituted 4-aminocyclohexane derivative to be tested were incubated for 90 minutes at room temperature with a receptor membrane preparation (15-40 μg protein per 250 μl incubation batch) of CHO-K1 cells, which express the human μ-opiate receptor (RB-HOM receptor membrane preparation from NEN, Zaventem, Belgium), in the presence of 1 nmol/l of the radioactive ligand [³H] naloxone (NET719, NEN, Zaventem, Belgium) and 1 mg of WGA-SPA beads (wheat germ agglutinin SPA beads from Amersham/Pharmacia, Freiburg, Germany) in a total volume of 250 μl. 50 mmol/l tris-HCl supplemented with 0.05 wt. % sodium azide and 0.06 wt. % bovine serum albumin were used as the incubation buffer. In order to determine the non-specific binding, 25 μmol/l of naloxone were also added. At the end of the ninety-minute incubation period the microtitre plates were centrifuged for 20 minutes at 1000 g and the radioactivity was measured in a β counter (Microbeta-Trilux, PerkinElmer Wallac, Freiburg, Germany). The percentage displacement of the radioactive ligand from its binding to the human μ-opiate receptor was determined at a test substance concentration of 1 μmol/l and stated as the percentage inhibition (% inhibition) of the specific binding. In some cases the percentage displacement due to differing concentrations of the compounds having the general formula I to be tested was used to calculate the IC₅₀ inhibition concentrations which bring about a 50-percent displacement of the radioactive ligand. Kᵢ values for the test substances are obtained by extrapolation using the Cheng-Prusoff equation.

The compounds according to the invention bind to the ORL1 receptor and/or the μ-receptor.

Chung Model: Mononeuropathic Pain after Spinal Nerve Ligation
Animals: Male Sprague Dawley rats (140-160 g), from a commercial breeder (Janvier, Genest St. Isle, France), were kept in a 12:12 h light/dark cycle. The animals were provided with free access to food and tap water. An interval of one week was allowed between delivery of the animals and the operation. Following the operation the animals were tested several times over a period of 4 to 5 weeks, allowing an elution time of at least one week.
Description of the model: Under pentobarbital narcosis (Narcoren®, 60 mg/kg i.p., Merial GmbH, Hallbergmoos, Germany) the left spinal nerves L5 and L6 were exposed by removing a piece of the paravertebral muscle and a part of the left spinal process of the L5 lumbar vertebral body. The spinal nerves L5 and L6 were carefully isolated and tightly bound (NC silk black, USP 5/0, metric 1, Braun Melsungen AG, Melsungen, Germany) (Kim and Chung 1992). Following ligation the muscles and neighbouring tissue were sutured and the wounds closed with metal staples.

After a recovery period of one week the animals were placed in cages with a wire floor to measure the mechanical allodynia. The withdrawal threshold on the ipsilateral and contralateral rear paws was determined using an electronic von Frey filament (Somedic AB, Malmö, Sweden). The median of five consecutive stimulations was calculated for each measuring time point. The animals were tested 30 minutes before and at various times after administration of the test substance or vehicle solution. The data was calculated as the % maximum possible effect (% MPE) from the pre-tests on individual animals (=0% MPE) and the test values for an independent sham control group (=100% MPE). As an alternative the withdrawal thresholds were plotted in grams.

Statistical analysis: ED₅₀ values and 95% confidence intervals were determined by semilogarithmic regression analysis at the time of maximum effect. The data was analysed by means of a variance analysis with repeat measurements and a Bonterroni post-hoc analysis. The group size was usually n=10.
References: Kim, S. H. and Chung, J. M., An experimental model for peripheral neuropathy produced by segmental spinal nerve ligation in the rat, Pain, 50 (1992) 355-363.

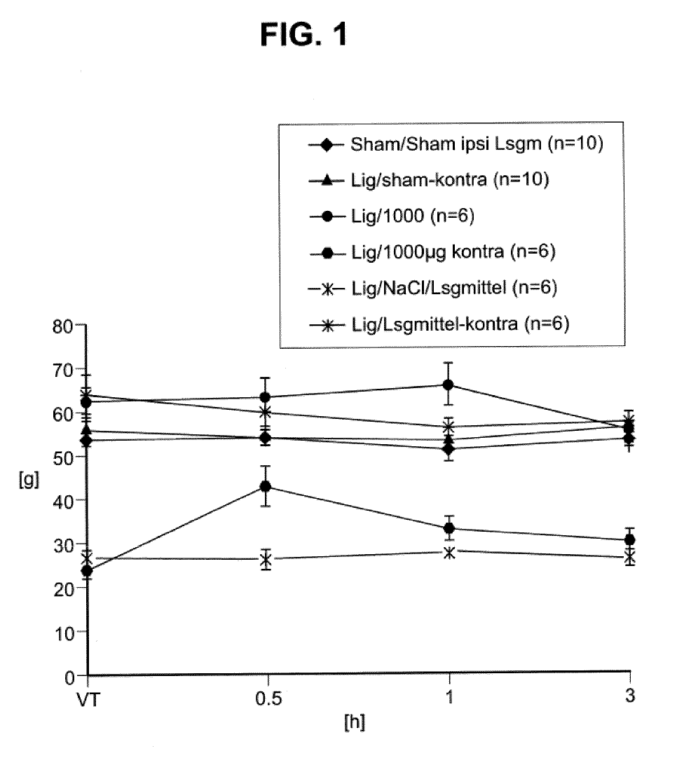

Analysis of Example 10 in the Chung model (FIG. 1) shows that the compound acts primarily on the ipsilateral side (i.e. in neuropathic pain). By contrast, only a very small and statistically insignificant effect is observed on the undamaged, contralateral side. The test shows that the compound has very good effectiveness in neuropathic pain within an animal, since the allodynia condition (visible from the fall in the withdrawal threshold from approx. 55 g to 25 g) is almost completely relieved (visible from the rise in the withdrawal threshold to over 40 g). The absence of an effect on the contralateral side indicates that in the tested dose the compound has no anti-nociceptive—in other words general pain-suppressing—effect per se. It can be concluded from this that the compound relieves the pathological allodynia condition in the neuropathic situation without having a directly analgesic effect. This means that a lower dose can be administered for the treatment of neuropathic pain than would be necessary for the treatment of acute pain.

### Parenteral Solution of a Substituted 4-Aminocyclohexane Derivative According to the Invention

38 g of one of the substituted 4-aminocyclohexane derivatives according to the invention, in this case Example 3, are dissolved in 1 l of water for injection at room temperature and then adjusted to isotonic conditions by the addition of anhydrous glucose for injection.

What is claimed is:

1. A substituted 4-aminocyclohexane derivative compound having the formula I: 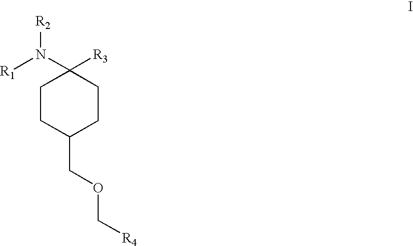
wherein
R¹ and R² independently denote C₁₋₃ alkyl or H, or R¹ and R² together with the N atom to which they are bonded form a ring with R¹ and R² together denoting —(CH₂)₃— or —(CH₂)₄—;
R³ optionally denotes aryl or heteroaryl bound by a C₁₋₃ alkyl chain, each of which aryl, heteroaryl or alkyl chain is unsubstituted or mono- or polysubstituted; or denotes C₁₋₆ alkyl, which is unsubstituted or mono- or polysubstituted;
R⁴ denotes indolyl, pyrrolo[2,3-b]pyridinyl, pyrrolo[2,3-c]pyridinyl, pyrrolo[3,2-c]pyridinyl or pyrrolo[3,2-b]pyridinyl, each of which is unsubstituted or mono- or polysubstituted;
said compound optionally being in the form of a racemic mixture, or in the form of individual enantiomers or diastereomers, or in the form of mixtures of enantiomers and/or diastereomers in any mixing ratio;
or a base and/or salt of said compound or one of said forms with a physiologically compatible acid or cation.

2. Substituted 4-aminocyclohexane derivative according to claim 1, wherein R⁴ is bound to the formula I by one of its C atoms.

3. Substituted 4-aminocyclohexane derivative according to claim 2, wherein R⁴ is bound to the formula I by its C2 or C3 atom in the pyrrolyl ring.

4. Substituted 4-aminocyclohexane derivative according to claim 2, wherein R⁴ is bound to the formula I by its C4, C5, C6 or C7 atom, it present, in the phenyl or pyridinyl ring.

5. Substituted 4-aminocyclohexane derivative according to claim 1, wherein R¹ and R² independently denote methyl or H, or R¹ and R² form a ring with inclusion of the N atom and denote —(CH₂)₃— or —(CH₂)₄—.

6. Substituted 4-aminocyclohexane derivative according to claim 1, wherein R³ stands for phenyl, benzyl or phenethyl, each of which is unsubstituted or mono- or polysubstituted on the ring; C₁₋₆ alkyl, which is unsubstituted or mono- or polysubstituted; or pyridyl, thienyl, thiazolyl, imidazolyl, 1,2,4-triazolyl or benzimidazolyl, each of which is unsubstituted or mono- or polysubstituted.

7. Substituted 4-aminocyclohexane derivative according to claim 6, wherein R³ stands for phenyl, which is unsubstituted or monosubstituted with F, Cl, CN, CH₃; thienyl; or n-butyl, which is unsubstituted or mono- or polysubstituted with OCH₃, OH or OC₂H₅.

8. Substituted 4-aminocyclohexane derivative according to claim 1, wherein R⁴ stands for 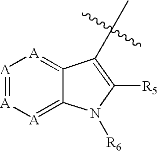
wherein
each A independently stands for N or CR⁷⁻¹⁰, wherein at most one A denotes N;
R⁵, R⁷, R⁸, R⁹ and R¹⁰ independently stand for H, F, Cl, Br, CN, CH₃, C₂H₅, NH₂, tert-butyl, Si(ethyl)₃, Si(methyl)₂(tert-butyl), SO₂CH₃, C(O)CH₃, NO₂, SH, CF₃, OH, OCH₃, OC₂H₅ or N(CH₃)₂, and
R⁶ stands for H, CH₃ or C(O)CH₃.

9. Substituted 4-aminocyclohexane derivative according to claim 8, wherein R⁴ denotes one of the structures L, M or N 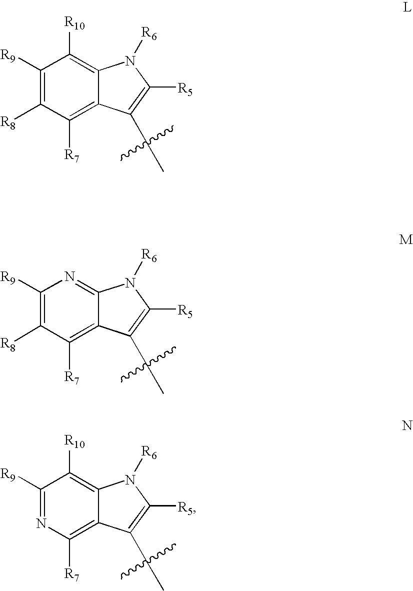
wherein the radicals
R⁵, R⁷, R⁸, R⁹ and R¹⁰ independently stand for H, F, Cl, Br, CN, CH₃, C₂H₅, NH₂, tert-butyl, Si(ethyl)₃, Si(methyl)₂(tert-butyl), SO₂CH₃, C(O)CH₃, NO₂, SH, CF₃, OH, OCH₃, OC₂H₅ or N(CH₃)₂, and
R⁶ stands for H, CH₃ or C(O)CH₃.

10. Substituted 4-aminocyclohexane derivative according to claim 1, which is selected from the group consisting of:
1 [1-Phenyl-4-(2-triethylsilanyl-1H-indol-3-ylmethoxymethyl)cyclohexyl]dimethylamine;
2 [1-Phenyl-4-(1H-indol-3-ylmethoxymethyl)cyclohexyl]dimethylamine;
3 1-Phenyl-4-(((2-(tert-butyldimethylsilyl)-5-cyano-1H-indol-3-yl)methoxy)methyl)-N,N-dimethylcyclohexanamine;
4 1-Phenyl-4-(((5-cyano-2-(triethylsilyl)-1H-indol-3-yl)methoxy)methyl)-N,N-dimethylcyclohexanamine;
5 1-Phenyl-4-(((5-cyano-1H-indol-3-yl)methoxy)methyl)-N,N-dimethylcyclohexanamine;
6 1-Phenyl-4-(((2-(tert-butyldimethylsilyl)-5-trifluoromethyl-1H-indol-3-yl)methoxy)methyl)-N,N-dimethylcyclohexanamine;
7 1-Phenyl-4-(((5-trifluoromethyl-2-(triethylsilyl)-1H-indol-3-yl)methoxy)methyl)-N,N-dimethylcyclohexanamine;
8 1-Phenyl-4-(((5-trifluoromethyl-1H-indol-3-yl)methoxy)methyl)-N,N-dimethylcyclohexanamine;
9 1-Phenyl-4-(((5-fluoro-2-(triethylsilyl)-1H-indol-3-yl)methoxy)methyl)-N,N-dimethylcyclohexanamine;
10 1-Phenyl-4-(((5-fluoro-1H-indol-3-yl)methoxy)methyl)-N,N-dimethylcyclohexanamine;
11 1-Phenyl-N,N-dimethyl-4-(((2-(triethylsilyl)-1H-pyrrolo[2,3-b]pyridin-3-yl)methoxy)methyl)cyclohexanamine;
12 4-(((1H-Pyrrolo[2,3-b]pyridin-3-yl)methoxy)methyl)-1-phenyl-N,N-dimethylcyclohexanamine;
13 1-Phenyl-4-(((2-(tert-butyldimethylsilyl)-1H-pyrrolo[3,2-c]pyridin-3-yl)methoxy)methyl)-N,N-dimethylcyclohexanamine;
14 1-Phenyl-N,N-dimethyl-4-(((2-(triethylsilyl)-1H-pyrrolo[3,2-c]pyridin-3-yl)methoxy)methyl)cyclohexanamine;
15 4-(((1H-Pyrrolo[3,2-c]pyridin-3-yl)methoxy)methyl)-1-phenyl-N,N-dimethylcyclohexanamine;
16 1-(3-(((4-(Dimethylamino)-4-phenylcyclohexyl)methoxy)methyl)-5-fluoro-2-(triethylsilyl)-1H-indol-1-yl)ethanone;
17 4-(((5-Fluoro-1-(methylsulfonyl)-2-(triethylsilyl)-1H-indol-3-yl)methoxy)methyl)-N,N-dimethyl-1-phenylcyclohexanamine;
18 1-Phenyl-4-[2-(tert-butyldimethylsilanyl)-1H-indol-3-ylmethoxymethyl]cyclohexyldimethylamine;
19 1-Phenyl-4-(((2-(tert-butyldimethylsilyl)-5-fluoro-1H-indol-3-yl)methoxy)methyl)-N,N-dimethylcyclohexanamine;
20 1-Phenyl-4-(((2-(tert-butyldimethylsilyl)-1H-pyrrolo[2,3-b]pyridin-3-yl)methoxy)methyl)-N,N-dimethylcyclohexanamine;
21 4-(((5-Fluoro-3-methyl-1H-indol-2-yl)methoxy)methyl)-N,N-dimethyl-1-phenylcyclohexanamine;
22 {1-Benzyl-4-[2-(tert-butyldimethylsilanyl)-1H-indol-3-ylmethoxymethyl]cyclohexyl}dimethylamine;
23 [1-Benzyl-4-(2-triethylsilanyl-1H-indol-3-ylmethoxymethyl)cyclohexyl]dimethylamine;
24 [1-Benzyl-4-(1H-indol-3-ylmethoxymethyl)cyclohexyl]dimethylamine;
25 1-Benzyl-4-(((5-fluoro-2-(triethylsilyl)-1H-indol-3-yl)methoxy)methyl)-N,N-dimethylcyclohexanamine;
26 1-Benzyl-4-(((2-(tert-butyldimethylsilyl)-5-fluoro-1H-indol-3-yl)methoxy)methyl)-N,N-dimethylcyclohexanamine;
27 1-Benzyl-4-(((5-fluoro-1H-indol-3-yl)methoxy)methyl)-N,N-dimethylcyclohexanamine;
28 1-Benzyl-N,N-dimethyl-4-(((2-(triethylsilyl)-1H-pyrrolo[2,3-b]pyridin-3-yl)methoxy)methyl)cyclohexanamine;
29 1-Benzyl-4-(((2-(tert-butyldimethylsilyl)-1H-pyrrolo[2,3-b]pyridin-3-yl)methoxy)methyl)-N,N-dimethylcyclohexanamine;
30 4-(((1H-Pyrrolo[2,3-b]pyridin-3-yl)methoxy)methyl)-1-benzyl-N,N-dimethylcyclohexanamine;
31 1-Benzyl-N,N-dimethyl-4-(((2-(triethylsilyl)-1H-pyrrolo[3,2-c]pyridin-3-yl)methoxy)methyl)cyclohexanamine;
32 1-Benzyl-4-(((2-(tert-butyldimethylsilyl)-1H-pyrrolo[3,2-c]pyridin-3-yl)methoxy)methyl)-N,N-dimethylcyclohexanamine;
33 4-(((1H-Pyrrolo[3,2-c]pyridin-3-yl)methoxy)methyl)-1-benzyl-N,N-dimethylcyclohexanamine;
34 1-Benzyl-N,N-dimethyl-4-(((2-(triethylsilyl)-1H-indol-3-yl)methoxy)methyl)cyclohexanamine;
35 1-Benzyl-4-(((2-(tert-butyldimethylsilyl)-1H-indol-3-yl)methoxy)methyl)-N,N-dimethylcyclohexanamine;
36 4-(((1H-Indol-3-yl)methoxy)methyl)-1-benzyl-N,N-dimethylcyclohexanamine;
37 1-Benzyl-4-(((5-fluoro-2-(triethylsilyl)-1H-indol-3-yl)methoxy)methyl)-N,N-dimethylcyclohexanamine;
38 1-Benzyl-4-(((2-(tert-butyldimethylsilyl)-5-fluoro-1H-indol-3-yl)methoxy)methyl)-N,N-dimethylcyclohexanamine;
39 1-Benzyl-4-(((5-fluoro-1H-indol-3-yl)methoxy)methyl)-N,N-dimethylcyclohexanamine;
40 1-Benzyl-N,N-dimethyl-4-(((2-(triethylsilyl)-1H-pyrrolo[2,3-b]pyridin-3-yl)methoxy)methyl)cyclohexanamine hydrochloride;
41 4-(((1H-Pyrrolo[2,3-b]pyridin-3-yl)methoxy)methyl)-1-benzyl-N,N-dimethylcyclohexanamine;
42 1-Benzyl-4-(((2-(tert-butyldimethylsilyl)-1H-pyrrolo[3,2-c]pyridin-3-yl)methoxy)methyl)-N,N-dimethylcyclohexanamine;
43 1-Butyl-N,N-dimethyl-4-(((2-(triethylsilyl)-1H-indol-3-yl)methoxy)methyl)cyclohexanamine;
44 4-(((1H-Indol-3-yl)methoxy)methyl)-1-butyl-N,N-dimethylcyclohexanamine;
45 1-Butyl-4-(((5-fluoro-2-(triethylsilyl)-1H-indol-3-yl)methoxy)methyl)-N,N-dimethylcyclohexanamine;
46 1-Butyl-4-(((5-fluoro-1H-indol-3-yl)methoxy)methyl)-N,N-dimethylcyclohexanamine;
47 1-Butyl-N,N-dimethyl-4-(((2-(triethylsilyl)-1H-pyrrolo[2,3-b]pyridin-3-yl)methoxy)methyl)cyclohexanamine;
48 4-(((1H-Pyrrolo[2,3-b]pyridin-3-yl)methoxy)methyl)-1-butyl-N,N-dimethylcyclohexanamine;
49 1-Butyl-N,N-dimethyl-4-(((2-(triethylsilyl)-1H-pyrrolo[3,2-c]pyridin-3-yl)methoxy)methyl)cyclohexanamine;
50 4-(((1H-Pyrrolo[3,2-c]pyridin-3-yl)methoxy)methyl)-1-butyl-N,N-dimethylcyclohexanamine;
51 1-Butyl-4-(((5-fluoro-2-(triethylsilyl)-1H-indol-3-yl)methoxy)methyl)-N-methylcyclohexanamine;
52 1-Butyl-N-methyl-4-(((2-(triethylsilyl)-1H-indol-3-yl)methoxy)methyl)cyclohexanamine;
53 1-Butyl-N-methyl-4-(((2-(triethylsilyl)-5-(trifluoromethyl)-1H-indol-3-yl)methoxy)methyl)cyclohexanamine;
54 1-Butyl-N-methyl-4-(((5-(trifluoromethyl)-1H-indol-3-yl)methoxy)methyl)cyclohexanamine;
55 4-(((5-Fluoro-2-(triethylsilyl)-1H-indol-3-yl)methoxy)methyl)-N,N-dimethyl-1-(thiophen-2-yl)cyclohexanamine;
56 4-(((5-Fluoro-1H-indol-3-yl)methoxy)methyl)-N,N-dimethyl-1-(thiophen-2-yl)cyclohexanamine;
57 3-(((4-(Azetidin-1-yl)-4-phenylcyclohexyl)methoxy)methyl)-5-fluoro-2-(triethylsilyl)-1H-indole;
58 3-(((4-(Azetidin-1-yl)-4-phenylcyclohexyl)methoxy)methyl)-5-fluoro-1H-indole;
60 4-(((5-Fluoro-1-methyl-2-(triethylsilyl)-1H-indol-3-yl)methoxy)methyl)-N,N-dimethyl-1-phenylcyclohexanamine;
61 4-(((5-Fluoro-1-methyl-1H-indol-3-yl)methoxy)methyl)-N,N-dimethyl-1-phenylcyclohexanamine (GRTE9052; WW447);
62 4-(((5-Fluoro-1-(methylsulfonyl)-1H-indol-3-yl)methoxy)methyl)-N,N-dimethyl-1-phenylcyclohexanamine;
63 1-(3-(((4-(Dimethylamino)-4-phenylcyclohexyl)methoxy)methyl)-5-fluoro-1H-indol-1-yl)ethanone;
64 4-(((2-tert-Butyl-5-fluoro-1H-indol-3-yl)methoxy)methyl)-N,N-dimethyl-1-phenylcyclohexanamine;
65 3-(((4-(Azetidin-1-yl)-4-phenylcyclohexyl)methoxy)methyl)-2-(triethylsilyl)-1H-pyrrolo[2,3-b]pyridine;
66 3-(((4-(Azetidin-1-yl)-4-phenylcyclohexyl)methoxy)methyl)-1H-pyrrolo[2,3-b]pyridine;
67 4-(((2-tert-Butyl-1H-pyrrolo[2,3-b]pyridin-3-yl)methoxy)methyl)-N,N-dimethyl-1-phenylcyclohexanamine;
68 N,N-Dimethyl-1-phenyl-4-(((1-(phenylsulfonyl)-1H-indol-5-yl)methoxy)methyl)cyclohexanamine;
69 4-(((1H-Indol-5-yl)methoxy)methyl)-N,N-dimethyl-1-phenylcyclohexanamine;
70 N,N-Dimethyl-1-phenyl-4-(((1-(phenylsulfonyl)-1H-indol-4-yl)methoxy)methyl)cyclohexanamine;
71 4-(((1H-Indol-4-yl)methoxy)methyl)-N,N-dimethyl-1-phenylcyclohexanamine;
72 N,N-Dimethyl-1-phenyl-4-(((2-(triethylsilyl)-5-(trifluoromethoxy)-1H-indol-3-yl)methoxy)methyl)cyclohexanamine; and
73 N,N-Dimethyl-1-phenyl-4-(((5-(trifluoromethoxy)-1H-indol-3-yl)methoxy)methyl)cyclohexanamine;
said compound optionally being in the form of a racemic mixture, or in the form of individual enantiomers or diastereomers, or in the form of mixtures of enantiomers and/or diastereomers in any mixing ratio;
or a base and/or salt of said compound or one of said forms with a physiologically compatible acid or cation.

11. Method for preparing substituted 4-aminocyclohexane derivatives according to claim 1, 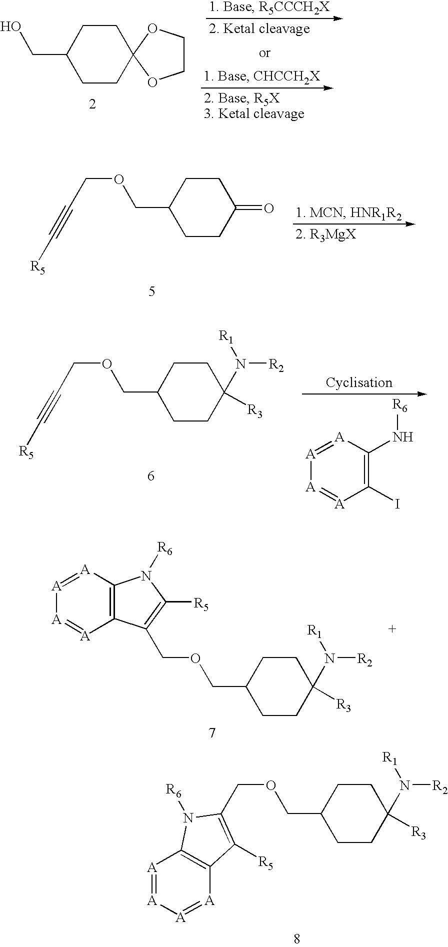
comprising:
a) reacting the ketal-protected 4-hydroxymethyl cyclohexanone 2 to yield a substituted cyclohexanone having the formula 5 in an organic solvent in the presence of an inorganic or organic base, optionally in the presence of phase-transfer catalysts, and with an alkylating agent:
R⁵CCCH₂X where X=Cl, Br
at temperatures of between −10 and 120° C. with subsequent ketal cleavage;
b) reacting the substituted cyclohexanone having the formula 5 in an aqueous medium or an organic solvent, or a mixture of these two media, in the presence of an inorganic acid, a cyanide source, and an amine:
HNR¹R²
or its hydrochloride:
HCl.HNR¹R²
at temperatures of between 20 and 60° C. and reacting the intermediate that is obtained in an organic solvent with a Grignard reagent:
MgXR³ where X=Cl, Br, I
at temperatures of between −10° C. and 40° C. to yield a monosubstituted 4-alkynyloxymethyl cyclohexylamine derivative having the formula 6;
c) reacting the monosubstituted 4-alkynyloxymethyl cyclohexylamine derivative having the formula 6 to yield the 4-aminocyclohexane derivative according to the invention having the formulae 7 and 8 in an organic solvent, in the presence of an inorganic base, in the presence of PdCl₂, Pd(OAc)₂, PdCl₂(MeCN)₂, PdCl₂(PPh₃)₂ or [1,3-bis-(2,6-diisopropylphenyl)imidazol-2-ylidene]-(3-chloropyridyl)palladium(II) chloride (PEPPSI®), optionally in the presence of additional ligands, optionally in the presence of phase-transfer catalysts, at temperatures of between 60° C. and 180° C., also with microwave assistance.

12. A pharmaceutical composition comprising a therapeutically effective amount of at least one substituted 4-aminocyclohexane derivative according to claim 1 and optionally suitable additives and/or auxiliary substances and/or optionally further active ingredients.

13. A method of treating pain in a patient in need thereof, comprising administering to said patient an effective amount therefor of a substituted 4-aminocyclohexane derivative according to claim 1.

14. The method according to claim 13, wherein the pain is selected from the group consisting of neuropathic pain, diabetic polyneuropathic pain and pain in postzosteric neuralgia.
